(19) United States
(12) Patent Application Publication
(10) Pub. No.: US 2021/0087617 A1
(43) Pub. Date: Mar. 25, 2021

(54) METHOD FOR DETECTING FLAVIVIRIDAE

(71) Applicant: NANO4 GLOBAL, LDA, Lisboa (PT)

(72) Inventors: Pedro Miguel Ribeiro Viana Baptista, Lisboa (PT); Maria Alexandra Núncio de Carvalho Ramos Fernandes, Lisboa (PT); Fabio Alexandre Teixeira Ferreira Carlos, Lisboa (PT)

(21) Appl. No.: 16/499,630

(22) PCT Filed: Feb. 22, 2019
(86) PCT No.: PCT/EP2019/054486

§ 371 Date: Sep. 30, 2019

(30) Foreign Application Priority Data
Feb. 22, 2018 (GB) 1802900.9
Feb. 7, 2019 (PT) 115295

(51) Int. Cl. C12Q 1/6837; G01N 33/52; G01N 33/569; C12Q 1/686

(52) U.S. Cl. CPC C12Q 1/6837; G01N 33/52; G01N 33/56983; C12Q 2563/155; G01N 2800/26; C12Q 2525/204; C12Q 1/686

(57) ABSTRACT

The present invention relates to health, molecular diagnostic and nanotechnology. The present invention provides a method for synthesising highly sensitive nanoprobes for use in colorimetric detection methods. The present invention provides reagents methods and kits for direct detection of viral nucleic acids in biological samples using a simple, rapid and low-cost colorimetric test.

### FIELD OF THE INVENTION

The present invention applies to the areas of health, molecular diagnosis and nanotechnology.

The present invention relates to a new colorimetric method and a kit, based on the method, for detection of specific nucleic acid sequences. These can use metal (such as gold) nanoparticles which can carry (e.g. modified) oligonucleotides, (often called probes or nanoprobes) for application in areas of biotechnology, pharmaceuticals, pharmacogenetics and medicine.

In particular, the invention relates to polynucleotides that are substantially complimentary to the part of the virus of the Flaviviridae family, for example of the genus Flavivirus, and in particular the Zika virus. It also relates to novel assay methods for detecting viral and other nucleic acids in a sample.

### BACKGROUND OF THE INVENTION

Biomolecular assays utilizing gold nanoparticles have been used for tuberculosis diagnostics (H. M. E. Azzazy and M. M. H. Mansour, Clin. Chim. Acta, 2009, 403, 1-8; P. V. Baptista, et al., Clin. Chem., 2006, 52, 1433-1434; B. Veigas, et al., in Nanodiagnostics for Tuberculosis, Understanding Tuberculosis-Global Experiences and Innovative Approaches to the Diagnosis, ed. Dr Pere-Joan Cardona, InTech, 2012, ch. 12, pp. 257-276; B. Veigas, et al., Nanotechnology, 2010, 21, 5101-5108).

Solutions containing gold nanoparticles (AuNPs) usually exhibit an intense red colour derived from the surface plasmon resonance (SPR) band centred around 520 nm; AuNP aggregation results in a red-shift of the SPR and the solution changes colour to blue (G. Doria, et al., Sensors, 2012, 12, 1657-1687). Such properties have been utilized by the present inventors previously for the detection of M. tuberculosis in an assay that relies on the colorimetric changes of a solution containing AuNPs functionalised with thiol-modified single stranded DNA oligonucleotides complementary to a target sequence from the pathogen (P. V. Baptista, et al., Clin. Chem., 2006, 52, 1433-1434).

The method relies on the hybridization between the Au-nanoprobe and the target sequence from the pathogen. In this non-cross-linking assay, the aggregation of the AuNPs is induced by salt addition and the presence of a complementary target prevents aggregation of the DNA functionalized AuNPs and the solution remains red (Veigas et al., Lab Chip, 2012, 12, 4802-4808; WO 2008/135929). The absence of a complementary target does not prevent aggregation of the DNA functionalized AuNPs, which results in a visible colour change from red to blue.

The present inventors have now identified a method for synthesizing Au-nanoprobes that are more sensitive and use of these Au-nanoprobes results in an improved method for detecting a target sequence. The present inventors have also now found surprisingly that this technology may be useful in the detection of virus RNA, particularly Zika virus RNA, in patient samples. Previous assays using colorimetric assays based on gold nanoparticles for this purpose required the use of gold nanoparticles functionalised with DNAzymes in order to achieve the necessary detetection sensitivity (J. R. Carter, et al., Virology Journal, 2013, 10, 201-215). The DNAzyme hybridizes to the target RNA and upon the addition of magnesium ions and heat digests the target RNA. Following digestion, aggregation of the DNAzyme functionalized AuNPs is induced by addition of salt and heat and a colour change is observed.

### SUMMARY OF THE INVENTION

The present invention is related to methods for producing probes suitable for use in colorimetric methods for detecting target nucleic acid sequences, particularly viral nucleic acid sequences. The probes produced according to the methods of the invention are highly sensitive to the presence of the target sequence. The present invention is related to reagents, methods and kits for direct detection of viral nucleic acids in biological samples, based on a simple, rapid and low-cost colorimetric test. The present invention is low-cost and easy to handle, which may allow the invention to be used at the point of need, in an easy and fast manner.

The present invention provides a polynucleotide comprising a sequence substantially complementary to a region within the non-structural 5 (NS5) gene of a virus of the Flaviviridae family, and compositions and/or kits comprising said polynucleotides. The present invention further provides a probe comprising a particle, optionally a gold particle (with a typical diameter of 10-20 nm) and at least one polynucleotide of the invention, and compositions and/or kits comprising said probes. The probes may comprise between 100-200 copies of the polynucleotide. The present invention also provides said probes, compositions or kits for use in a method of treatment or diagnosis or Flaviviridae virus infection, optionally Zika virus infection, or for the detection of Zikavirus.

The present invention also provides a method for preparing a probe that is suitable for use in colorimetric diagnostic assays, the method comprises contacting a particle as defined herein, with a polynucleotide, typically 10-30 nucleotides long and having a thiol group at the 3′ or 5′ end, that is capable of hybridising (i.e., binding) to a target nucleotide sequence. The target nucleotide sequence is typically a marker of a disease, for example, the target nucleotide sequence may be a viral or bacterial nucleic acid or a DNA/RNA sequence that is upregulated in a disease state (i.e., present in a patient sample at a statistically higher level than in a control sample). The method further comprises contacting the particle and the polynucleotide with a salt source, as described herein, to increase the salt concentration, wherein the salt concentration increases stepwise, as described herein.

Specifically, the present invention further provides a method for preparing a probe of the invention, the method comprising:

(a) contacting a particle with a polynucleotide of the invention; and

(b) contacting the particle and the polynucleotide with a salt source, to increase the salt concentration;

wherein the salt concentration increases stepwise.

Typically, the ratio of particle:polynucleotide (w/w) is between 150:1 and 250:1.

Typically, the salt source is a concentrated salt solution, optionally wherein the salt solution comprises a salt concentration of between 2 M and 20 M; and increasing the salt concentration stepwise comprises increasing the salt concentration by up to between 0.1-0.2 M every at least 15 minutes.

The present invention also provides a method of detecting a Flaviviridae family virus, preferably Zika virus, or nucleic acid sequence thereof in a sample, the method comprising:

(a) contacting a probe of the invention (e.g., as in any one of claims 1-7) with the sample;

(b) contacting the composition resulting from step (a) with a salt source; and

(c) detecting either:
(i) a colour change if the viral nucleic acid sequence is not present in the sample; or
(ii) no colour change if the viral nucleic acid sequence is present in the sample.

The present invention also provides an algorithm or program for a computer, for use in the methods of the invention for preparing a probe, optionally a probe of the invention, wherein the program provides the user with the volume of the concentrated salt solution required to:

(a) increase the salt concentration of the composition comprising the particle and the polynucleotide to between 0.05-0.1 M;

(b) subsequently increase the salt concentration of the composition comprising the particle and the polynucleotide by 0.1-0.2 M; and

(c) subsequently increase the salt concentration of the composition comprising the particle and the polynucleotide according to step (b) between 5-10 times, such that the final salt concentration is between 0.6-1.0 M.

The present invention also provides a device for use in detecting a target nucleic acid, such as a Flaviviridae family virus or nucleic acid sequence thereof, in a sample, the device comprising:

a. a first chamber comprising a probe specific for the target nucleic acid, which may be a Flaviviridae family virus or nucleic acid sequence therefrom; and

b. a second chamber comprising a salt source, wherein:

the device is configured to allow the probe to be contacted by the sample and, at a subsequent time, to allow the probe contacted by the sample to be contacted by the salt source.

The present invention will now be illustrated by reference to the following description, Figures and Examples, which are not intended to be limiting.

### DESCRIPTION OF THE FIGURES

FIG. 1 Schematic showing the detection method of the present invention. The method permits the recognition of a nucleic acid sequence of interest (non-amplified and/or amplified) from virus of the family Flaviviridae and/or the genus Flavivirus in biological samples via a colorimetric output mediated by metallic nanoprobes. FIG. 1A indicates the sample purification step comprising isolation of total RNA/DNA from the biological sample with or without prior lysis of cells. The collected lysate can be used immediately and/or at a later time. FIG. 1B indicates that specific RNA/DNA sequences from virus from the family Flaviviridae and the genus Flavivirus may be used directly in the method without amplification (non-amplified). FIG. 1C indicates that amplification of specific RNA/DNA sequences from virus from the family Flaviviridae and the genus Flavivirus, by PCR, RT-PCR, rRT-PCR, NASBA, LAMP, HCR may be performed. FIG. 1D indicates detection of specific RNA/DNA sequences from virus from the family Flaviviridae and the genus Flavivirus mediated by metallic nanoprobes within a concentration between 0.5 and 50 nM. Followed by addition of a solution capable of increasing the ionic strength of the media (e.g. solution of ionic strength greater than zero or a solution of ionic halide soluble in water), with a concentration comprised between 0.001 and 20 M. Both these reagents may be contained in an eye-dropper bottle for ease of use and release of dose.

FIG. 2—Illustration of the domains of the Zika virus genome. The Zika virus genome consists of single-stranded positive sense RNA molecule (10794 kb) comprising 5′ and 3′ flanking non-coding regions (5′ and 3′ NCR) and a single long open reading frame (ORF). The ORF encodes a polyprotein (5′-C-prM-E-NS1-NS2A-NS2B-NS3-NS4A-NS4B-NS5-3′) that is subsequently cleaved into capsid (C), precursor of membrane (prM), envelope (E) and seven non-structural proteins (NS).

FIG. 3—Schematic showing the inputs and outputs of the algorithm of the invention. The developed application runs an algorithm which provides the user with the volumes of solutions for use in the method for detecting Zika virus RNA of the invention. The user inputs the OD260 of the oligonucleotide (Input 1), the volume of the oligonucleotide to be used (Input 2), the ratio of particles to oligonucleotide (Input 3), the stock concentration of gold particles (Input 4), the theoretical OD of the oligonucleotide (Input 5) and the theoretical amount of the oligonucleotide in nanomoles (Input 6). The user presses the “Calculate” button. The algorithm outputs the volume of gold particle solution to add to the selected volume of oligonucleotide to achieve the selected ratio (Output 1). The second output is the volume of “Solution 1” (10 mM Tp phosphate, pH 8.0, 2% SDS) to add to the mixture of gold particles and oligonucleotide (Output 2). The algorithm also outputs the volume of “Solution 2” (10 mM Tp phosphate, pH 8.0, 1.5 M NaCl, 0.01% SDS) to add to the mixture in order to stepwise increase the final salt concentration to 0.1 M (Output 3), 0.2 M (Output 4), 0.3 M (Output 5) and 0.4 M (Output 6); with a 20 minute incubation between each addition.

FIG. 4—Colorimetric detection of Zika virus in urine samples

Graph showing detection of Zika virus RNA (NATtrol™ Zika Virus, External Run Control from ZeptoMetrix®) in Surine™ (a negative urine control from Sigma-Aldrich) as described in Example 2. A positive log value indicates no colour change in the solution, which represents non-aggregated Au-nanoprobes and hence that the sample is positive for Zika (i.e. red colour solution); a negative log value indicates a colour change in the solution, which represents aggregated Au-nanoprobes and hence that the sample is negative for Zika (i.e. blue colour solution). Samples S1-S3 contained the equivalent of 10⁹ Zika viral particles in Surine™. Samples S4-S6 contained Surine™ spiked with RNA from HCT116 human cell line control.

FIG. 5—The Microfluidic Device

The detection platform design that allows the recognition of nucleic acid sequence of interest in a biological sample via a colorimetric output mediated by metallic nanoprobes. All the channels that allow connection between chambers are highlighted in black. In Chamber B the previously purified biological sample containing nucleic acids is added (e.g. urine, saliva). In Chamber C the solution of metallic nanoprobes with a concentration comprised between 0.5 and 50 nM is inserted. In Chamber D a solution capable of increasing the ionic strength contained in Chamber C (e.g. solution of ionic strength different to zero or a solution of ionic halide soluble in water) is inserted, with a concentration comprised between 0.001-20 M. Chamber A allows the release of air trapped by the handling and operation of the microfluidic platform, avoiding reagent reflux into the original chambers. FIG. 5A describes a control detection procedure that does not contain the biological sample chamber of the system. FIG. 5B describes a detection procedure containing the biological sample.

### DETAILED DESCRIPTION OF THE INVENTION

### Introduction

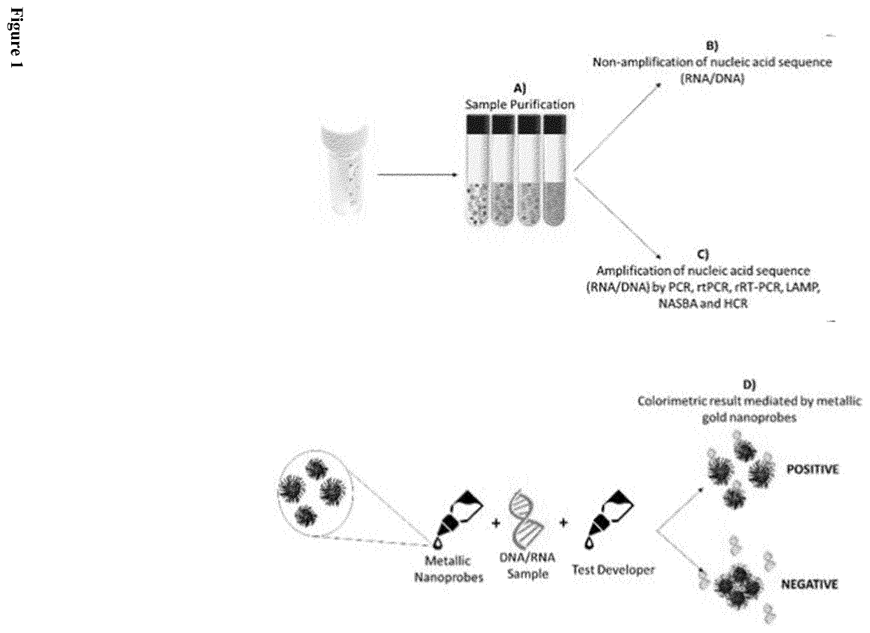

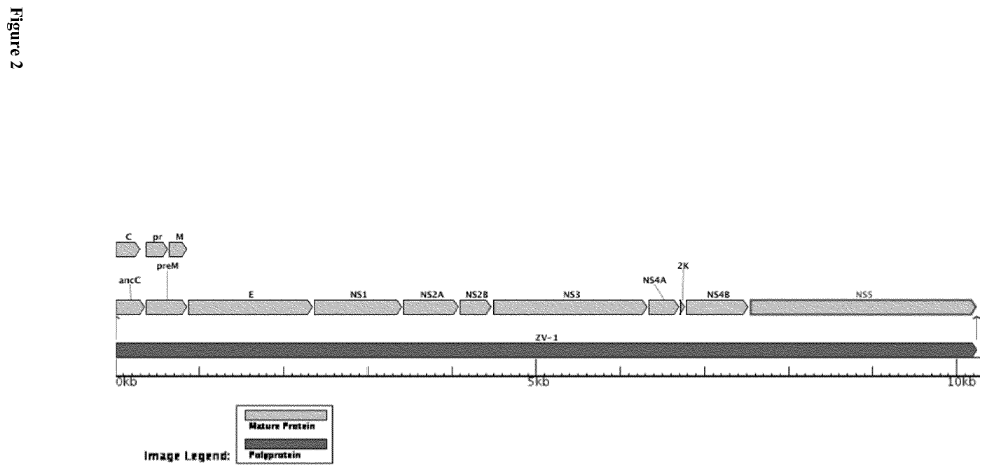

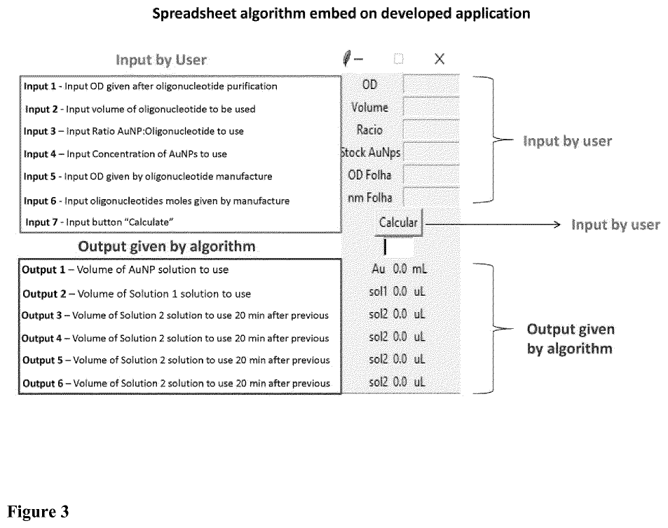

The Inventors have identified a method for producing probes, preferably gold nanoprobes, which are highly sensitive for detecting a target nucleic acid in a sample using a colorimetric method. In particular, the present inventors have identified highly sensitive and specific probes, typically comprising gold particles conjugated to a plurality of polynucleotides capable of hybridising to a target nucleic acid of interest, and methods of making such probes. The Inventors have found that a colorimetric method utilising such probes may be used for the detection of target nucleic acids of interest, preferably which target nucleic acids are associated with (i.e., diagnostic for) a particular disease or condition of interest, in biological or patient samples. In particular the probes may be used for the detection of nucleic acids from virus of the Flaviviridae family and the genus Flavivirus, in biological or patient samples. In particular, the Inventors have found that patient samples may be mixed with gold probes which are functionalised with polynucleotides, usually single stranded DNA or RNA sequences, which are complementary to a region within the non-structural 5 (NS5) gene of virus of the Flaviviridae, to detect the presence of said virus in the patient sample (FIG. 1). In particular, the Inventors have identified the regions of the genomes of virus of the Flaviviridae family and the genus Flavivirus that are most useful for detection by such methods. An exemplary virus of this family and genus is Zika virus. The methods of the present invention may be applied to Zika virus. The Zika virus genome consists of a single-stranded positive sense RNA molecule (SEQ ID NO 1) comprising a single open reading frame (ORF) encoding a polyprotein that is cleaved into capsid (C), precursor of membrane (prM), envelope (E) and seven non-structural proteins (NS) (FIG. 2) (T. J. Chambers, et al., (1990) Annu Rev Microbiol 44: 649-688; G. Kuno and G-J J Chang (2007) Arch Virol 152: 687-696) (FIG. 2). The NS5 protein (˜103 kDa) is the largest viral protein (SEQ ID NO 5). The C-terminal portion of the NS5 protein has RNA-dependent RNA polymerase (RdRP) activity and the N-terminal portion is involved in RNA capping (Lindenbach B D and Rice C M (2003) Adv Virus Res 59: 23-61). The envelope and other non-structural genes (NS5) have been used previously for phylogenetic analysis due to their high phylogenetic signal contents. Broadly, these studies have outlined that all Zika virus strains/isolates cluster into two major clades, one representing the African and the other the Asian lineage (Faye O, et al., (2014) PLoS Negl Trop Dis. 8:1-10). The present Inventors have identified that the NS3 gene (SEQ ID NO 4), the NS5 gene (SEQ ID NO 5) and the envelope gene (SEQ ID NO 2) are most useful for detection using a colorimetric assay. In particular, the NS5 gene (SEQ ID NO 5) is useful for detection of Zika virus or other virus of the Flaviviridae family in biological or patient samples.

### Polynucleotides

Polynucleotides having any sequence are useful in the probes of the present invention. Typically such polynucleotides comprise or consist of DNA, RNA, or DNA and RNA, preferably DNA. The polynucleotides typically are capable of hybridising to (i.e., binding to) and are optionally complementary to, a target nucleic acid of interest (i.e., a target nucleic acid sequence). Suitable target nucleic acids are described further herein, but include any nucleic acid that is associated with a disease or condition, for example the presence of a target nucleic acid in a sample from a patient may be indicative that said patient has a particular disease or condition. In one particular aspect, the present invention provides a polynucleotide comprising a sequence substantially complementary to a region within the non-structural 5 (NS5) gene of virus of the Flaviviridae family.

The terms polynucleotide and oligonucleotide may be used interchangeably. In the context of this invention, the term “polynucleotide” refers to a polymer of nucleotide or nucleoside monomers consisting of naturally-occurring bases, sugars and intersugar (backbone) linkages. The term “polynucleotide” also includes polymers comprising non-naturally occurring monomers, or portions thereof, which function similarly. Polynucleotides are generally classified as deoxyribooligonucleotides or ribooligonucleotides, which are oligomers of DNA or RNA molecules. A deoxyribooligonucleotide consists of a 5-carbon sugar (deoxyribose) which is joined covalently to phosphate at the 5′ and 3′ carbons of the sugar to form an alternating, unbranched polymer. A ribooligonucleotide consists of a similar repeating structure where the 5-carbon sugar is ribose.

The term “complementary” may be considered to describe two polynucleotides comprising sequences such that when they are aligned antiparallel to each other, the nucleotide bases at each position in the sequences will be complementary and capable of hybridising, i.e. complementary Watson-Crick base pairing can occur. Such complementary base pairing includes A-T/U base pairing and G-C base pairing. The term “substantially complementary” may be considered to describe two polynucleotides comprising sequences with perfect complementary Watson-Crick base pairing. The term “substantially complementary” may also be considered to describe two polynucleotides comprising sequences wherein the complementary Watson-Crick base pairing is incomplete. For example, when the substantially complementary sequences of the two polynucleotides are aligned antiparallel to each other, there are some nucleotides of the sequences that are not base-paired (i.e. not capable of base-pairing) with the corresponding nucleotide on the substantially complementary strand. For example, up to 1%, 2%, 3%, 4%, 5%, 10%, 15%, 20%, 25%, 30% or 40% of the nucleotides of the complementary sequences of the two polynucleotides may be mismatched (i.e. not capable of base-pairing, not complementary).

Two (i.e. a pair of) polynucleotides that are substantially complementary can in some embodiments be characterised as capable of binding or hybridising to each other, for example, through nucleotide base pairing. Polynucleotides will be capable of binding to each other if at least 60%, 65%, 70%, 75%, 80%, 85%, 90%, 95%, 98% or 100%, preferably at least 80%, more preferably between 90-100% of the nucleotides within the complementary sequence in the first polynucleotide are matched with the corresponding complementary sequence on the second polynucleotide. This may be determined by sequencing the polynucleotides and comparing the sequences. Methods for testing binding or hybridisation of polynucleotides are well known in the art.

Sequence identity, including determination of sequence complementarity for nucleic acid or polynucleotide sequences, may be determined by sequence comparison and alignment algorithms known in the field. To determine the percent identity of two nucleic acid sequences (or polynucleotide sequences), the sequences are aligned for optimal comparison purposes (e.g., gaps can be introduced in the first sequence or second sequence for optimal alignment). The nucleotides at corresponding nucleotide positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions*100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilised for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimised by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimised by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package.

The polynucleotides described herein may comprise single-stranded DNA or single-stranded RNA. The length of the polynucleotide may be between 10 and 100 nucleotides; between 10 and 50 nucleotides; between 10 and 30 nucleotides; or between 10 and 25 nucleotides; optionally at least 10 nucleotides; at least 14 nucleotides, at least 16 nucleotides; at least 20 nucleotides; at least 22 nucleotides; at least 24 nucleotides; or at least 30 nucleotides in length. Most usually the polynucleotide is between 10 and 30 nucleotides in length.

The polynucleotide may further comprise a thiol (SH) functional group. Preferably, the thiol group may be at the 3′ or 5′ terminus of the polynucleotide. The thiol may optionally be at any position on the polynucleotide. Other functional groups may also be used, for example amine; phosphine; and dithiol groups. Methods for functionalising polynucleotides with thiols are well known in the art.

Polynucleotides functionalised with thiols, preferably at their 3′- or 5′-ends, readily attach to gold particles. In the methods of the invention the thiol-modified polynucleotide sequences attach to particles (nanoparticles), for example gold particles (gold nanoparticles). The thiol group forms a quasi-covalent bond with the metallic particles. The polynucleotide may further comprise a linker between the thiol group and the polynucleotide. The linker may comprise a short oligonucleotide, optionally the linker may comprise 5-100 nucleotides, 5-50 nucleotides, 5-30 nucleotides, 5-25 nucleotides, 5-10 nucleotides or 5, 6, 7, 8, 9, 10, 15, 20, 25, 30, 40, 50 or 100 nucleotides. Preferably, the linker comprises 5-10 nucleotides. The oligonucleotide linker may be AT-rich. The oligonucleotide linker may comprise at least 10%, at least 20%, at least 30%, at least 40% , at least 50%, at least 60%, at least 70%, at least 80%, at least 90% or 100% A and T nucleotides; preferably at least 80% A and T nucleotides. In some embodiments, the linker may comprise n alkyl groups, wherein n may be 5-100, 5-50, 5-30, 5-25, 5-10 or 5, 6, 7, 8, 9, 10, 15, 20, 25, 30, 40, 50 or 100; preferably n is 5-10.

In one embodiment, the polynucleotides of the present invention comprise a sequence that is substantially complementary to a region within the non-structural 5 (NS5) gene of virus of the Flaviviridae family. The terms “complementary to a region”, “target a region” or “bind to a region” or “hybridise to a region” are used interchangeably and have the same meanings for the purposes of this application. The polynucleotides of the invention are capable of hybridising to sequences found within the genome of viruses of the Flaviviridae family. Being “capable of hybridising to” is equivalent to being “substantially complementary to”.

In one embodiment, the virus may be of the Flaviviridae family and the genus Flavivirus. Within this genus, the virus may be selected from the group consisting of Zika virus, West Nile virus, dengue virus, tick-borne encephalitis virus, yellow fever virus, Japanese encephalitis virus, cell fusing agent virus (CFAV), Palm Creek virus (PCV), and Parramatta River virus (PaRV). The virus may be Dengue virus or Zika virus. Preferably the virus is Zika virus. Preferably, the polynucleotide comprises a sequence substantially complementary to a region within the non-structural 5 (NS5) gene of Zika virus (SEQ ID NO 5).

In one embodiment the polynucleotide may comprise a sequence substantially complementary to a region within the gene encoding the RNA-dependent RNA polymerase protein or the 3′-untranslated region of the viral genome. In some embodiments the polynucleotide comprises a sequence substantially complementary to a region within the non-structural 5 (NS5) gene wherein the sequence of the NS5 gene has at least 60, 65, 70, 75, 80, 85, 90, 91, 92, 93, 94, 95, 96, 97, 98, 99 or 100% sequence identity to SEQ ID NO. 5 (sequence of the Zika virus NS5 gene); preferably at least 80% sequence identity to SEQ ID NO. 5. Preferably, the polynucleotide comprises a sequence substantially complementary to a region within the non-structural 5 (NS5) gene of Zika virus (SEQ ID NO 5).

In some embodiments, the polynucleotide comprises a sequence substantially complementary to a region within the viral genome that has a sequence with at least 60%, 70%, 75%, 80%, 85%, 90%, 95%, 96%, 97%, 98%, 99% or 100% sequence identity; preferably at least 80% sequence identity, to the sequence between nucleotides 9182 to 9961 of SEQ ID NO 1 (Zika virus genome sequence). Preferably, the polynucleotide of the invention comprises a sequence substantially complementary to a region within the viral genome that has the sequence between nucleotides 9182 to 9961 of SEQ ID NO 1 (Zika virus genome sequence). Thus, in some embodiments the polynucleotides of the invention will be capable of hybridising to a region of the viral genome having the sequence between nucleotides 9182 to 9961 of SEQ ID NO 1.

In some embodiments, the polynucleotides of the invention comprise a sequence substantially complementary to a region within the viral genome that has a sequence having at least 60%, 70%, 75%, 80%, 85%, 90%, 95%, 96%, 97%, 98%, 99% or 100% sequence identity to; preferably at least 80% sequence identity to the sequence selected from SEQ ID NO 13, SEQ ID NO 14, SEQ ID NO 15, SEQ ID NO 16 or SEQ ID NO 17. In some embodiments, the polynucleotide comprises a sequence substantially complementary to a region within the viral genome that has a sequence having at least 80% sequence identity to SEQ ID NO 13. In some embodiments, the polynucleotide comprises a sequence substantially complementary to a region within the viral genome that has a sequence having at least 80% sequence identity to SEQ ID NO 14. In some embodiments, the polynucleotide comprises a sequence substantially complementary to a region within the viral genome that has a sequence having at least 80% sequence identity to SEQ ID NO 15. In some embodiments, the polynucleotide comprises a sequence substantially complementary to a region within the viral genome that has a sequence having at least 80% sequence identity to SEQ ID NO 16. In some embodiments, the polynucleotide comprises a sequence substantially complementary to a region within the viral genome that has a sequence having at least 80% sequence identity to SEQ ID NO 17.

In some embodiments, the polynucleotides of the invention comprise a sequence substantially complementary to a region within the viral genome that has the sequence selected from SEQ ID NO 13, SEQ ID NO 14, SEQ ID NO 15, SEQ ID NO 16 or SEQ ID NO 17. In some embodiments the polynucleotide comprises a sequence substantially complementary to a region within the viral genome that has the sequence of SEQ ID NO 13. In some embodiments the polynucleotide comprises a sequence substantially complementary to a region within the viral genome that has the sequence of SEQ ID NO 14. In some embodiments the polynucleotide comprises a sequence substantially complementary to a region within the viral genome that has the sequence of SEQ ID NO 15. In some embodiments the polynucleotide comprises a sequence substantially complementary to a region within the viral genome that has the sequence of SEQ ID NO 16. In some embodiments the polynucleotide comprises a sequence substantially complementary to a region within the viral genome that has the sequence of SEQ ID NO 17.

In some embodiments, the polynucleotides of the invention comprise a sequence with at least 60%, 70%, 75%, 80%, 85%, 90%, 95%, 96%, 97%, 98%, 99% or 100% sequence identity to; preferably at least 80% sequence identity to, one of SEQ ID NO 6 (ProbeR1Zika); SEQ ID NO 7 (ProbeR2Zika); SEQ ID NO 8 (ProbeR3Zika); SEQ ID NO 9 (ProbeZikaUniversalPolyA); SEQ ID NO 10 (ProbeZikaUniversal); SEQ ID NO 11 (ProbeZikaDegenel) or SEQ ID NO 12 (ProbeZikaDegene2). In some embodiments the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 6. In some embodiments the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 7. In some embodiments the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 8. In some embodiments the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 9. In some embodiments the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 10. In some embodiments the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 11. In some embodiments the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 12.

In some preferred embodiments, the polynucleotides of the invention comprise a sequence of one of SEQ ID NO 6; SEQ ID NO 7; SEQ ID NO 8; SEQ ID NO 9; SEQ ID NO 10; SEQ ID NO 11 or SEQ ID NO 12. In one preferred embodiment, the polynucleotide comprises a sequence of SEQ ID NO 6. In one preferred embodiment, the polynucleotide comprises a sequence of SEQ ID NO 7. In one preferred embodiment, the polynucleotide comprises a sequence of SEQ ID NO 8. In one preferred embodiment, the polynucleotide comprises a sequence of SEQ ID NO 9. In one preferred embodiment, the polynucleotide comprises a sequence of SEQ ID NO 10. In one preferred embodiment, the polynucleotide comprises a sequence of SEQ ID NO 11. In one preferred embodiment, the polynucleotide comprises a sequence of SEQ ID NO 12.

It is apparent to one of skill in the art that the sequences SEQ ID NO 6; SEQ ID NO 7; SEQ ID NO 8; SEQ ID NO 9; SEQ ID NO 10; SEQ ID NO 11 or SEQ ID NO 12 are complementary respectively to sequences SEQ ID NO 13, SEQ ID NO 14, SEQ ID NO 15, SEQ ID NO 16 or SEQ ID NO 17. Thus polypeptides having a sequence with at least 80% sequence identity to or a sequence of SEQ ID NO 6; SEQ ID NO 7; SEQ ID NO 8; SEQ ID NO 9; SEQ ID NO 10; SEQ ID NO 11 or SEQ ID NO 12 will be a polynucleotide comprising a sequence substantially complementary to a region within the viral genome, wherein the virus is Zika virus. Such polynucleotides are capable of hybridising to the Zika virus genome and thus are useful in the kits or methods of the invention for detecting Zika virus or nucleic acid thereof.

In some embodiments any of the polynucleotides described herein may comprise a sequence that is hybridised (bound) to (a portion of) a virus nucleic acid sequence. Optionally, the virus nucleic acid sequence comprises single or double stranded DNA or RNA, preferably RNA, and more preferable single-stranded RNA. The virus nucleic acid sequence may consist of the virus genome, but typically the virus nucleic acid sequence comprises a portion of the virus genome. This portion may be a fragment of the virus genome, for example, the virus nucleic acid may comprise at least 10, 20, 30, 40, 50, 100, 200, 300, 400, 500, 1000, 2000, 3000, 4000, 5000, 8000 nucleotides of the virus genome, preferably between 10-1000 nucleotides of the virus genome. In some embodiments, the virus nucleic acid is derived from, has been ejected from or removed from a virus of the Flaviviridae family, preferably of the genus Flavivirus. In some preferred embodiments the virus is Zika virus.

The polynucleotides of the invention are for use in a method of treatment or diagnosis. In some embodiments the polynucleotides of the invention are for use in a method of diagnosing viral infection in a patient, preferably by detecting the presence (or absence if the patient is not infected) of the virus or virus nucleic acid. In a preferred embodiment, the polynucleotides of the invention are for use in a method of diagnosing Zika virus infection in a patient. In a second preferred embodiment, the polynucleotides of the invention are for use in a method of detecting Zika virus or Zika virus nucleic acid (single-stranded RNA) in a patient sample. In one embodiment, the use of the polynucleotides of the invention in the manufacture of a diagnostic for diagnosing Zika virus infection or detecting Zika virus nucleic acid in a sample is contemplated.

Any of the probes of the invention, as described below, may comprise any of the nucleotides of the invention described herein. Any of the nucleotides of the invention, as described above, are useful in the methods of detecting a Flaviviridae family virus or nucleic acid sequence thereof described herein.

### Probes

The present invention also provides a probe comprising a particle and a polynucleotide, preferably the present invention provides a probe comprising a particle and a plurality of polynucleotides. In some aspects, the present invention also provides a probe comprising a particle and a polynucleotide of the invention, relating to Zika virus as defined herein above. The terms “particle” and “nanoparticle” are used herein interchangeably. Both are terms of art that would be understood by the skilled person. Typically the term “particle” or “nanoparticle” describes a very small, nanoscale object with an inorganic core, typically such “particles” or “nanoparticles” are between 1 and 100 nm in size. In some embodiments the probes of the invention comprise a particle made of metal.

Preferably, the particle may be made of a metal selected from the group consisting of gold, silver, a gold/silver alloy or an alloy of gold with another metal or the particles may comprise a combination of one or more of these. In a preferred embodiment, the particles are made of gold. Methods for preparing particles suitable for use in the nanoprobes of the invention, for example gold particles, are well known in the art, and include, for example, the gold salt reduction method with a citrate salt (using for example HAuCl4) (see, for example, Turkevich, J.; Stevenson, P. C.; Hillier, J. Discuss. Faraday Soc. 1951, 11, 55).

The particles may be any shape, for example, substantially spherical, cylindrical, triangular, cubic, prismatic or any other geometrical shape. The particles may be hollow or solid. In a preferred embodiment the particles are substantially spherical. The diameter of the particles may be between 1 and 100 nm; 5 and 80 nm; 10 and 50 nm; 10 and 40 nm; 10 and 30 nm; 10 and 20 nm; 12 and 48 nm or 14 and 17. In a preferred embodiment the particles are substantially spherical with a diameter of 14 to 17 nm.

The terms “probe” and “nanoprobe” are used herein interchangeably. The terms “probe” and “nanoprobe” can be used to refer to a “particle” or “nanoparticle” as defined herein conjugated or linked to a polynucleotide, preferably a plurality of polynucleotides, optionally wherein the polynucleotide is a polynucleotide of the invention as described above. The probes may comprise any of the polynucleotide sequences of the invention as defined herein. Probes typically consist of a particle (preferably a substantially spherical, gold particle with a diameter of 14-17 nm), which is conjugated or linked to a polynucleotide, preferably a plurality of polynucleotides, optionally wherein the polynucleotide comprises a polynucleotide sequence of the invention, through a thiol group (preferably wherein the thiol group is at the 3′ or 5′ terminus of the polynucleotide) on the polynucleotide. Optionally, the thiol group and the polynucleotide sequence are separated by a linker as defined above. Typically a thiol group attached to the 3′ or 5′ terminus of the polynucleotide, preferably the plurality of polynucleotides, optionally wherein the polynucleotide comprises polynucleotide of the invention, forms a quasi-covalent bond with metallic particles. In this manner the particles may be functionalised with the polynucleotide, preferably the plurality of polynucleotides, optionally the polynucleotides of the invention, to form the probes (or nanoprobes) of the invention. The terms “conjugated to”, “linked to” and “functionalised with” are used interchangeably and have the same meaning for the purposes of this application.

In one aspect the particles described herein are functionalised with (conjugated to) a polynucleotide, preferably a plurality of polynucleotides. The polynucleotide may have any sequence. The polynucleotide may comprise or consist of DNA, RNA or DNA and RNA, preferably the polynucleotide comprises or consists of DNA. The polynucleotide is typically single-stranded and between 5-100, 10-75, 10-50, or preferably between 10-30 nucleotides in length. The polynucleotide may further comprise a linker at the 3′ and/or 5′ end of the polynucleotide. The polynucleotide may comprise a linker and the linker may comprise nucleotides, optionally between 1-10, preferably between 1-5 nucleotides. Typically, the length of the polynucleotide defined herein does not include the nucleotides of the linker. The length of the polynucleotide defined herein typically describes the number of nucleotides that hybridise to the target nucleic acid sequence. The polynucleotide (optionally including the linker) preferably comprises a thiol group at the 3′ and/or 5′ end. The thiol group allows for conjugation to the metal, preferably gold, particle. In a preferred embodiment substantially spherical gold nanoparticles, optionally with a diameter between 14-17 nm, are functionalised with (conjugated to) a polynucleotide, preferably a plurality of polynucleotides. In some aspects the polynucleotide comprises or consists of a polynucleotide of the invention as described herein.

The polynucleotide typically hybridises to (i.e., binds to, and optionally is complementary to) a sequence within a target nucleic acid. The target nucleic acid may be any nucleic acid of interest. The target nucleic acid sequence may comprise DNA or RNA, preferably RNA. The target nucleic acid may be any length, but will comprise a sequence that hybridises to (binds to/is complementary to) the polynucleotide. The target nucleic acid may be associated with a disease phenotype. The target nucleotide sequence may be a marker of a disease, for example, the target nucleotide sequence may be upregulated in a disease state. For example, the target nucleic acid may be present in a patient sample at a statistically higher level than in a control sample, wherein the control sample may be a healthy subject or control value averaged from a number of healthy subjects. Typically the target nucleic acid is associated with a disease or condition and in preferred aspects detection of the target nucleic acid may allow for diagnosis of the disease or condition. Thus, in some aspects detection of the target nucleic acid, for example in a sample from a patient, is indicative of a disease or condition. The disease or condition may be any disease which is associated with a particular nucleic acid, including for example, bacterial infection, viral infection, cancer or organ or tissue transplant rejection, preferably bacterial or viral infection. The target nucleic acid may be isolated from a patient or subject suspected of having or having a disease. The target nucleic acid may be a bacterial nucleic acid, a viral nucleic acid or a disease biomarker, e.g., a cancer biomarker, preferably a bacterial nucleic acid or a viral nucleic acid, most preferably a viral nucleic acid. In some preferred embodiments the target nucleic acid is a Flavividae virus RNA, preferably a Zika virus RNA, for example as described herein.

In one preferred embodiment substantially spherical gold nanoparticles with a diameter between 14-17 nm are functionalised with (conjugated to) a polynucleotide comprising a sequence with at least 60%, 70%, 75%, 80%, 85%, 90%, 95%, 96%, 97%, 98%, 99% or 100% sequence identity to; preferably at least 80% sequence identity to, one of SEQ ID NO 6; SEQ ID NO 7; SEQ ID NO 8; SEQ ID NO 9; SEQ ID NO 10; SEQ ID NO 11 or SEQ ID NO 12; through a thiol group at the 3′ or 5′ terminus of the polynucleotide; optionally wherein the polynucleotide and the thiol group are connected by a linker. Preferably wherein said linker comprises 5-10 nucleotides. In one preferred embodiment the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 6. In one preferred embodiment the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 7. In one preferred embodiment the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 8. In one preferred embodiment the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 9. In one preferred embodiment the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 10. In one preferred embodiment the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 11. In one preferred embodiment the polynucleotide comprises a sequence with at least 60%, 70%, 75%, 80%, 85%, 90%, 95%, 96%, 97%, 98%, 99% or 100% sequence identity to; preferably at least 80% sequence identity to SEQ ID NO 12.

In a particularly preferred embodiment substantially spherical gold nanoparticles with a diameter between 14-17 nm are functionalised with (conjugated to) a polynucleotide comprising a sequence of one of SEQ ID NO 6; SEQ ID NO 7; SEQ ID NO 8; SEQ ID NO 9; SEQ ID NO 10; SEQ ID NO 11 or SEQ ID NO 12; through a thiol group at the 3′ or 5′ terminus of the polynucleotide; optionally wherein the polynucleotide and the thiol group are connected by a linker. Preferably wherein said linker comprises 5-10 nucleotides. In one preferred embodiment the polynucleotide comprises a sequence of SEQ ID NO 6. In one preferred embodiment the polynucleotide comprises a sequence of SEQ ID NO 7. In one preferred embodiment the polynucleotide comprises a sequence of SEQ ID NO 8. In one preferred embodiment the polynucleotide comprises a sequence of SEQ ID NO 9. In one preferred embodiment the polynucleotide comprises a sequence of SEQ ID NO 10. In one preferred embodiment the polynucleotide comprises a sequence of SEQ ID NO 11. In one preferred embodiment the polynucleotide comprises a sequence of SEQ ID NO 12.

The probes of the invention typically comprise one particle and at least one polynucleotide, preferably a plurality of polynucleotides. Preferably, the plurality of polynucleotides conjugated to the particle all have the same sequence (i.e., the particle comprises multiple copies of a polynucleotide). Alternatively, the particle may comprise a plurality of polynucleotides having different sequences. The particle may comprise a plurality of polynucleotides, wherein the plurality comprises 1, 2, 3, 4, 5, 6, 7, 8, 9, 10 or more, preferably 1-3 different polynucleotide sequences. The probes of the invention typically comprise one particle and between 10-400, 20-350, 50-300, 100-200 or 140-180 polynucleotides, preferably 140-180 polynucleotides. In preferred aspects each of these polynucleotides have the same sequences. Thus, the probes of the invention may comprise one particle conjugated to 10-400, 20-350, 50-300, 100-200 or 140-180 polynucleotides, preferably 140-180 polynucleotides. The probes of the invention typically comprise a particle wherein the density of polynucleotides conjugated to the particle is between 1-100 pmol/cm², 10-80 pmol/cm², 15-50 pmol/cm², 20-40 pmol/cm² or 20-30 pmol/cm²; preferably 20-30 pmol/cm². In one embodiment, each probe comprises one particle and multiple copies (a plurality) of one of the polynucleotide sequences of the invention as described above. At least 1 and typically between 10-400, 20-350, 50-300, 100-200 or 140-180 copies; preferably 140-180 copies of a polynucleotide of the invention may be attached to one particle of the invention to form one nanoprobe of the invention. The density of polynucleotides conjugated to the particle in the probes of the invention is typically between 1-100 pmol/cm², 10-80 pmol/cm², 15-50 pmol/cm², 20-40 pmol/cm² or 20-30 pmol/cm²; preferably 20-30 pmol/cm². The density of polynucleotides and/or the number of polynucleotides conjugated to the particles as described herein has surprisingly been found to provide probes that are highly sensitive and highly specific in colorimetric assays for detecting the nucleic acid of interest, as described herein.

The probes of the present invention for use in a method of treatment or diagnosis is also contemplated. For example, the present invention provides a probe as described herein for use in a method of diagnosis or for use in a method of treatment. In one aspect, the present invention provides a probe as described herein for use in a method of diagnosing a disease or condition selected from bacterial infection, viral infection or cancer, preferably bacterial or viral infection. The probes may be used in a method of treatment or diagnosis, such as detecting Zika virus (typically by detecting a Zika virus nucleic acid) or diagnosing Zika virus infection. The probes may be used in the manufacture of a medicament or diagnostic for diagnosing Zika virus infection or detecting Zika virus (typically by detecting a Zika virus nucleic acid). Any of the probes of the invention, as described herein, are useful in methods of detecting a target nucleic acid of interest, preferably which target nucleic acid is associated with a disease or condition. Particularly, any of the probes of the invention conjugated to a polynucleotide of the invention as described herein, are useful in the methods of detecting a Flaviviridae family virus or nucleic acid sequence thereof also described herein.

### Compositions and Kits

The invention also provides compositions and/or kits comprising the polynucleotide sequences or probes of the invention.

### Compositions or Kits Comprising the Nucleotides of the Invention

The compositions or kits of the present invention may comprise any polynucleotides according to the invention or a plurality of polynucleotides according to the invention. The compositions may optionally further comprise buffered solutions, for example phosphate buffers, HEPES buffers, Tris buffers, optionally wherein the pH is between 6-9, preferably between 7-8. In one embodiment the composition/kit may comprise a polynucleotide comprising a sequence with at least 60, 65, 70, 75, 80, 85, 90, 91, 92, 93, 94, 95, 96, 97, 98, 99 or 100% sequence identity, preferably 80-100%, more preferably at least 80% sequence identity to a sequence selected from the group consisting of SEQ ID NO 6 (ProbeRiZika); SEQ ID NO 7 (ProbeR2Zika); SEQ ID NO 8 (ProbeR3Zika); SEQ ID NO 9 (ProbeZikaUniversalPolyA); SEQ ID NO 10 (ProbeZikaUniversal); SEQ ID NO 11 (ProbeZikaDegenel) and SEQ ID NO 12 (ProbeZikaDegene2). The composition/kit may comprise at least one or a plurality of these polynucleotide sequences in any combination. In one embodiment the composition/kit comprises a polynucleotide comprising a sequence with at least 80% sequence identity to SEQ ID NO 6. In one embodiment the composition/kit comprises a polynucleotide comprising a sequence with at least 80% sequence identity to SEQ ID NO 7. In one embodiment the composition/kit comprises a polynucleotide comprising a sequence with at least 80% sequence identity to SEQ ID NO 8. In one embodiment the composition/kit comprises a polynucleotide comprising a sequence with at least 80% sequence identity to SEQ ID NO 9. In one embodiment the composition/kit comprises a polynucleotide comprising a sequence with at least 80% sequence identity to SEQ ID NO 10. In one embodiment the composition/kit comprises a polynucleotide comprising a sequence with at least 80% sequence identity to SEQ ID NO 11. In one embodiment the composition/kit comprises a polynucleotide comprising a sequence with at least 80% sequence identity to SEQ ID NO 12. In one embodiment the composition/kit comprises a polynucleotide comprising a sequence with at least 80% sequence identity to SEQ ID NO 6 and a polynucleotide comprising a sequence with at least 80% sequence identity to SEQ ID NO 7. In one embodiment the composition/kit comprises a polynucleotide comprising a sequence with at least 80% sequence identity to SEQ ID NO 6, a polynucleotide comprising a sequence with at least 80% sequence identity to SEQ ID NO 7 and a polynucleotide comprising a sequence with at least 80% sequence identity to SEQ ID NO 8. In one embodiment the composition/kit comprises a polynucleotide comprising a sequence with at least 80% sequence identity to SEQ ID NO 6, a polynucleotide comprising a sequence with at least 80% sequence identity to SEQ ID NO 7, a polynucleotide comprising a sequence with at least 80% sequence identity to SEQ ID NO 8 and a polynucleotide comprising a sequence with at least 80% sequence identity to SEQ ID NO 9. In one embodiment the composition/kit comprises a polynucleotide comprising a sequence with at least 80% sequence identity to SEQ ID NO 6, a polynucleotide comprising a sequence with at least 80% sequence identity to SEQ ID NO 7, a polynucleotide comprising a sequence with at least 80% sequence identity to SEQ ID NO 8, a polynucleotide comprising a sequence with at least 80% sequence identity to SEQ ID NO 9 and a polynucleotide comprising a sequence with at least 80% sequence identity to SEQ ID NO 10. In one embodiment the composition/kit comprises a polynucleotide comprising a sequence with at least 80% sequence identity to SEQ ID NO 6, a polynucleotide comprising a sequence with at least 80% sequence identity to SEQ ID NO 7, a polynucleotide comprising a sequence with at least 80% sequence identity to SEQ ID NO 8, a polynucleotide comprising a sequence with at least 80% sequence identity to SEQ ID NO 9, a polynucleotide comprising a sequence with at least 80% sequence identity to SEQ ID NO 10 and a polynucleotide comprising a sequence with at least 80% sequence identity to SEQ ID NO 11.

In a preferred embodiment the composition/kit comprises a polynucleotide comprising a sequence with at least 80% sequence identity to SEQ ID NO 6, a polynucleotide comprising a sequence with at least 80% sequence identity to SEQ ID NO 7, a polynucleotide comprising a sequence with at least 80% sequence identity to SEQ ID NO 8, a polynucleotide comprising a sequence with at least 80% sequence identity to SEQ ID NO 9, a polynucleotide comprising a sequence with at least 80% sequence identity to SEQ ID NO 10 and a polynucleotide comprising a sequence with at least 80% sequence identity to SEQ ID NO 11 and a polynucleotide comprising a sequence with at least 80% sequence identity to SEQ ID NO 12.

In one embodiment the composition/kit may comprise a polynucleotide comprising a sequence selected from the group consisting of SEQ ID NO 6 (ProbeR1Zika); SEQ ID NO 7 (ProbeR2Zika); SEQ ID NO 8 (ProbeR3Zika); SEQ ID NO 9 (ProbeZikaUniversalPolyA); SEQ ID NO 10 (ProbeZikaUniversal); SEQ ID NO 11 (ProbeZikaDegenel) and SEQ ID NO 12 (ProbeZikaDegene2). The composition/kit may comprise a plurality of these polynucleotides in any combination. In one embodiment the composition/kit comprises a polynucleotide comprising the sequence of SEQ ID NO 6. In one embodiment the composition/kit comprises a polynucleotide comprising the sequence of SEQ ID NO 7. In one embodiment the composition/kit comprises a polynucleotide comprising the sequence of SEQ ID NO 8. In one embodiment the composition/kit comprises a polynucleotide comprising the sequence of SEQ ID NO 9. In one embodiment the composition/kit comprises a polynucleotide comprising the sequence of SEQ ID NO 10. In one embodiment the composition/kit comprises a polynucleotide comprising the sequence of SEQ ID NO 11. In one embodiment the composition/kit comprises a polynucleotide comprising the sequence of SEQ ID NO 12. In one embodiment the composition/kit comprises a polynucleotide comprising the sequence of SEQ ID NO 6 and a polynucleotide comprising the sequence of SEQ ID NO 7. In one embodiment the composition/kit comprises a polynucleotide comprising the sequence of SEQ ID NO 6, a polynucleotide comprising the sequence of SEQ ID NO 7 and a polynucleotide comprising the sequence of SEQ ID NO 8. In one embodiment the composition/kit comprises a polynucleotide comprising the sequence of SEQ ID NO 6, a polynucleotide comprising the sequence of SEQ ID NO 7, a polynucleotide comprising the sequence of SEQ ID NO 8 and a polynucleotide comprising the sequence of SEQ ID NO 9. In one embodiment the composition/kit comprises a polynucleotide comprising the sequence of SEQ ID NO 6, a polynucleotide comprising the sequence of SEQ ID NO 7, a polynucleotide comprising the sequence of SEQ ID NO 8, a polynucleotide comprising the sequence of SEQ ID NO 9 and a polynucleotide comprising the sequence of SEQ ID NO 10. In one embodiment the composition/kit comprises a polynucleotide comprising the sequence of SEQ ID NO 6, a polynucleotide comprising the sequence of SEQ ID NO 7, a polynucleotide comprising the sequence of SEQ ID NO 8, a polynucleotide comprising the sequence of SEQ ID NO 9, a polynucleotide comprising the sequence of SEQ ID NO 10 and a polynucleotide comprising the sequence of SEQ ID NO 11.

In a preferred embodiment the composition/kit comprises a polynucleotide comprising the sequence of SEQ ID NO 6, a polynucleotide comprising the sequence of SEQ ID NO 7, a polynucleotide comprising the sequence of SEQ ID NO 8, a polynucleotide comprising the sequence of SEQ ID NO 9, a polynucleotide comprising the sequence of SEQ ID NO 10, a polynucleotide comprising the sequence of SEQ ID NO 11 and a polynucleotide comprising the sequence of SEQ ID NO 12.

In any of the embodiments described herein, the compositions/kits may further comprise a sample. The sample may be a sample from a patient or a biological sample. Preferably, the sample is a sample which has been taken from a human subject, optionally wherein the human subject is a patient, optionally wherein the patient has or is suspected of having a viral infection. Most preferably, the sample is a sample taken from a patient having or suspected of having Zika virus infection. In some embodiments, the sample taken from a patient (i.e. a patient sample) is urine, blood, saliva or cells. Alternatively, in some embodiments the sample is a biological sample, preferably insect cells. Most preferably, when the sample is a biological sample, the sample is mosquito cells or tick cells.

### Compositions or Kits Comprising the Probes of the Invention

The present invention provides a composition or kit comprising a plurality of probes as described herein. A composition or kit comprising a plurality of probes at least one of which is according to the invention as described herein are contemplated. Compositions/kits comprising a plurality of any of the probes of the invention are also contemplated. In a most preferred embodiment, the kits of the invention comprise the compositions of the invention. Preferably, the compositions of the invention are solutions, suspensions, liquids or mixtures. The probes of the invention may comprise one type of polynucleotide, (i.e., the plurality of polynucleotides conjugated to the probe have the same sequence) or a plurality of different types of polynucleotides (i.e., the particle of the probe is conjugated to polynucleotides having a plurality of different sequences). In preferred aspects the probes of the invention comprise one type of polynucleotide, (i.e., the plurality of polynucleotides conjugated to the probe have the same sequence). The compositions or kits of the invention may comprise a plurality of one type of probe (i.e. a plurality of probes all comprising the same type of polynucleotide). The compositions or kits of the invention may comprise a plurality of different types of probe. The compositions or kits may comprise a plurality of one type of probe of the invention that comprises particles functionalised with (conjugated to) polynucleotides of the invention comprising one of the sequences described herein. Alternatively, the compositions or kits may comprise a plurality of different probes of the invention that comprise particles functionalised with (conjugated to) polynucleotides of the invention comprising different sequences described herein.

In some embodiments the compositions/kits may comprise a probe of the invention comprising a particle and a nucleotide wherein the nucleotide comprises the sequence of SEQ ID NO 6. In some embodiments the compositions/kits may comprise a probe of the invention comprising a particle and a nucleotide wherein the nucleotide comprises the sequence of SEQ ID NO 7. In some embodiments the compositions/kits may comprise a probe of the invention comprising a particle and a nucleotide wherein the nucleotide comprises the sequence of SEQ ID NO 8. In some embodiments the compositions/kits may comprise a probe of the invention comprising a particle and a nucleotide wherein the nucleotide comprises the sequence of SEQ ID NO 9. In some embodiments the compositions/kits may comprise a probe of the invention comprising a particle and a nucleotide wherein the nucleotide comprises the sequence of SEQ ID NO 10. In some embodiments the compositions/kits may comprise a probe of the invention comprising a particle and a nucleotide wherein the nucleotide comprises the sequence of SEQ ID NO 11.

In some embodiments the compositions/kits may comprise a probe of the invention comprising a particle and a nucleotide wherein the nucleotide comprises the sequence of SEQ ID NO 12. In some embodiments, any one of these compositions/kits may further comprise: a probe of the invention comprising a particle and a nucleotide wherein the nucleotide comprises the sequence of SEQ ID NO 6 and/or a probe of the invention comprising a particle and a nucleotide wherein the nucleotide comprises the sequence of SEQ ID NO 7 and/or a probe of the invention comprising a particle and a nucleotide wherein the nucleotide comprises the sequence of SEQ ID NO 8 and/or a probe of the invention comprising a particle and a nucleotide wherein the nucleotide comprises the sequence of SEQ ID NO 9 and/or a probe of the invention comprising a particle and a nucleotide wherein the nucleotide comprises the sequence of SEQ ID NO 10 and/or a probe of the invention comprising a particle and a nucleotide wherein the nucleotide comprises the sequence of SEQ ID NO 11 and/or a probe of the invention comprising a particle and a nucleotide wherein the nucleotide comprises the sequence of SEQ ID NO 12.

In any of the embodiments described herein, the compositions/kits may further comprise a sample. The sample may be a sample from a patient or a biological sample. Preferably, the sample is a sample which has been taken from a human subject, optionally wherein the human subject is a patient. In some aspects the patient has or is suspected of having a disease or condition such as bacterial or viral infection or cancer, optionally wherein the patient has or is suspected of having a bacterial or viral infection, preferably a viral infection. Most preferably, the sample is a sample taken from a patient having or suspected of having Zika virus infection. In some embodiments, the sample taken from a patient (i.e. a patient sample) is urine, blood, saliva or cells. Alternatively, in some embodiments the sample is a biological sample, preferably insect cells. Most preferably, when the sample is a biological sample, the sample is mosquito cells or tick cells.

In any of the embodiments described herein, the compositions/kits may further comprise a treated sample. The term “treated sample” may refer to a sample that has been taken from a patient and treated prior to use. Such treatment may include removal of cells, cell debris, proteins, fats, lipids, sugars. Such treatment preferably includes isolating the total nucleic acid content of the sample. Such treatment may include isolating the total nucleic acid content of the sample and amplifying or performing methods suitable for amplifying the target nucleic acid of interest, optionally wherein said target nucleic acid is associated with or diagnostic for a particular disease or condition. Preferably, the target nucleic acid is associated with or diagnostic for bacterial infection, viral infection or cancer, preferably bacterial or viral infection. Such treatment may include isolating the total nucleic acid content of the sample and amplifying or performing methods suitable for amplifying virus nucleic acids. In particular the virus nucleic acids may be a virus nucleic acid or a portion of a virus nucleic acid with the sequence according to any one of SEQ ID NO 1, SEQ ID NO 2, SEQ ID NO 3, SEQ ID NO 4, SEQ ID NO 5, SEQ ID NO 13, SEQ ID NO 14, SEQ ID NO 15, SEQ ID NO 16 or SEQ ID NO 17; preferably SEQ ID NO 5; most preferably any one of SEQ ID NO 13, SEQ ID NO 14, SEQ ID NO 15, SEQ ID NO 16 or SEQ ID NO 17. Amplification may be performed by methods well known in the art, for example, amplification may be performed by PCR, RT-PCR, rRT-PCR, NASBA, LAMP or HCR. Amplification may be performed by PCR or LAMP. Preferably, amplification is performed by PCR.

The compositions comprising the probes of the invention may preferably be solutions, suspensions, liquids or mixtures. Compositions/kits comprising the probes of the invention, wherein the particles are gold, are red in colour and show strong absorbance of light with a wavelength of 525 nm due to their surface plasmon resonance (G. Doria, et al., Sensors, 2012, 12:1657-1687). Aggregation of the gold particles alters the surface plasmon resonance and results in a shift in the absorbance to wavelengths between 600 to 700 nm. Compositions/kits comprising aggregated probes therefore are blue in colour. Increasing the salt concentration of a composition comprising the probes of the invention will cause aggregation of the probes resulting in a colour change from red to blue. However, if the polynucleotides of the probes are hybridised to a nucleic acid sequence, preferably the target nucleic acid of interest, then this protects the probes from aggregation when the salt concentration of the composition is increased and the solution remains red.

The compositions/kits comprising the probes of the invention as described herein may comprise the probes of the invention wherein a polynucleotide, preferably a plurality of polynucleotides, of the probes is hybridised to a target nucleic acid of interest as described herein, optionally wherein the nucleotide of the probe is hybridised to a virus nucleic acid. The compositions/kits comprising the probes of the invention as described herein may comprise the probes of the invention wherein a polynucleotide, preferably a plurality of polynucleotides, of the probe is not hybridised to a target nucleic acid of interest, optionally wherein the nucleotide of the probe is not hybridised to a virus nucleic acid.

The compositions/kits comprising the probes of the invention as described herein may comprise non-aggregated probes of the invention or aggregated probes of the invention. Such compositions/kits may further comprise a target nucleic acid of interest, preferably a (portion of a) virus nucleic acid. In some aspects, the compositions/kits comprising the probes of the invention as described herein comprise non-aggregated probes of the invention when the target nucleic acid of interest is added. Thus when the target nucleic acid of interest, which may be a virus nucleic acid, is present in the composition of the invention the nucleotides of the invention hybridise to the target nucleic acid of interest, which may be a virus nucleic acid, and upon increasing the salt concentration the probes of the invention do not aggregate and the composition of the invention is red in colour. Thus when the target nucleic acid of interest, which may be a virus nucleic acid, is not present in the composition of the invention the nucleotides of the invention do not hybridise to the target nucleic acid of interest, which may be a virus nucleic acid, and upon increasing the salt concentration the probes of the invention aggregate and the composition of the invention is blue in colour. Aggregated in this context should be understood to mean any aggregation that results in a colour change from substantially red to substantially blue. Non-aggregation in this context should be understood to mean where the aggregation is not sufficient to result in a colour change and the composition colour remains red.

Kits of the invention may comprise any of the compositions described herein.

The polynucleotides, probes or compositions of the present invention may be useful in diagnosing a disease or condition, such as bacterial infection, viral infection or cancer. The polynucleotides, probes or compositions of the present invention may also be useful in treating a disease or condition, such as bacterial infection, viral infection or cancer. In one aspect, the polynucleotides, probes or compositions of the present invention may also be useful in the treatment of Zika virus infection in a patient. Thus, the present invention also provides a pharmaceutical composition comprising a polynucleotide of the invention or a probe of the invention. The composition may further comprise a pharmaceutically acceptable carrier or excipient. The composition may be formulated for routes of administration including parenteral, intravenous, intradermal, subcutaneous, intraperitoneal, intramuscular, oral (e.g., inhalation), transdermal (topical) and transmucosal.

### Methods for Producing the Probes

The present invention also provides a method for preparing probes suitable for use in colorimetric methods for detecting a target nucleic acid. In some embodiments, the probes comprise a polynucleotide as described herein and are suitable for use in the colorimetric methods described herein for detecting a target viral nucleic acid of a Flaviviridae family virus, preferably a Zika virus RNA sequence.

The present invention comprises a method for preparing a probe comprising: (a) contacting a particle with a polynucleotide that is capable of hybridising to (i.e., binding to) a target nucleic acid of interest, optionally wherein the polynucleotide is complementary to a sequence within the target nucleic acid; and (b) contacting the particle and the polynucleotide with a salt source such that the salt concentration increases stepwise. The increasing salt concentration results in the polynucleotide binding to the particle. The methods may also utilise a plurality of particles and plurality of polynucleotides. Thus, the present invention comprises a method for preparing a probe comprising: (a) contacting a particle with a polynucleotide that hybridises to (i.e., binds to and optionally is complementary to) a sequence within a target nucleic acid, optionally to bind to the particle with the polynucleotide; and (b) contacting the particle and the polynucleotide with a salt source, wherein the salt concentration increases stepwise. The present invention also provides a probe produced by the methods described herein.

The particle may comprise or consist of metal. The particle may comprise or consist of gold, silver, a gold and silver alloy, or an alloy of gold with another metal, preferably the particle comprises or consists of gold. The particle is typically substantially spherical. The particle may have a diameter of between 1-100 nm, 5-80 nm, 10-50 nm, 10-40 nm, 12-48 nm, 10-30 nm, or 14-17 nm; preferably the particle has a diameter between 14-17 nm. The method typically results in a probe comprising a particle and a plurality of polynucleotides. Typically, the density of polynucleotides on (i.e., conjugated to) the particle is between 1-100 pmol/cm², 10-80 pmol/cm², 15-50 pmol/cm², 20-40 pmol/cm² or preferably between 20-30 pmol/cm². Typically, the number of copies of the polynucleotide on (i.e., conjugated to) the particle is between 10-400, 20-350, 50-300, 100-200, or preferably 140-180 copies.

The polynucleotide is typically single-stranded and between 5-100, 10-75, 10-50, or preferably between 10-30 nucleotides in length. The polynucleotide may comprise or consist of DNA, RNA or DNA and RNA, preferably DNA. The polynucleotide may further comprise a linker at the 3′ and/or 5′ end of the polynucleotide. The may comprise a linker and the linker may comprise nucleotides, optionally between 1-10, preferably between 1-5 nucleotides. Typically, the length of the polynucleotide does not include the nucleotides of the linker. The length of the polynucleotide typically describes the nucleotides that hybridise to the target nucleic acid sequence. The polynucleotide (optionally including the linker) preferably comprises a thiol group at the 3′ and/or 5′ end. The thiol group allows for conjugation to the metal, preferably gold, particle.

The target nucleic acid sequence may comprise DNA or RNA, preferably RNA. The target nucleic acid may be any length, but will comprise a sequence that hybridises to (binds to/is complementary to) the polynucleotide. The target nucleic acid may be associated with a disease phenotype. The target nucleic acid may be associated with or diagnostic of a disease or condition, such as a bacterial infection, a viral infection or cancer. The target nucleic acid may be isolated from a patient or subject suspected of having or having a disease. The target nucleic acid may be comprised within a sample isolated from a patient or subject suspected of having or having the particular disease or condition. The target nucleic acid may be a bacterial nucleic acid, a viral nucleic acid or a marker of cancer. The target nucleic acid may be a viral nucleic acid. In some preferred embodiments the target nucleic acid is a Flavividae virus RNA, preferably a Zika virus RNA, for example as described herein.

In some embodiments, the contacting occurs in a liquid. Typically the ratio of particle:polynucleotide (w/w) is 1:1-500:1, 2:1-450:1, 5:1-400:1, 10:1-350:1, 50:1-300:1, 100:1-250:1, or preferably 150:1-250:1. The salt source may comprise a concentrated salt solution. In some embodiments, the method comprises stepwise addition of the concentrated salt solution to the mixture of the particle and the polynucleotide to effect a gradual increase in the salt concentration, resulting in conjugation of the desired number of copies of the polynucleotide on the particle. Stepwise addition is described later herein.

In some embodiments, the method comprises: a) contacting a particle with a polynucleotide of the invention, optionally to bind the particle with the polynucleotide; and, b) increasing the salt concentration of the composition comprising the particle and the polynucleotide stepwise. The present invention also provides a probe produced by the methods described herein.

The density of polynucleotides conjugated to the particle in the probes of the invention is typically between 1-100 pmol/cm², 10-80 pmol/cm², 15-50 pmol/cm², 20-40 pmol/cm² or 20-30 pmol/cm²; preferably 20-30 pmol/cm². The number of polynucleotides conjugated to the particle in the probes of the invention is typically 10-400, 20-350, 50-300, 100-200, or preferably 140-180 copies.

In some embodiments, the method further comprises a step, prior to step (a), of determining the concentration of a solution of the polynucleotides of the invention. Suitable methods for determining the concentration of a solution of polynucleotides are well known in the art, but include, for example, using a spectrophotometer and measuring the absorbance at 260 nm. In some embodiments, step (a) of the method further comprises contacting (preferably by mixing) the polynucleotides with the particles (preferably gold particles) at a ratio of 1:1, 2:1, 5:1, 10:1, 50:1, 100:1, 150:1, 200:1, 250:1, 300:1 or 500:1. In some embodiments, step (a) of the method further comprises contacting (preferably by mixing) the polynucleotides with the particles (preferably gold particles) at a ratio of between 1:1-500:1, 2:1-450:1, 5:1-400:1, 10:1-350:1, 50:1-300:1, 100:1-250:1, or 150:1-250:1; preferably between 150:1-250:1. Step (a) of the method may further comprise contacting (preferably by mixing) the polynucleotides with the particles (preferably gold particles) at a ratio of 1:1 to 500:1, 2:1 to 450:1, 5:1 to 400:1, 10:1 to 350:1, 50:1 to 300:1, 100:1 to 250:1, or 150:1 to 250:1; preferably between 150:1 to 250:1. In some embodiments, the resulting composition comprising the particle and the polynucleotide further comprises a buffered solution. Preferably the buffered solution comprises phosphate, optionally the buffered solution comprises 10 mM phosphate buffer. The pH of the composition comprising the particle and the polynucleotide may be between 4-11, 5-10, 6-9, or 7-8; preferably pH 7-8 and most preferably about pH 8. The composition comprising the particle and the polynucleotide may further comprise sodium dodecyl sulphate (SDS). The final concentration of SDS is typically between 0.005-1%; 0.005-0.5%; 0.005-0.2%; 0.005-0.1%; 0.01-0.2%; 0.01-0.1%; 0.01-0.05%; 0.01-0.02% or 0.008-0.02%; or optionally, at least 0.001%, 0.002%, 0.003%, 0.004%, 0.005%, 0.01%, 0.02%, 0.03%, 0.04% or at least 0.05% SDS; preferably the concentration of SDS is between 0.008-0.02% and most preferably about 0.01%.

In some embodiments, step (b) of the method (increasing the salt concentration) further comprises either a gradual stepwise increase in salt concentration or a gradual continuous increase in salt concentration. In some preferred embodiments, step (b) further comprises increasing the salt concentration stepwise. Suitable steps in a stepwise gradient for increasing the salt concentration would be known to the skilled person. Typically, incremental steps in the stepwise gradient may result in a 0.05 to 0.2 M, i.e., a between 0.05-0.2 M increase in salt concentration. A suitable number of steps in the stepwise gradient could be easily selected by the skilled person. Typically there may be between 5-20 steps, 8-12 steps and preferably about 10 steps in the stepwise gradient. Thus, in some embodiments the salt concentration may increase from between 0-0.05 M salt to at least 0.05 M salt, to at least 0.1 M salt, to at least 0.2 M salt, to at least 0.3 M salt, to at least 0.4 M salt to at least 0.5 M salt to at least 0.6 M salt to at least 0.7 M salt to at least 0.8 M salt to at least 0.9 M salt to at least 1.0 M salt in the stepwise gradient. In a preferred embodiment, the salt concentration is increased in a first increment of between 0.05-0.1 M, then in increments of between 0.1-0.2 M over a period of time, up to a final concentration of salt of at least 0.3 M; at least 0.5 M; at least 0.6 M; at least 0.7 M; at least 0.8 M; at least 0.9 M or at least 1 M. In some embodiments, the time interval between each step in the stepwise gradient is between 5-60 minutes, 10-40 minutes, 15-30 minutes; preferably the time interval between each step in the stepwise gradient is at least 15 minutes and most preferably about 20 minutes. Optionally, the composition comprising the particle and the polynucleotide may be sonicated for up to 1 minutes; up to 5 minutes; up to 8 minutes; up to 10 minutes or up to 15 minutes after each step increasing the salt concentration in the stepwise gradient. In some embodiments step (b) of the method, increasing the salt concentration, is performed over up to 48 hours, up to 24 hours; up to 16 hours; up to 12 hours; up to 8 hours, up to 6 hours, or up to 4 hours; preferably up to 4 hours.

In some embodiments, the salt concentration is increased in step (b) of the method by contacting the composition comprising the particle and the polynucleotide with a salt source. Preferably, the salt source is a concentrated salt solution. The concentrated salt solution may have a concentration between 0.1-10 M, 0.2-5.0 M, 0.5-2.5 M or preferably 1.0 M to 2.0 M, i.e., between 1.0-2.0 M. The salt may be NaCl, MgCl₂, NiCl₂, NaBr, ZnCl₂, MnCl₂, BrCl, CdCl₂, CaCl₂, CoCl₂, CoCl₃, CuCl₂, CuCl, PbCl₂, PtCl₂, PtCl₄, KCl, RbCl, AgCl, SnCl₂, BrF, LiBr, KBr, AgBr, NaNO₂, Na₃PO₄, NaHPO₄, NaH₂PO₄, KH₂PO₄ or K₂HPO₄, or any other ionic halide. Preferably, the salt is either MgCL₂ or NaCl, most preferably NaCl. Preferably, the concentrated salt solution further comprises a buffer, most preferably phosphate buffer. In a preferred embodiment the salt solution further comprises 10 mM phosphate. The concentrated salt solution may further comprise sodium dodecyl sulphate (SDS). The final concentration of SDS is typically between 0.005-1%; 0.005-0.5%; 0.005-0.2%; 0.005-0.1%; 0.01-0.2%; 0.01-0.1%; 0.01-0.05%; 0.01-0.02% or 0.008-0.02%; preferably the concentration of SDS is between 0.008-0.02% and most preferably about 0.01%. In a preferred embodiment, the concentrated salt solution comprises 1.5 M NaCl, 10 MM phosphate pH 7-8 and 0.01% SDS.

In some embodiments, the method further comprises a step after step (b) wherein the resulting probes (comprising particles functionalised with polynucleotides) are incubated over night at room temperature in the presence of at least 0.6 M, 0.7 M, 0.8 M, 0.9 M or at least 1.0 M salt, preferably between 0.7-1.0 M salt. In some embodiments, the method further comprises a step after step (b) wherein after being incubated over night at room temperature, the probes are washed at least twice, at least three times, at least four times or at least five times, preferably twice, in phosphate buffer with an SDS concentration of between 0.005-1%; 0.005-0.5%; 0.005-0.2%; 0.005-0.1%; 0.01-0.2%; 0.01-0.1%; 0.01-0.05%; 0.01-0.02% or 0.008-0.02%; preferably the concentration of SDS is between 0.008-0.02%.

The present invention also provides an algorithm or a program for a computer, for use in producing the probes of the invention, wherein the program provides the user with the volume of the concentrated salt solution (between 1.0-2.0 M NaCl) required to:

a) increase the salt concentration of the composition comprising the particle and the polynucleotide to between 0.05-0.1 M;

b) subsequently increase the salt concentration of the composition comprising the particle and the polynucleotide by 0.1-0.2 M; and

c) subsequently increase the salt concentration of the composition comprising the particle and the polynucleotide according to step (b) between 5-10 times, such that the final salt concentration is between to 0.5-2.2 M, preferably between 0.6-1.0 M.

Thus, the user typically must input the following information into the algorithm:

a) the absorbance at 260 nm (OD260) of the solution of polynucleotides;

b) the volume of polynucleotides to be used (μl );

c) the ratio of particles:polynucleotides (between 1:150-1:250);

d) the concentration of the stock solution comprising the particles (nM);

e) the theoretical/provided absorbance at 260 nm of the solution of polynucleotides;

f) the theoretical/provided concentration of the solution of polynucleotides (nM).

The algorithm will then provide the following outputs:

1a) the volume of the stock solution comprising the particles to use (ml);

b) the volume of Solution 1 to add to the mixture of particles and polynucleotides (μl );

c) the volume of Solution 2 to add to the mixture (t1) between 5-10 times to increase the salt concentration in a stepwise gradient, wherein each step is between 0.1-0.2 M, wherein the final salt concentration is between 0.6-1.0 M;

wherein Solution 1 comprises phosphate buffer, pH 8.0 and between 1-3% SDS and Solution 2 comprises phosphate buffer, pH 8.0, between 1.0-2.0 M NaCl, and between 0.005-0.02% SDS.

This algorithm allows for the user to easily calculate the volumes of salt solution necessary to add at each stage in the method in order to increase the final salt concentration by the increments required by the method for preparing the probes of the invention. Using this algorithm ensures reproducibility of the synthesis of the probes for use in the methods of the invention. This algorithm also ensures that the probes that are produced according to the method above are optimised for use in the methods of the present invention for detecting target nucleic acids of interest, for example, for detecting nucleic acids from virus of the Flaviviridae family and the genus Flavivirus in biological or patient samples.

Use of the algorithm will result in producing probes which comprise particles (preferably gold particles) functionalised with (or conjugated to) polynucleotides, wherein the density of the polynucleotides on the particle surface is 5-50 pmol/cm², 10-40 pmol/cm² or 20-30 pmol/cm², preferably 20-30 pmol/cm². This density of polynucleotides on the particles provides the optimal critical density for use in the methods described herein for detecting target nucleic acids of interest, such as a Flaviviridae family virus or nucleic acid sequence thereof. One advantage of the algorithm is that the resulting probes with the particular polynucleotide density identified by the Inventors are highly sensitive and capable of providing a positive or negative result in the methods for detecting a Flaviviridae family virus or nucleic acid sequence thereof at a an assay salt concentration of only 25 mM salt, preferably NaCl or MgCl₂.

The algorithm may be incorporated into a computer program. The algorithm or computer program may be a program for a computer.

### Methods for Detecting Viral Nucleic Acids

The present invention provides a method of detecting a target nucleic acid of interest, preferably in a sample, the method comprising:

a) contacting a probe of the invention with a sample, to form a composition comprising the probe and the sample,

wherein the probe comprises a polynucleotide, or preferably a plurality of polynucleotides, capable of hybridising to (i.e., binding to), and optionally being complementary to, the target nucleic acid of interest; and

b) increasing the salt concentration of the composition from step a); and preferably,

c) detecting either
(i) a colour change if the target nucleic acid of interest is not present in the sample; or
(ii) no colour change if the target nucleic acid of interest is present in the sample.

In some aspects, the present invention provides a method of detecting a Flaviviridae family virus or nucleic acid sequence thereof, preferably in a sample, the method comprising:

a) contacting a probe or polynucleotide of the invention, specific for said virus or sequence with the sample;

b) increasing the salt concentration of a composition comprising the probe and the sample; and, preferably,

c) detecting either
(i) a colour change if the viral nucleic acid sequence is not present in the sample; or
(ii) no colour change if the viral nucleic acid sequence is present in the sample.

Any of the probes described herein, wherein the probes comprise any of the polynucleotides described herein, may be used in the methods of the invention for detecting a target nucleic acid of interest, such as a Flaviviridae family virus or nucleic acid sequence thereof in a sample.

The target nucleic acid of interest may be any nucleic acid that is associated with a particular disease or condition. For example, the target nucleic acid may be a diagnostic marker of a disease or condition or the presence of the target nucleic acid in a sample from a subject may be indicative of the subject having that disease or condition. The target nucleic acid of interest may comprise or consist of single or double stranded DNA or RNA. The target nucleic acid may be any length, for example the target nucleic acid may comprise or consist of 5, 10, 20, 30, 40, 50, 100, 200, 300, 400, 500, 1000, 2000, 3000, 4000, 5000, 8000 nucleotides, or 5-8000, 5-5000, 5-2000, 5-1000, 5-500, 5-400, 5-300, 5-200, 5-100, 5-80, 5-60, 5-50, 10-8000, 10-5000, 10-2000, 10-1000, 10-500, 10-400, 10-300, 10-200, 10-100, 10-80, 10-60, 10-50, 50-8000, 50-5000, 50-2000, 50-1000, 50-500, 50-400, 50-300, 50-200, 50-100, 100-8000, 100-5000, 100-2000, 100-1000, 100-500, preferably between 10-500 nucleotides in length. The target nucleic acid may be a bacterial nucleic acid, a viral nucleic acid, a nucleic acid from the subject, optionally comprising a mutation, a disease biomarker, a cancer biomarker, a nucleic acid from a foetus or a nucleic acid from a transplanted organ or tissue, preferably a bacterial nucleic acid, a viral nucleic acid. Accordingly, the target nucleic acid of interest may be any nucleic acid that is associated with a bacterial infection, a viral infection, cancer, pregnancy, or organ or tissue transplant rejection, preferably a bacterial infection or a viral infection.

In some embodiments the methods are for detecting a Flaviviridae family virus. The virus may be a virus of the Flaviviridae family and the genus Flavivirus. The virus may be selected from the group consisting of Zika virus, West Nile virus, dengue virus, tick-borne encephalitis virus, yellow fever virus, Japanese encephalitis virus, cell fusing agent virus (CFAV), Palm Creek virus (PCV), and Parramatta River virus (PaRV). In one preferred embodiment the virus is Zika virus.

In some preferred embodiments the methods are for detecting a nucleic acid sequence of or derived from a Flaviviridae family virus. In these preferred embodiments, the virus may be a virus of the Flaviviridae family and the genus Flavivirus. The virus may be selected from the group consisting of Zika virus, West Nile virus, dengue virus, tick-borne encephalitis virus, yellow fever virus, Japanese encephalitis virus, cell fusing agent virus (CFAV), Palm Creek virus (PCV), and Parramatta River virus (PaRV). In one preferred embodiment the virus is Zika virus. In some embodiments, the virus nucleic acid sequence is derived from, has been ejected from, is isolated from or removed from the virus.

The virus nucleic acid sequence may comprise single or double stranded DNA or RNA, preferably RNA, and more preferably single-stranded RNA. The virus nucleic acid sequence may consist of the virus genome, but typically the virus nucleic acid sequence comprises a portion of the virus genome. Thus, in some preferred embodiments, the nucleic acid sequence comprises at least part of the viral genome. The term “at least part of” a particular sequence may be understood to refer to the entirety of the particular sequence or a fragment of the particular sequence. Such fragments may comprise at least 10, 20, 30, 40, 50, 100, 200, 300, 400, 500, 1000, 2000, 3000, 4000, 5000, 8000 nucleotides of the particular sequence. Thus the virus nucleic acid sequence may comprise the virus genome or, for example, the virus nucleic acid sequence may comprise at least 10, 20, 30, 40, 50, 100, 200, 300, 400, 500, 1000, 2000, 3000, 4000, 5000, 8000 nucleotides of the virus genome.

In some embodiments, the nucleic acid sequence that is detected in the methods of the invention comprises at least part of the virus genome. In some embodiments, the nucleic acid sequence that is detected in the methods of the invention comprises (at least part of) the envelope gene of the virus. In some embodiments, the nucleic acid sequence that is detected in the methods of the invention comprises (at least part of) non-structural genes 1-5 of the virus. In some embodiments, the nucleic acid sequence that is detected in the methods of the invention comprises (at least part of) the non-structural gene 3 (NS3) of the virus. In some preferred embodiments, the nucleic acid sequence that is detected in the methods of the invention comprises (at least part of) the non-structural gene 5 (NS5) of the virus. In a further preferred embodiment, the nucleic acid sequence that is detected in the methods of the invention comprises (at least part of) the sequence between nucleotides 9182 and 9961 of the virus genome, which falls within the NS5 gene. In some particularly preferred embodiments, the nucleic acid sequence that is detected in the methods of the invention is derived from Zika virus. Thus in some embodiments, the nucleic acid sequence that is detected in the methods of the invention comprises at least part of SEQ ID NO 1 (the Zika virus genome). In some embodiments, the nucleic acid sequence that is detected in the methods of the invention comprises (at least part of) SEQ ID NO 2 (the envelope gene of Zika virus). In some embodiments, the nucleic acid sequence that is detected in the methods of the invention comprises (at least part of) SEQ ID NO 3 (the non-structural genes 1-5 of Zika virus). In some embodiments, the nucleic acid sequence that is detected in the methods of the invention comprises (at least part of) SEQ ID NO 4 (the NS3 gene of Zika virus). In some preferred embodiments, the nucleic acid sequence that is detected in the methods of the invention comprises (at least part of) SEQ ID NO 5 (the NS5 gene of Zika virus). In a further preferred embodiment, the nucleic acid sequence that is detected in the methods of the invention comprises (at least part of) the sequence between nucleotides 9182 and 9961 of SEQ ID NO 1.

In some embodiments, the nucleic acid sequence that is detected in the methods of the invention comprises a sequence that has at least 60%, 70%, 75%, 80%, 85%, 90%, 95%, 96%, 97%, 98%, 99% or at least 100% sequence identity to, preferably at least 80% sequence identity to, a sequence selected from (at least part of) SEQ ID NO 13, (at least part of) SEQ ID NO 14, (at least part of) SEQ ID NO 15, (at least part of) SEQ ID NO 16 or (at least part of) SEQ ID NO 17. In some embodiments, the nucleic acid sequence that is detected in the methods of the invention comprises a sequence that has at least 60%, 70%, 75%, 80%, 85%, 90%, 95%, 96%, 97%, 98%, 99% or at least 100% sequence identity to, preferably at least 80% sequence identity to (at least part of) SEQ ID NO 13. In some embodiments, the nucleic acid sequence that is detected in the methods of the invention comprises a sequence that has at least 60%, 70%, 75%, 80%, 85%, 90%, 95%, 96%, 97%, 98%, 99% or at least 100% sequence identity to, preferably at least 80% sequence identity to (at least part of) SEQ ID NO 14. In some embodiments, the nucleic acid sequence that is detected in the methods of the invention comprises a sequence that has at least 60%, 70%, 75%, 80%, 85%, 90%, 95%, 96%, 97%, 98%, 99% or at least 100% sequence identity to, preferably at least 80% sequence identity to (at least part of) SEQ ID NO 15. In some embodiments, the nucleic acid sequence that is detected in the methods of the invention comprises a sequence that has at least 60%, 70%, 75%, 80%, 85%, 90%, 95%, 96%, 97%, 98%, 99% or at least 100% sequence identity to, preferably at least 80% sequence identity to (at least part of) SEQ ID NO 16. In some embodiments, the nucleic acid sequence that is detected in the methods of the invention comprises a sequence that has at least 60%, 70%, 75%, 80%, 85%, 90%, 95%, 96%, 97%, 98%, 99% or at least 100% sequence identity to, preferably at least 80% sequence identity to (at least part of) SEQ ID NO 17. In some embodiments, the nucleic acid sequence that is detected in the methods of the invention comprises sequences that have at least 60%, 70%, 75%, 80%, 85%, 90%, 95%, 96%, 97%, 98%, 99% or at least 100% sequence identity to, preferably at least 80% sequence identity to, (at least part of) SEQ ID NO 13, (at least part of) SEQ ID NO 14, (at least part of) SEQ ID NO 15, (at least part of) SEQ ID NO 16 and (at least part of) SEQ ID NO 17.

In a most preferred embodiment, the nucleic acid sequence that is detected in the methods of the invention comprises (at least part of) at least one sequence selected from SEQ ID NO 13, SEQ ID NO 14, SEQ ID NO 15, SEQ ID NO 16 or SEQ ID NO 17. In some embodiments, the nucleic acid sequence that is detected in the methods of the invention comprises (at least part of) SEQ ID NO 13. In some embodiments, the nucleic acid sequence that is detected in the methods of the invention comprises (at least part of) SEQ ID NO 14. In some embodiments, the nucleic acid sequence that is detected in the methods of the invention comprises (at least part of) SEQ ID NO 15. In some embodiments, the nucleic acid sequence that is detected in the methods of the invention comprises (at least part of) SEQ ID NO 16. In some embodiments, the nucleic acid sequence that is detected in the methods of the invention comprises (at least part of) SEQ ID NO 17. In some embodiments, the nucleic acid sequence that is detected in the methods of the invention comprises (at least part of) SEQ ID NO 13, (at least part of) SEQ ID NO 14, (at least part of) SEQ ID NO 15, (at least part of) SEQ ID NO 16 and (at least part of) SEQ ID NO 17.

Any of the polynucleotides described herein may comprise a sequence that is capable of hybridising to (a portion of) the virus nucleic acid sequence detected in the methods of the invention. Thus, in some embodiments the nucleic acid sequence that is detected in the methods of the invention is capable of hybridising to at least one of SEQ ID NO 6, SEQ ID NO 7, SEQ ID NO 8, SEQ ID NO 9, SEQ ID NO 10, SEQ ID NO 11 or SEQ ID NO 12. In some embodiments the nucleic acid sequence that is detected in the methods of the invention is capable of hybridising to SEQ ID NO 6. In some embodiments the nucleic acid sequence that is detected in the methods of the invention is capable of hybridising to SEQ ID NO 7. In some embodiments the nucleic acid sequence that is detected in the methods of the invention is capable of hybridising to SEQ ID NO 8. In some embodiments the nucleic acid sequence that is detected in the methods of the invention is capable of hybridising to SEQ ID NO 9. In some embodiments the nucleic acid sequence that is detected in the methods of the invention is capable of hybridising to SEQ ID NO 10. In some embodiments the nucleic acid sequence that is detected in the methods of the invention is capable of hybridising to SEQ ID NO 11. In some embodiments the nucleic acid sequence that is detected in the methods of the invention is capable of hybridising to SEQ ID NO 12. In a preferred embodiment, the nucleic acid sequence that is detected in the methods of the invention is capable of hybridising to SEQ ID NO 6, SEQ ID NO 7, SEQ ID NO 8, SEQ ID NO 9, SEQ ID NO 10, SEQ ID NO 11 and SEQ ID NO 12.

Thus probes that are suitable for us in the methods of detecting a Flaviviridae family virus or nucleic acid sequence thereof, of the invention are any of the probes described herein, which may comprise any of the polynucleotides described herein. In a preferred embodiment of the invention, the probes for use in the methods of detecting a Flaviviridae family virus or nucleic acid sequence thereof comprise a particle, wherein the particle is gold, conjugated to a plurality of polynucleotides, wherein the polynucleotides comprise a sequence selected from SEQ ID NO 6, SEQ ID NO 7, SEQ ID NO 8, SEQ ID NO 9, SEQ ID NO 10, SEQ ID NO 11 or SEQ ID NO 12.

In some embodiments, the sample for use in the methods of the invention of detecting a target nucleic acid of interest, such as a Flaviviridae family virus or nucleic acid sequence thereof, may be a biological sample or a sample derived from/taken from a patient (i.e. a patient sample). Preferably, the sample is liquid. In some embodiments, the biological samples are insect cells, preferably mosquito cells or tick cells. In some embodiments, the sample derived from a patient is urine, saliva, blood or cells, preferably urine. In some embodiments, the biological or patient sample may be treated prior to use in the methods of the invention (i.e. prior to step (a) of the method). In samples containing cells, the cells may be lysed. In some embodiments, the total nucleic acid content of the sample may be purified or isolated. In some embodiments, the target nucleic acid of interest, such as virus nucleic acid sequences, preferably single-stranded RNA sequences, that may, or may not, be present in the sample and that therefore may, or may not be detected in the methods of the invention of detecting a target nucleic acid of interest, such as a Flaviviridae family virus or nucleic acid sequence thereof, may be amplified. Amplification may be performed by methods well known in the art, for example, amplification may be performed by PCR, RT-PCR, rRT-PCR, NASBA, LAMP or HCR, preferably the amplification may be performed by PCR or LAMP. Suitable primers for amplification of the target nucleic acid of interest, such as virus nucleic acid sequences, would be easily selected by the skilled person. This would be a routine matter and depends on the target nucleic acid of interest that is being tested for, such as for example on the virus that is being tested for. In preferred embodiments, the biological or patient sample is used directly in the methods of the invention of detecting a target nucleic acid of interest, such as a Flaviviridae family virus or nucleic acid sequence thereof.

Compositions comprising the probes of the invention as described herein for use in the methods of detecting a target nucleic acid of interest, such as a Flaviviridae family virus or nucleic acid sequence thereof, may be in liquid form, preferably a suspension. The concentration of probes in the composition may be between 0.5-50 nM; optionally between 0.8-20 nM; 1-10 nM; 1-5 nM; or 2-10 nM; preferably between 2-5 nM; optionally about 0.5, 1, 2, 3, 4, 5, 6, 7, 8, 9, 10, 15, 20, 25, 30, 40, or about 50 nM.

In some embodiments, contacting the probe and the sample comprises mixing the composition comprising the probe with the sample. Preferably, the composition comprising the probe is a liquid and the sample is a liquid. In some embodiments, the composition comprising the probes may be contacted with a solid support or matrix prior to contact with the sample. Thus the probes may be adhered to or dried on to the solid support or matrix. Thus, in some embodiments the composition comprising the probe may be a solid. Preferably, the sample is a liquid and is contacted (for example by spotting on to) with the solid composition comprising the probe.

In a preferred embodiment, the liquid composition comprising the probe is contacted with the liquid sample. In some embodiments the contacting occurs for between 1-60 minutes, 1-30 minutes, 1-20 minutes, 5-30 minutes, 10-30 minutes, 20-30 minutes or the contacting occurs for at least 1, 2, 3, 4, 5, 6, 7, 8, 9, 10, 15, 20, 25, 30, 40 or at least 60 minutes, preferably the contacting occurs for at least 15 minutes.

In some embodiments of the methods of detecting a target nucleic acid of interest, such as a Flaviviridae family virus or nucleic acid sequence thereof, increasing the salt concentration in step (b) is performed by contacting the composition comprising the probe and the sample with a salt source. Preferably, the salt source is a concentrated salt solution. In some embodiments the concentrated salt solution has a concentration between 5 mM-10 M, 50 mM-5 M, 100 mM-2.5 M, 200 mM-1 M or 200-500 mM; or at least 5 mM, 10 mM, 20 mM, 30 mM, 40 mM, 50 mM, 75 mM, 100 mM, 150 mM, 200 mM, 500 mM, 1 M, 2 M, 5 M or at least 10 M; preferably the concentrated salt solution has a concentration between 50 mM-5 M. The salt may be NaCl, MgCl₂, NiCl₂, NaBr, znCl₂, MnCl₂, BrCl, CdCl₂, CaCl₂, CoCl₂, CoCl₃, CuCl₂, CuCl, PbCl₂, PtCl₂, PtCl₄, KCl, RbCl, AgCl, SnCl₂, BrF, LiBr, KBr, AgBr, NaNO₂, Na₃PO₄, NaHPO₄, NaH₂PO₄, KH₂PO₄ or K₂HPO₄, or any other ionic halide. Preferably, the salt is either MgCL₂ or NaCl, most preferably NaCl. Preferably, the concentrated salt solution further comprises a buffer, most preferably phosphate buffer. In a preferred embodiment the salt solution further comprises 10 mM phosphate.

In some embodiments, the salt concentration of the composition comprising the probe and the sample is increased during step (b) rapidly or gradually, preferably rapidly, i.e. the salt concentration of the composition comprising the probe and the sample is increased during step (b) in less than 1 second, less than 2 seconds, less than 5 seconds, less than 10 seconds, more than 10 seconds, more than 20 seconds, more than 30 seconds, more than 1 minute, at least 10 minutes, at least 30 minutes or at least 1 hour, or over longer periods of time. Preferably, the salt concentration is increased in less than 5 seconds. In some embodiments, the salt concentration of the composition comprising the probe and the sample is increased during step (b) to a final salt concentration of 5 mM-10 M, 5 mM-5 M, 5 mM-1M, 5-500 mM, 10-500 mM, 10-250 mM, 10-150 mM, 10-100 mM, 10-50 mM, 15-40 mM or 20-30 mM; preferably the salt concentration of the composition comprising the probe and the sample is increased to a final salt concentration of between 10-50 mM and most optimally between 20-30 mM.

Compositions comprising the probes of the invention, produced by the methods for producing the probes of the invention as described herein, wherein the density of polynucleotides on the probes is 10-50 pmol/cm², preferably between 20-30 pmol/cm², typically will substantially aggregate when the polynucleotides of the probes are not hybridised to a target nucleic acid of interest, such as a virus nucleic acid, at a salt concentration of about 25 mM and typically will not substantially aggregate when the polynucleotides of the probes are hybridised to a target nucleic acid of interest, such as a virus nucleic acid, at a salt concentration of about 25 mM.

In some embodiments there is a period of time between performing steps (a) and (b) of the method. The period of time between performing steps (a) and (b) of the method may be between 1-60 minutes, 1-30 minutes, 1-20 minutes, 5-30 minutes, 10-30 minutes, 20-30 minutes or at least 1, 2, 3, 4, 5, 6, 7, 8, 9, 10, 15, 20, 25, 30, 40 or at least 60 minutes, preferably the period between performing steps (a) and (b) of the method is at least 15 minutes. In some embodiments there is a period of time between performing steps (b) and (c) of the method. The period of time between performing steps (b) and (c) of the method may be between 1-60 minutes, 1-30 minutes, 1-20 minutes, 5-30 minutes, 10-30 minutes, 20-30 minutes or at least 1, 2, 3, 4, 5, 6, 7, 8, 9, 10, 15, 20, 25, 30, 40 or at least 60 minutes, preferably the period between performing steps (a) and (b) of the method is at least 10 minutes. Optionally, step (a) may further comprise mixing the composition comprising the probe and the sample, i.e. by sonicating, vortexing or other means of mechanical agitation. Optionally, step (b) may further comprise sonicating the composition comprising the probe, the sample and a salt source as described herein, i.e. by sonicating, vortexing or other means of mechanical agitation. Suitable methods for sonicating, vortexing or performing other means of mechanical agitation are well known in the art. Furthermore, the skilled person could readily select alternative mixing procedures.

In some embodiments, the detecting in step (c) of the methods of the invention of detecting a target nucleic acid of interest, such as a Flaviviridae family virus or nucleic acid sequence thereof, comprises detecting the colour change or absence of a colour change by eye. Alternatively a spectrophotometer may be used to detect the colour change. The detecting step may involve analysis using a computer, for example, photographs of the result may be taken and uploaded onto a computer for analysis. Preferably, the colour change or no colour change is detected by eye, for example by comparison to a control. Such controls would be easily designed by a person skilled in the art, for example, such controls may include contacting a probes specific for said virus or sequence with a control sample, wherein the control sample comprises a buffered solution, such as 10 mM phosphate buffer, pH 7-8.

In some embodiments the colour change is from red or pink to blue, purple or colourless, preferably, the colour change is from red to blue. In some embodiments there is no colour change in the composition comprising the probe and the sample after increasing the salt concentration (i.e. the colour of the composition comprising the probe and the sample remains red), which indicates that the target nucleic acid of interest, such as a Flaviviridae family virus or nucleic acid sequence thereof, is present in the sample. Where the sample is a patient sample and there is no colour change in the composition indicating that the target nucleic acid of interest is present in the sample, this indicates that the patent has the disease or condition with which the target nucleic acid is associated. For example,here the sample is a patient sample, the nucleic acid of interest is a Flaviviridae family viral sequences and there is no colour change in the composition indicating that the viral sequences is present in the sample, this indicates that the patient is infected with said virus of the Flaviviridae family. In one preferred embodiment no colour change indicates Zika virus infection in the patient. In some embodiments there is a colour change in the composition comprising the probe and the sample after increasing the salt concentration (i.e. the colour of the composition comprising the probe and the sample turns blue), which indicates that the target nucleic acid sequence of interest, such as a Flaviviridae family virus or nucleic acid sequence thereof, is not present in the sample. Where the sample is a patient sample and there is a colour change in the composition, this indicates that the patent has the disease or condition with which the target nucleic acid is associated. For example, where the sample is a patient sample, and the target nucleic acid sequence of interest is a Flaviviridae family virus or nucleic acid sequence thereof, a colour change indicates that the patient is not infected with said virus of the Flaviviridae family. In one preferred embodiment a colour change indicates no Zika virus infection in the patient.

In some embodiments, the methods of the invention of detecting a target nucleic acid sequence of interest, such as a Flaviviridae family virus or nucleic acid sequence thereof, are performed at about room temperature, or the methods are performed at at least 0° C., 1° C., 5° C., 10° C., 15° C., 20° C., 22° C., 25° C., 28° C., 30° C., 35° C., 40° C., 50° C. or at least 60° C., preferably the methods are performed at between 20-28° C., most preferably at about room temperature (21-25 ° C.).

The polynucleotide sequence of the probe will hybridise to a complementary sequence in the target nucleic acid sequence of interest, such as a viral nucleic acid, if present. This will prevent the probes aggregating when the salt concentration increases and the solution remains a red colour due to the surface plasmon resonance of the non-aggregated gold particles. In the absence of a complementary target, the probes will aggregate when the salt concentration increases. This results in a shift in the surface plasmon resonance of the gold particles, which results in a colour change from red to blue.

### Kits for Use in the Detection Method

The present invention also provides a kit for use in the methods of the present invention of detecting a target nucleic acid of interest, preferably in a sample, the kit comprising:

a) a probe of the invention, wherein the probe is conjugated to a polynucleotide, or preferably a plurality of polynucleotides, that are capable of hybridising to (i.e., binding to), and optionally are complementary to, the target nucleic acid of interest; and optionally

b) a salt source.

The present invention also comprises a kit for use in the method of the present invention of detecting a Flaviviridae family virus or nucleic acid sequence thereof, preferably in a sample, the kit comprising:

a) a probe or polynucleotide of the invention, specific for a Flavividae family virus or nucleic acid sequence therefrom; and optionally

b) a salt source.

Any of the probes of the invention, comprising any of the polynucleotides of the invention described herein, may be included in such a kit.

The kit for use in the method of detecting a target nucleic acid of interest, such as a Flaviviridae family virus or nucleic acid sequence thereof, may comprise any probe described in the present disclosure. In some embodiments, the kit comprises a plurality of probes at least one of which is according to the invention as described herein. In some embodiments, the kit comprises a plurality of probes of the invention. In some embodiments the kit comprises a composition comprising the probe or a plurality of probes of the invention. In some embodiments the composition comprising the probe or a plurality of probes of the invention further comprises a buffer solution, for example phosphate buffers, HEPES buffers, Tris buffers, optionally wherein the pH is between 6-9, preferably between 7-8. In some embodiments the composition comprising the probe or a plurality of probes of the invention is a liquid, optionally a suspension. Where the composition comprising the probe or a plurality of probes of the invention is a liquid the concentration of the probes may be between 0.5 nM-1 M, 0.5 nM-500 mM, 0.5 nM-250 mM, 0.5 nM-100 mM, 0.5 nM-50 mM, 0.5 nM-10 mM, 0.5 nM-1 mM, 0.5-500 nM, 0.5-400 mM, 0.5-300 mM, 0.5-200 mM, 0.5-100 mM, 0.5-50 nM, 1-200 mM, 5-200 mM, 10-200 mM, 20-200 mM, 50-200 mM or 100-200 nM, preferably 0.5-50 nM. In some embodiments the composition comprising the probe or a plurality of probes of the invention is adhered to or dried onto a solid support or matrix.

In preferred embodiments, the probes included in the kit comprise a particle wherein the particle is gold. In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to a sequence selected from SEQ ID NO 6, SEQ ID NO 7, SEQ ID NO 8, SEQ ID NO 9, SEQ ID NO 10, SEQ ID NO 11 or SEQ ID NO 12. In some embodiments the kit comprises a plurality of probes, wherein each probe comprises at least one (preferably a plurality of) polynucleotide comprising a sequence with at least 60, 65, 70, 75, 80, 85, 90, 91, 92, 93, 94, 95, 96, 97, 98, 99 or 100% sequence identity, preferably at least 80% sequence identity, to a sequence selected from SEQ ID NO 6, SEQ ID NO 7, SEQ ID NO 8, SEQ ID NO 9, SEQ ID NO 10, SEQ ID NO 11 or SEQ ID NO 12, wherein each probe in the kit may comprise a different sequence.

In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to the sequence of SEQ ID NO 6. In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to the sequence of SEQ ID NO 7. In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to the sequence of SEQ ID NO 8. In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to the sequence of SEQ ID NO 9. In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to the sequence of SEQ ID NO 10. In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to the sequence of SEQ ID NO 11. In some embodiments the kit comprises at least one probe comprising a polynucleotide comprising a sequence with at least 60, 65, 70, 75, 80, 85, 90, 91, 92, 93, 94, 95, 96, 97, 98, 99 or 100% sequence identity, preferably at least 80% sequence identity, to the sequence of SEQ ID NO 12.

In some embodiments the kit comprises a probe comprising a polynucleotide comprising a sequence with at least 80% sequence identity to SEQ ID NO 6 and a probe comprising a polynucleotide comprising a sequence with at least 80% sequence identity to SEQ ID NO 7. In some embodiments the kit comprises a probe comprising a polynucleotide comprising a sequence with at least 80% sequence identity to SEQ ID NO 6 and a probe comprising a polynucleotide comprising a sequence with at least 80% sequence identity to SEQ ID NO 7 and a probe comprising a polynucleotide comprising a sequence with at least 80% sequence identity to SEQ ID NO 8. In some embodiments the kit comprises a probe comprising a polynucleotide comprising a sequence with at least 80% sequence identity to SEQ ID NO 6 and a probe comprising a polynucleotide comprising a sequence with at least 80% sequence identity to SEQ ID NO 7 and a probe comprising a polynucleotide comprising a sequence with at least 80% sequence identity to SEQ ID NO 8 and a probe comprising a polynucleotide comprising a sequence with at least 80% sequence identity to SEQ ID NO 9. In some embodiments the kit comprises a probe comprising a polynucleotide comprising a sequence with at least 80% sequence identity to SEQ ID NO 6 and a probe comprising a polynucleotide comprising a sequence with at least 80% sequence identity to SEQ ID NO 7 and a probe comprising a polynucleotide comprising a sequence with at least 80% sequence identity to SEQ ID NO 8 and a probe comprising a polynucleotide comprising a sequence with at least 80% sequence identity to SEQ ID NO 9 and a probe comprising a polynucleotide comprising a sequence with at least 80% sequence identity to SEQ ID NO 10. In some embodiments the kit comprises a probe comprising a polynucleotide comprising a sequence with at least 80% sequence identity to SEQ ID NO 6 and a probe comprising a polynucleotide comprising a sequence with at least 80% sequence identity to SEQ ID NO 7 and a probe comprising a polynucleotide comprising a sequence with at least 80% sequence identity to SEQ ID NO 8 and a probe comprising a polynucleotide comprising a sequence with at least 80% sequence identity to SEQ ID NO 9 and a probe comprising a polynucleotide comprising a sequence with at least 80% sequence identity to SEQ ID NO 10 and a probe comprising a polynucleotide comprising a sequence with at least 80% sequence identity to SEQ ID NO 11.

In a preferred embodiment, the kit comprises a probe comprising a polynucleotide comprising a sequence with at least 80% sequence identity to SEQ ID NO 6 and a probe comprising a polynucleotide comprising a sequence with at least 80% sequence identity to SEQ ID NO 7 and a probe comprising a polynucleotide comprising a sequence with at least 80% sequence identity to SEQ ID NO 8 and a probe comprising a polynucleotide comprising a sequence with at least 80% sequence identity to SEQ ID NO 9 and a probe comprising a polynucleotide comprising a sequence with at least 80% sequence identity to SEQ ID NO 10 and a probe comprising a polynucleotide comprising a sequence with at least 80% sequence identity to SEQ ID NO 11 and a probe comprising a polynucleotide comprising a sequence with at least 80% sequence identity to SEQ ID NO 12.

In preferred embodiments, the probes included in the kit comprise a particle wherein the particle is gold. In some embodiments the kit comprises at least one probe comprising a polynucleotide comprising a sequence selected from SEQ ID NO 6, SEQ ID NO 7, SEQ ID NO 8, SEQ ID NO 9, SEQ ID NO 10, SEQ ID NO 11 or SEQ ID NO 12. In some embodiments the kit comprises a plurality of probes, wherein each probe comprises at least one (preferably a plurality of) polynucleotide comprising a sequence selected from SEQ ID NO 6, SEQ ID NO 7, SEQ ID NO 8, SEQ ID NO 9, SEQ ID NO 10, SEQ ID NO 11 or SEQ ID NO 12, wherein each probe in the kit may comprise a different sequence.

In some embodiments the kit comprises at least one probe comprising a polynucleotide comprising a sequence of SEQ ID NO 6. In some embodiments the kit comprises at least one probe comprising a polynucleotide comprising a sequence of SEQ ID NO 7. In some embodiments the kit comprises at least one probe comprising a polynucleotide comprising a sequence of SEQ ID NO 8. In some embodiments the kit comprises at least one probe comprising a polynucleotide comprising a sequence of SEQ ID NO 9. In some embodiments the kit comprises at least one probe comprising a polynucleotide comprising a sequence of SEQ ID NO 10. In some embodiments the kit comprises at least one probe comprising a polynucleotide comprising a sequence of SEQ ID NO 11. In some embodiments the kit comprises at least one probe comprising a polynucleotide comprising a sequence of SEQ ID NO 12.

In some embodiments the kit comprises a probe comprising a polynucleotide comprising SEQ ID NO 6 and a probe comprising a polynucleotide comprising SEQ ID NO 7. In some embodiments the kit comprises a probe comprising a polynucleotide comprising SEQ ID NO 6 and a probe comprising a polynucleotide comprising SEQ ID NO 7 and a probe comprising a polynucleotide comprising SEQ ID NO 8. In some embodiments the kit comprises a probe comprising a polynucleotide comprising SEQ ID NO 6 and a probe comprising a polynucleotide comprising SEQ ID NO 7 and a probe comprising a polynucleotide comprising SEQ ID NO 8 and a probe comprising a polynucleotide comprising SEQ ID NO 9. In some embodiments the kit comprises a probe comprising a polynucleotide comprising SEQ ID NO 6 and a probe comprising a polynucleotide comprising SEQ ID NO 7 and a probe comprising a polynucleotide comprising SEQ ID NO 8 and a probe comprising a polynucleotide comprising SEQ ID NO 9 and a probe comprising a polynucleotide comprising SEQ ID NO 10. In some embodiments the kit comprises a probe comprising a polynucleotide comprising SEQ ID NO 6 and a probe comprising a polynucleotide comprising SEQ ID NO 7 and a probe comprising a polynucleotide comprising SEQ ID NO 8 and a probe comprising a polynucleotide comprising SEQ ID NO 9 and a probe comprising a polynucleotide comprising SEQ ID NO 10 and a probe comprising a polynucleotide comprising SEQ ID NO 11.

In a preferred embodiment, the kit comprises a probe comprising a polynucleotide comprising SEQ ID NO 6 and a probe comprising a polynucleotide comprising SEQ ID NO 7 and a probe comprising a polynucleotide comprising SEQ ID NO 8 and a probe comprising a polynucleotide comprising SEQ ID NO 9 and a probe comprising a polynucleotide comprising SEQ ID NO 10 and a probe comprising a polynucleotide comprising SEQ ID NO 11 and a probe comprising a polynucleotide comprising SEQ ID NO 12.

In a most preferred embodiment, the kit comprises a probe comprising a polynucleotide comprising SEQ ID NO 6 and/or a probe comprising a polynucleotide comprising SEQ ID NO 7 and/or a probe comprising a polynucleotide comprising SEQ ID NO 8 and/or a probe comprising a polynucleotide comprising SEQ ID NO 9 and/or a probe comprising a polynucleotide comprising SEQ ID NO 10 and/or a probe comprising a polynucleotide comprising SEQ ID NO 11 and/or a probe comprising a polynucleotide comprising SEQ ID NO 12.

In some embodiments the salt source is a concentrated salt solution. In some embodiments the concentrated salt solution has a concentration between 5 mM-10 M, 50 mM-5 M, 100 mM-2.5 M, 200 mM-1 M or 200-500 mM; or at least 5 mM, 10 mM, 20 mM, 30 mM, 40 mM, 50 mM, 75 mM, 100 mM, 150 mM, 200 mM, 500 mM, 1 M, 2 M, 5 M or at least 10 M; preferably the concentrated salt solution has a concentration between 50 mM-5 M. The salt may be NaCl, MgCl₂, NiCl₂, NaBr, ZnCl₂, MnCl₂, BrCl, CdCl₂, CaCl₂, CoCl₂, CoCl₃, CuCl₂, CuCl, PbCl₂, PtCl₂, PtCl₄, KCl, RbCl, AgCl, SnCl₂, BrF, LiBr, KBr, AgBr, NaNO₂, Na₃PO₄, NaHPO₄, NaH₂PO₄, KH₂PO₄ or K₂HPO₄, or any other ionic halide. Preferably, the salt is either MgCL₂ or NaCl, most preferably NaCl. Preferably, the concentrated salt solution further comprises a buffer, most preferably phosphate buffer. In a preferred embodiment the salt solution further comprises 10 mM phosphate.

The kit may further comprise a sample. The sample may be a sample from a patient or a biological sample. Preferably, the sample is a sample which has been taken from a human subject, optionally wherein the human subject is a patient, optionally wherein the patient has or is suspected of having a disease or condition associated with the target nucleic acid of interest, preferably a bacterial or a viral infection, most preferably a viral infection. Most preferably, the sample is a sample taken from a patient having or suspected of having Zika virus infection. In some embodiments, the sample taken from a patient (i.e. a patient sample) is urine, blood, saliva or cells. Alternatively, in some embodiments the sample is a biological sample, preferably insect cells. Most preferably, when the sample is a biological sample, the sample is mosquito cells or tick cells.

In some embodiments, the kit comprises a probe comprising a polynucleotide comprising a sequence that is hybridised to a target nucleic acid of interest, such as a virus nucleic acid sequence. In some embodiments, the kit comprises aggregated probes of the invention. In some embodiments, the kit comprises non-aggregated probes of the invention. In some embodiments, the kit comprises a composition comprising the probe, the salt source and a sample, wherein the colour of the composition is red. In some embodiments, the kit comprises a composition comprising the probe, the salt source and a sample, wherein the colour of the composition is blue. In some embodiments the kits described herein are for use in a method of diagnosing a disease or condition associated with the target nucleic acid of interest, optionally wherein the disease or condition is a bacterial infection, a viral infection or cancer, preferably a viral infection. In some embodiments the kits described herein are for use in a method of diagnosing Zika virus infection in a patient.

### Devices for Use in the Detection Method

Also disclosed herein is a device for use in detecting a target nucleic acid of interest in a sample, the device comprising:
a. a first chamber comprising a probe of the invention specific for the target nucleic acid of interest; and
b. a second chamber comprising a salt source, wherein: the device is configured to allow the probe to be contacted by the sample and, at a subsequent time, to allow the probe contacted by the sample to be contacted by the salt source.

Also disclosed herein is a device for use in detecting a Flaviviridae family virus or nucleic acid sequence thereof in a sample, the device comprising:
a. a first chamber comprising a probe specific for a Flavividae family virus or nucleic acid sequence therefrom; and
b. a second chamber comprising a salt source, wherein:

the device is configured to allow the probe to be contacted by the sample and, at a subsequent time, to allow the probe contacted by the sample to be contacted by the salt source.

In some embodiments the device is configured to allow the probe to be contacted in the first chamber by the sample. In some embodiments, the device is configured to allow a third chamber containing the sample to be detachably connected to the first chamber. In some embodiments, the device comprises a third chamber, the third chamber being configured to receive a sample. In some embodiments, the device comprises a third chamber, the third chamber comprising the sample. The sample may be derived from a patient. The sample may be liquid. The sample may be urine, blood, saliva, sweat, tears, or lymph, preferably urine. In some instances the sample comprises purified nucleic acids.

In some embodiments, the third chamber is configured to allow driving of at least a portion of the sample from the third chamber into the first chamber by increasing a pressure in the third chamber. In some embodiments, the device is configured to allow the pressure to be increased in the third chamber by reducing a volume of the third chamber. The third chamber may be a syringe and the first chamber may comprise an injection port. In some embodiments, the third chamber comprises at least one flexible wall and the reduction in volume is achieved via deformation of the flexible wall. The third chamber may be made from flexible plastic. The third chamber may comprise an injection port. The sample may be added to the third chamber through an injection port.

In some embodiments, the second chamber is configured to allow driving of at least a portion of the salt source from the second chamber into the first chamber by increasing a pressure in the second chamber. In some embodiments, the device is configured to allow the pressure to be increased in the second chamber by reducing a volume of the second chamber. The second chamber may be a syringe and the first chamber may comprise an injection port. In some embodiments, the second chamber comprises at least one flexible wall and the reduction in volume is achieved via deformation of the flexible wall. The second chamber may be made from flexible plastic. The second chamber may comprise an injection port. The salt source may be added to the second chamber through an injection port. In some embodiments the salt source is a concentrated salt solution. The salt solution may comprise a salt concentration of between 1.0 mM-20 M, 5 mM-10 M, 10 mM-5 M, 50 mM-1 M, 100 mM-500 mM, preferably between 0.05 M and 5 M. In some embodiments, the salt is an ionic halide, preferably sodium chloride (NaCl).

In some embodiments, the device comprises a one-way valve to allow venting of gas out of the first chamber to prevent reflux of material from the first chamber into the second chamber or, where provided, the third chamber.

In some embodiments, the first chamber is configured to allow optical inspection of material inside the first chamber through a wall of the first chamber. For example, the wall may comprise transparent plastic and/or glass. Typically, a wall of the first chamber will comprise a colourless, transparent material such that a colour change (i.e., from red to blue) may be observed in the first chamber.

The first chamber may comprise a probe of the invention. The first chamber may comprise one probe of the invention, more than one probe of the invention, or at least one probe of the invention. In some embodiments, the first chamber may comprise two or more probes of the invention or at least two probes of the invention. In some embodiments the first chamber may comprise a plurality of probes of the invention. For example, the first chamber may comprise at least 2, 3, 4, 5, 6, 7, 8, 9 or at least 10 probes of the invention. Typically, multiple copies of each probe of the invention will be present. In some embodiments, the first chamber will comprise a liquid, e.g., water or a buffered solution. The probes may be in solution or suspension within the first chamber. In some embodiments each probe of the invention will be present at the same concentration in the first chamber. Each probe of the invention in the first chamber may have a concentration of between 0.5-50 nM; optionally between 0.8-20 nM, 1-10 nM, 1-5 nM, or 2-10 nM; preferably between 2-5 nM.

In some embodiments, the probe is contacted with the sample in the first chamber of the device. The contacting may occur for at least 1, 2, 3, 4, 5 ,6, 7, 8, 9, 10, 15, 20, 25, 30, 35, 40, 45, 50, 55, or at least 60 minutes, preferably at least 5 minutes. In some embodiments the mixture of sample and probe may be heated to 95 ° C. for 5 minutes. In some embodiments, the mixture of the probe and sample is contacted with the salt source in the first chamber. Typically the mixture of the probe and sample is contacted with the salt source for at least 1, 2, 3, 4, 5 ,6, 7, 8, 9, 10, 15, 20, 25, 30, 35, 40, 45, 50, 55, or at least 60 minutes, preferably at least 5 minutes. Typically this contacting step is at room temperature.

In some aspects, the kit may comprise a composition comprising a probe or a plurality of compositions comprising different probes, preferably wherein each type of probe is in a separate container. For example, the different probes may be in different wells of an assay plate or in different tubes. The probes may be in solution or adhered to/dried onto a solid matrix or support. For example, a solution of probes may be soaked up onto a paper surface. The sample to be analysed could then be spotted or otherwise dispensed onto this paper surface. In some preferred aspects, both the salt source wherein the salt source is a concentrated salt solution and compositions comprising the probes, wherein the composition is a liquid, may be contained in eye-dropper bottles for ease of use and release of dose.

### EXAMPLES

### Example 1

### Synthesis of Gold Nanoparticles (AuNPs)

AuNPs with an average diameter of ˜13-14 nm were synthesized following the citrate reduction method described by Lee and Meisel (Lee and Meisel, 1982). Briefly, 250 mL of 1 mM HAuCl₄ was heated while stiffing in a 500 ml round-bottom flask. Then, 25 mL of 38.8 mM sodium citrate was added and the solution was refluxed for 30 min with continuous stirring. The solution was left to cool to room temperature and stored in the dark until further use.

Functionalization of AuNPs surface with thiolated ssDNA specific (Au-nanoprobe)

The probe sequences were designed using gene sequence database (GenBank) followed by in silico specificity assessment using BLAST tools for confirmation of specificity. For the experiments of Example 2 the probe sequence was 5′-GCAAACCTATCATC-3′ (ProbeZikaUniversal; SEQ ID NO 10). The ssDNA sequence was synthesis as thiol-modified at the 5′ end. The Au-nanoprobe was prepared by incubating the ssDNA thiol-modified oligonucleotides with AuNPs for 16 hours at room temperature with mild agitation. The solution was then washed several times with 10 mM phosphate buffer (pH 8) and increasing concentrations of NaCl until a final wash with 1.5 M of NaCl. The solution was finally centrifuged of 20 min at 14 500×g and washed then resuspended in 10 mM phosphate buffer (pH 8) and 0.1 M NaCl. The resulting Au-nanoprobes were stored in the dark at 4° C. until use.

Characterization of AuNPs and Au-nanoprobes was performed by transmission electron microscopy (TEM), UV-Vis and dynamic light scattering (DLS) (data not shown).

### Example 2

### Colorimetric Detection of Zika Virus RNA with Au-Nanoprobes

NATtrol™ Zika Virus External Run Controls are formulated with purified, intact virus particles that have been chemically modified to render them non-infectious and refrigerator stable. These controls are supplied in a purified protein matrix that mimics the composition of a true clinical specimen and are designed to evaluate the performance of nucleic acid tests for determination of the presence of Zika Virus RNA.

RNA was extracted from the ZeptoMetrix® NATtrol™ Zika Virus External Run Controls (Biomex GmbH, Germany) as a positive control and also from the human colorectal carcinoma cell line HCT116 (ATCC® CCL-247™; www.ATCC.org) as a negative control. RNA extraction from the HCT116 cell line was performed using the SV Total RNA Isolation System kit (Promega, Madison, Wis., USA). For the ZeptoMatrix® samples, RNA was extracted using the ZR Viral RNA Kit™ (Zymo Research, CA, USA) following heat inactivation at 65° C. for 5 minutes.

The resulting RNA extracts were dried and resuspended in Surine™ (Sigma-Aldrich), which is a certified reference analytical standard for use as a negative urine control.

The assay was performed in a total volume of 30 μL, containing a final concentration of 2.5 nM Au-nanoprobe in Surine™. The reaction mixture was heated up at 95° C. for 5 min and then cooled down to room-temperature for 5 min. A blank reaction was prepared in the exact same conditions but replacing the target by an equivalent volume of Surine™. The pre-determined salt (revelator) concentration was added to each reaction and after 15 min at room temperature, the colour of the solution evaluated. The mixtures and the blank were analysed by UV/Visible spectroscopy in a microplate reader (Tecan Infinte M200). Aggregation profiles were analysed in terms of the Ratio Abs_{525 nm}/Abs_{650 nm} (dispersed vs aggregated species) for the Au-nanoprobe.

Serial dilutions of the RNA extract were prepared in Surine™ to determine the limit of detection (LOD), which was found to be 10⁸ viral particles.

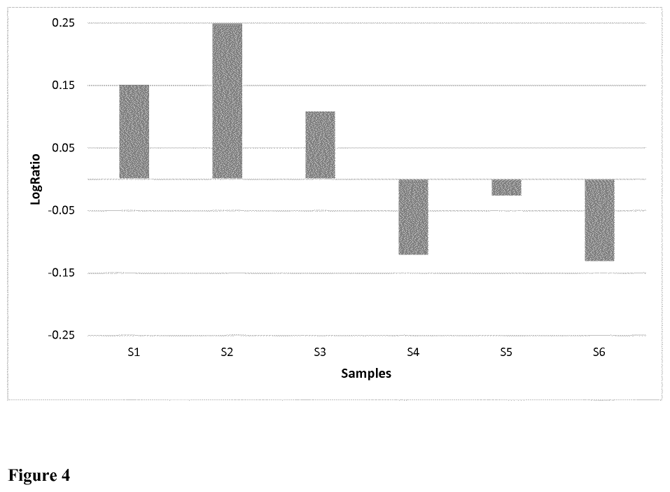

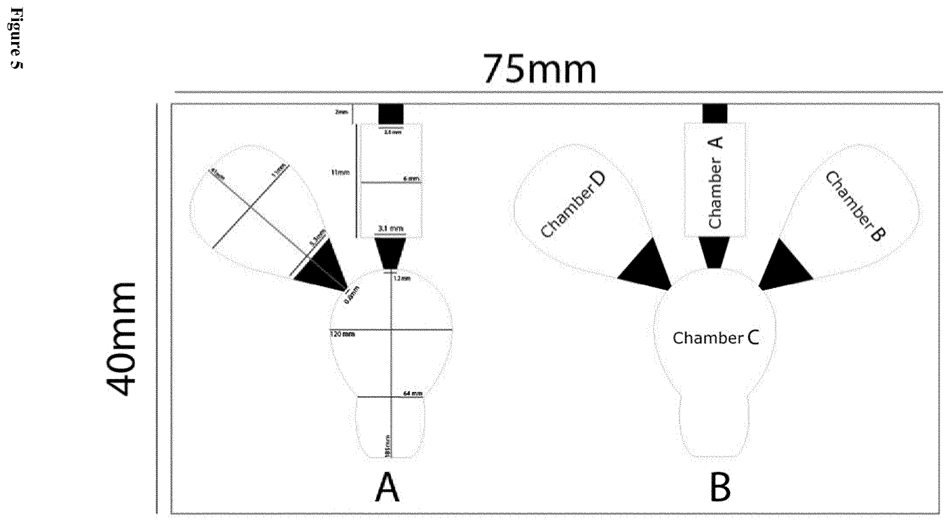

Samples S1-S3 containing the equivalent to 1-10×10⁹ viral particles, and Samples S4-S6 spiked with RNA from the control (i.e. human cells), were prepared in Surine™ and assayed under blind testing conditions. The results are shown in FIG. 4. Positive LogRatio represent positive samples (i.e. red solution). The colorimetric assay could sensitively and accurately detect the presence of Zika virus RNA in Surine™. The samples containing only RNA from the HCT116 cell line resulted in a negative LogRatio in the assay (i.e., blue solution) indicating that the assay specifically detects Zika virus RNA. These results demonstrate that the assay would be effective in detecting Zika virus in urine samples from patients and provide an easy to use point of care diagnostic test for Zika virus infection.

Informal Sequence Listing
SEQ ID NO 1: Zika virus (strain MR 766) complete genome
(ACC. Number AY 632535.2)
AGTTGTTGATCTGTGTGAGTCAGACTGCGACAGTTCGAGTCTGAAGCGAGAGCTAACAACAGTA
TCAACAGGTTTAATTTGGATTTGGAAACGAGAGTTTCTGGTCATGAAAAACCCCAAAGAAGAA
ATCCGGAGGATCCGGATTGTCAATATGCTAAAACGCGGAGTAGCCCGTGTAAACCCCTTGGGA
GGTTTGAAGAGGTTGCCAGCCGGACTTCTGCTGGGTCATGGACCCATCAGAATGGTTTTGGCGA
TACTAGCCTTTTTGAGATTTACAGCAATCAAGCCATCACTGGGCCTTATCAACAGATGGGGTTC
CGTGGGGAAAAAAGAGGCTATGGAAATAATAAAGAAGTTCAAGAAAGATCTTGCTGCCATGTT
GAGAATAATCAATGCTAGGAAAGAGAGGAAGAGACGTGGCGCAGACACCAGCATCGGAATCA
TTGGCCTCCTGCTGACTACAGCCATGGCAGCAGAGATCACTAGACGCGGGAGTGCATACTACAT
GTACTTGGATAGGAGCGATGCCGGGAAGGCCATTTCGTTTGCTACCACATTGGGAGTGAACAAG
TGCCACGTACAGATCATGGACCTCGGGCACATGTGTGACGCCACCATGAGTTATGAGTGCCCTA
TGCTGGATGAGGGAGTGGAACCAGATGATGTCGATTGCTGGTGCAACACGACATCAACTTGGG
TTGTGTACGGAACCTGTCATCACAAAAAAGGTGAGGCACGGCGATCTAGAAGAGCCGTGACGC
TCCCTTCTCACTCTACAAGGAAGTTGCAAACGCGGTCGCAGACCTGGTTAGAATCAAGAGAATA
CACGAAGCACTTGATCAAGGTTGAAAACTGGATATTCAGGAACCCCGGGTTTGCGCTAGTGGCC
GTTGCCATTGCCTGGCTTTTGGGAAGCTCGACGAGCCAAAAAGTCATATACTTGGTCATGATAC
TGCTGATTGCCCCGGCATACAGTATCAGGTGCATTGGAGTCAGCAATAGAGACTTCGTGGAGGG
CATGTCAGGTGGGACCTGGGTTGATGTTGTCTTGGAACATGGAGGCTGCGTTACCGTGATGGCA
CAGGACAAGCCAACAGTCGACATAGAGTTGGTCACGACGACGGTTAGTAACATGGCCGAGGTA
AGATCCTATTGCTACGAGGCATCGATATCGGACATGGCTTCGGACAGTCGTTGCCCAACACAAG
GTGAAGCCTACCTTGACAAGCAATCAGACACTCAATATGTCTGCAAAAGAACATTAGTGGACA
GAGGTTGGGGAAACGGTTGTGGACTTTTTGGCAAAGGGAGCTTGGTGACATGTGCCAAGTTTAC
GTGTTCTAAGAAGATGACCGGGAAGAGCATTCAACCGGAAAATCTGGAGTATCGGATAATGCT
ATCAGTGCATGGCTCCCAGCATAGCGGGATGATTGGATATGAAACTGACGAAGATAGAGCGAA
AGTCGAGGTTACGCCTAATTCACCAAGAGCGGAAGCAACCTTGGGAGGCTTTGGAAGCTTAGG
ACTTGACTGTGAACCAAGGACAGGCCTTGACTTTTCAGATCTGTATTACCTGACCATGAACAAT
AAGCATTGGTTGGTGCACAAAGAGTGGTTTCATGACATCCCATTGCCTTGGCATGCTGGGGCAG
ACACCGGAACTCCACACTGGAACAACAAAGAGGCATTGGTAGAATTCAAGGATGCCCACGCCA
AGAGGCAAACCGTCGTCGTTCTGGGGAGCCAGGAAGGAGCCGTTCACACGGCTCTCGCTGGAG
CTCTAGAGGCTGAGATGGATGGTGCAAAGGGAAGGCTGTTCTCTGGCCATTTGAAATGCCGCCT
AAAAATGGACAAGCTTAGATTGAAGGGCGTGTCATATTCCTTGTGCACTGCGGCATTCACATTC
ACCAAGGTCCCAGCTGAAACACTGCATGGAACAGTCACAGTGGAGGTGCAGTATGCAGGGACA
GATGGACCCTGCAAGATCCCAGTCCAGATGGCGGTGGACATGCAGACCCTGACCCCAGTTGGA
AGGCTGATAACCGCCAACCCCGTGATTACTGAAAGCACTGAGAACTCAAAGATGATGTTGGAG
CTTGACCCACCATTTGGGGATTCTTACATTGTCATAGGAGTTGGGGACAAGAAAATCACCCACC
ACTGGCATAGGAGTGGTAGCACCATCGGAAAGGCATTTGAGGCCACTGTGAGAGGCGCCAAGA
GAATGGCAGTCCTGGGGGATACAGCCTGGGACTTCGGATCAGTCGGGGGTGTGTTCAACTCACT
GGGTAAGGGCATTCACCAGATTTTTGGAGCAGCCTTCAAATCACTGTTTGGAGGAATGTCCTGG
TTCTCACAGATCCTCATAGGCACGCTGCTAGTGTGGTTAGGTTTGAACACAAAGAATGGATCTA
TCTCCCTCACATGCTTGGCCCTGGGGGGAGTGATGATCTTCCTCTCCACGGCTGTTTCTGCTGAC
GTGGGGTGCTCAGTGGACTTCTCAAAAAAGGAAACGAGATGTGGCACGGGGGTATTCATCTAT
AATGATGTTGAAGCCTGGAGGGACCGGTACAAGTACCATCCTGACTCCCCCCGCAGATTGGCAG
CAGCAGTCAAGCAGGCCTGGGAAGAGGGGATCTGTGGGATCTCATCCGTTTCAAGAATGGAAA
ACATCATGTGGAAATCAGTAGAAGGGGAGCTCAATGCTATCCTAGAGGAGAATGGAGTTCAAC
TGACAGTTGTTGTGGGATCTGTAAAAAACCCCATGTGGAGAGGTCCACAAAGATTGCCAGTGCC
TGTGAATGAGCTGCCCCATGGCTGGAAAGCCTGGGGGAAATCGTATTTTGTTAGGGCGGCAAA
GACCAACAACAGTTTTGTTGTCGACGGTGACACACTGAAGGAATGTCCGCTTGAGCACAGAGC
ATGGAATAGTTTTCTTGTGGAGGATCACGGGTTTGGAGTCTTCCACACCAGTGTCTGGCTTAAG
GTCAGAGAAGATTACTCATTAGAATGTGACCCAGCCGTCATAGGAACAGCTGTTAAGGGAAGG
GAGGCCGCGCACAGTGATCTGGGCTATTGGATTGAAAGTGAAAAGAATGACACATGGAGGCTG
AAGAGGGCCCACCTGATTGAGATGAAAACATGTGAATGGCCAAAGTCTCACACATTGTGGACA
GATGGAGTAGAAGAAAGTGATCTTATCATACCCAAGTCTTTAGCTGGTCCACTCAGCCACCACA
ACACCAGAGAGGGTTACAGAACCCAAGTGAAAGGGCCATGGCACAGTGAAGAGCTTGAAATCC
GGTTTGAGGAATGTCCAGGCACCAAGGTTTACGTGGAGGAGACATGCGGAACTAGAGGACCAT
CTCTGAGATCAACTACTGCAAGTGGAAGGGTCATTGAGGAATGGTGCTGTAGGGAATGCACAA
TGCCCCCACTATCGTTTCGAGCAAAAGACGGCTGCTGGTATGGAATGGAGATAAGGCCCAGGA
AAGAACCAGAGAGCAACTTAGTGAGGTCAATGGTGACAGCGGGGTCAACCGATCATATGGACC
ACTTCTCTCTTGGAGTGCTTGTGATTCTACTCATGGTGCAGGAGGGGTTGAAGAAGAGAATGAC
CACAAAGATCATCATGAGCACATCAATGGCAGTGCTGGTAGTCATGATCTTGGGAGGATTTTCA
ATGAGTGACCTGGCCAAGCTTGTGATCCTGATGGGTGCTACTTTCGCAGAAATGAACACTGGAG
GAGATGTAGCTCACTTGGCATTGGTAGCGGCATTTAAAGTCAGACCAGCCTTGCTGGTCTCCTT
CATTTTCAGAGCCAATTGGACACCCCGTGAGAGCATGCTGCTAGCCCTGGCTTCGTGTCTTCTGC
AAACTGCGATCTCTGCTCTTGAAGGTGACTTGATGGTCCTCATTAATGGATTTGCTTTGGCCTGG
TTGGCAATTCGAGCAATGGCCGTGCCACGCACTGACAACATCGCTCTACCAATCTTGGCTGCTC
TAACACCACTAGCTCGAGGCACACTGCTCGTGGCATGGAGAGCGGGCCTGGCTACTTGTGGAG
GGATCATGCTCCTCTCCCTGAAAGGGAAAGGTAGTGTGAAGAAGAACCTGCCATTTGTCATGGC
CCTGGGATTGACAGCTGTGAGGGTAGTAGACCCTATTAATGTGGTAGGACTACTGTTACTCACA
AGGAGTGGGAAGCGGAGCTGGCCCCCTAGTGAAGTTCTCACAGCCGTTGGCCTGATATGTGCAC
TGGCCGGAGGGTTTGCCAAGGCAGACATTGAGATGGCTGGACCCATGGCTGCAGTAGGCTTGCT
AATTGTCAGCTATGTGGTCTCGGGAAAGAGTGTGGACATGTACATTGAAAGAGCAGGTGACAT
CACATGGGAAAAGGACGCGGAAGTCACTGGAAACAGTCCTCGGCTTGACGTGGCACTGGATGA
GAGTGGTGACTTCTCCTTGGTAGAGGAAGATGGTCCACCCATGAGAGAGATCATACTCAAGGTG
GTCCTGATGGCCATCTGTGGCATGAACCCAATAGCTATACCTTTTGCTGCAGGAGCGTGGTATG
TGTATGTGAAGACTGGGAAAAGGAGTGGCGCCCTCTGGGACGTGCCTGCTCCCAAAGAAGTGA
AGAAAGGAGAGACCACAGATGGAGTGTACAGAGTGATGACTCGCAGACTGCTAGGTTCAACAC
AGGTTGGAGTGGGAGTCATGCAAGAGGGAGTCTTCCACACCATGTGGCACGTTACAAAAGGAG
CCGCACTGAGGAGCGGTGAGGGAAGACTTGATCCATACTGGGGGGATGTCAAGCAGGACTTGG
TGTCATACTGTGGGCCTTGGAAGTTGGATGCAGCTTGGGATGGACTCAGCGAGGTACAGCTTTT
GGCCGTACCTCCCGGAGAGAGGGCCAGAAACATTCAGACCCTGCCTGGAATATTCAAGACAAA
GGACGGGGACATCGGAGCAGTTGCTCTGGACTACCCTGCAGGGACCTCAGGATCTCCGATCCTA
GACAAATGTGGAAGAGTGATAGGACTCTATGGCAATGGGGTTGTGATCAAGAATGGAAGCTAT
GTTAGTGCTATAACCCAGGGAAAGAGGGAGGAGGAGACTCCGGTTGAATGTTTCGAACCCTCG
ATGCTGAAGAAGAAGCAGCTAACTGTCTTGGATCTGCATCCAGGAGCCGGAAAAACCAGGAGA
GTTCTTCCTGAAATAGTCCGTGAAGCCATAAAAAAGAGACTCCGGACAGTGATCTTGGCACCAA
CTAGGGTTGTCGCTGCTGAGATGGAGGAGGCCTTGAGAGGACTTCCGGTGCGTTACATGACAAC
AGCAGTCAACGTCACCCATTCTGGGACAGAAATCGTTGATTTGATGTGCCATGCCACTTTCACTT
CACGCTTACTACAACCCATCAGAGTCCCTAATTACAATCTCAACATCATGGATGAAGCCCACTT
CACAGACCCCTCAAGTATAGCTGCAAGAGGATACATATCAACAAGGGTTGAAATGGGCGAGGC
GGCTGCCATTTTTATGACTGCCACACCACCAGGAACCCGTGATGCGTTTCCTGACTCTAACTCAC
CAATCATGGACACAGAAGTGGAAGTCCCAGAGAGAGCCTGGAGCTCAGGCTTTGATTGGGTGA
CAGACCATTCTGGGAAAACAGTTTGGTTCGTTCCAAGCGTGAGAAACGGAAATGAAATCGCAG
CCTGTCTGACAAAGGCTGGAAAGCGGGTCATACAGCTCAGCAGGAAGACTTTTGAGACAGAAT
TTCAGAAAACAAAAAATCAAGAGTGGGACTTTGTCATAACAACTGACATCTCAGAGATGGGCG
CCAACTTCAAGGCTGACCGGGTCATAGACTCTAGGAGATGCCTAAAACCAGTCATACTTGATGG
TGAGAGAGTCATCTTGGCTGGGCCCATGCCTGTCACGCATGCTAGTGCTGCTCAGAGGAGAGGA
CGTATAGGCAGGAACCCTAACAAACCTGGAGATGAGTACATGTATGGAGGTGGGTGTGCAGAG
ACTGATGAAGGCCATGCACACTGGCTTGAAGCAAGAATGCTTCTTGACAACATCTACCTCCAGG
ATGGCCTCATAGCCTCGCTCTATCGGCCTGAGGCCGATAAGGTAGCCGCCATTGAGGGAGAGTT
TAAGCTGAGGACAGAGCAAAGGAAGACCTTCGTGGAACTCATGAAGAGAGGAGACCTTCCCGT
CTGGCTAGCCTATCAGGTTGCATCTGCCGGAATAACTTACACAGACAGAAGATGGTGCTTTGAT
GGCACAACCAACAACACCATAATGGAAGACAGTGTACCAGCAGAGGTTTGGACAAAGTATGGA
GAGAAGAGAGTGCTCAAACCGAGATGGATGGATGCTAGGGTCTGTTCAGACCATGCGGCCCTG
AAGTCGTTCAAAGAATTCGCCGCTGGAAAAAGAGGAGCGGCTTTGGGAGTAATGGAGGCCCTG
GGAACACTGCCAGGACACATGACAGAGAGGTTTCAGGAAGCCATTGACAACCTCGCCGTGCTC
ATGCGAGCAGAGACTGGAAGCAGGCCTTATAAGGCAGCGGCAGCCCAACTGCCGGAGACCCTA
GAGACCATTATGCTCTTAGGTTTGCTGGGAACAGTTTCACTGGGGATCTTCTTCGTCTTGATGCG
GAATAAGGGCATCGGGAAGATGGGCTTTGGAATGGTAACCCTTGGGGCCAGTGCATGGCTCAT
GTGGCTTTCGGAAATTGAACCAGCCAGAATTGCATGTGTCCTCATTGTTGTGTTTTTATTACTGG
TGGTGCTCATACCCGAGCCAGAGAAGCAAAGATCTCCCCAAGATAACCAGATGGCAATTATCA
TCATGGTGGCAGTGGGCCTTCTAGGTTTGATAACTGCAAACGAACTTGGATGGCTGGAAAGAAC
AAAAAATGACATAGCTCATCTAATGGGAAGGAGAGAAGAAGGAGCAACCATGGGATTCTCAAT
GGACATTGATCTGCGGCCAGCCTCCGCCTGGGCTATCTATGCCGCATTGACAACTCTCATCACC
CCAGCTGTCCAACATGCGGTAACCACTTCATACAACAACTACTCCTTAATGGCGATGGCCACAC
AAGCTGGAGTGCTGTTTGGCATGGGCAAAGGGATGCCATTTATGCATGGGGACCTTGGAGTCCC
GCTGCTAATGATGGGTTGCTATTCACAATTAACACCCCTGACTCTGATAGTAGCTATCATTCTGC
TTGTGGCGCACTACATGTACTTGATCCCAGGCCTACAAGCGGCAGCAGCGCGTGCTGCCCAGAA
AAGGACAGCAGCTGGCATCATGAAGAATCCCGTTGTGGATGGAATAGTGGTAACTGACATTGA
CACAATGACAATAGACCCCCAGGTGGAGAAGAAGATGGGACAAGTGTTACTCATAGCAGTAGC
CATCTCCAGTGCTGTGCTGCTGCGGACCGCCTGGGGATGGGGGGAGGCTGGAGCTCTGATCACA
GCAGCGACCTCCACCTTGTGGGAAGGCTCTCCAAACAAATACTGGAACTCCTCTACAGCCACCT
CACTGTGCAACATCTTCAGAGGAAGCTATCTGGCAGGAGCTTCCCTTATCTATACAGTGACGAG
AAACGCTGGCCTGGTTAAGAGACGTGGAGGTGGGACGGGAGAGACTCTGGGAGAGAAGTGGA
AAGCTCGTCTGAATCAGATGTCGGCCCTGGAGTTCTACTCTTATAAAAAGTCAGGTATCACTGA
AGTGTGTAGAGAGGAGGCTCGCCGTGCCCTCAAGGATGGAGTGGCCACAGGAGGACATGCCGT
ATCCCGGGGAAGTGCAAAGATCAGATGGTTGGAGGAGAGAGGATATCTGCAGCCCTATGGGAA
GGTTGTTGACCTCGGATGTGGCAGAGGGGGCTGGAGCTATTATGCCGCCACCATCCGCAAAGTG
CAGGAGGTGAGAGGATACACAAAGGGAGGTCCCGGTCATGAAGAACCCATGCTGGTGCAAAGC
TATGGGTGGAACATAGTTCGTCTCAAGAGTGGAGTGGACGTCTTCCACATGGCGGCTGAGCCGT
GTGACACTCTGCTGTGTGACATAGGTGAGTCATCATCTAGTCCTGAAGTGGAAGAGACACGAAC
ACTCAGAGTGCTCTCTATGGTGGGGGACTGGCTTGAAAAAAGACCAGGGGCCTTCTGTATAAAG
GTGCTGTGCCCATACACCAGCACTATGATGGAAACCATGGAGCGACTGCAACGTAGGCATGGG
GGAGGATTAGTCAGAGTGCCATTGTGTCGCAACTCCACACATGAGATGTACTGGGTCTCTGGGG
CAAAGAGCAACATCATAAAAAGTGTGTCCACCACAAGTCAGCTCCTCCTGGGACGCATGGATG
GCCCCAGGAGGCCAGTGAAATATGAGGAGGATGTGAACCTCGGCTCGGGTACACGAGCTGTGG
CAAGCTGTGCTGAGGCTCCTAACATGAAAATCATCGGCAGGCGCATTGAGAGAATCCGCAATG
AACATGCAGAAACATGGTTTCTTGATGAAAACCACCCATACAGGACATGGGCCTACCATGGGA
GCTACGAAGCCCCCACGCAAGGATCAGCGTCTTCCCTCGTGAACGGGGTTGTTAGACTCCTGTC
AAAGCCTTGGGACGTGGTGACTGGAGTTACAGGAATAGCCATGACTGACACCACACCATACGG
CCAACAAAGAGTCTTCAAAGAAAAAGTGGACACCAGGGTGCCAGATCCCCAAGAAGGCACTCG
CCAGGTAATGAACATAGTCTCTTCCTGGCTGTGGAAGGAGCTGGGGAAACGCAAGCGGCCACG
CGTCTGCACCAAAGAAGAGTTTATCAACAAGGTGCGCAGCAATGCAGCACTGGGAGCAATATT
TGAAGAGGAAAAAGAATGGAAGACGGCTGTGGAAGCTGTGAATGATCCAAGGTTTTGGGCCCT
AGTGGATAGGGAGAGAGAACACCACCTGAGAGGAGAGTGTCACAGCTGTGTGTACAACATGAT
GGGAAAAAGAGAAAAGAAGCAAGGAGAGTTCGGGAAAGCAAAAGGTAGCCGCGCCATCTGGT
ACATGTGGTTGGGAGCCAGATTCTTGGAGTTTGAAGCCCTTGGATTCTTGAACGAGGACCATTG
GATGGGAAGAGAAAACTCAGGAGGTGGAGTCGAAGGGTTAGGATTGCAAAGACTTGGATACAT
TCTAGAAGAAATGAATCGGGCACCAGGAGGAAAGATGTACGCAGATGACACTGCTGGCTGGGA
CACCCGCATTAGTAAGTTTGATCTGGAGAATGAAGCTCTGATTACCAACCAAATGGAGGAAGG
GCACAGAACTCTGGCGTTGGCCGTGATTAAATACACATACCAAAACAAAGTGGTGAAGGTTCTC
AGACCAGCTGAAGGAGGAAAAACAGTTATGGACATCATTTCAAGACAAGACCAGAGAGGGAG
TGGACAAGTTGTCACTTATGCTCTCAACACATTCACCAACTTGGTGGTGCAGCTTATCCGGAAC
ATGGAAGCTGAGGAAGTGTTAGAGATGCAAGACTTATGGTTGTTGAGGAAGCCAGAGAAAGTG
ACCAGATGGTTGCAGAGCAATGGATGGGATAGACTCAAACGAATGGCGGTCAGTGGAGATGAC
TGCGTTGTGAAGCCAATCGATGATAGGTTTGCACATGCCCTCAGGTTCTTGAATGACATGGGAA
AAGTTAGGAAAGACACACAGGAGTGGAAACCCTCGACTGGATGGAGCAATTGGGAAGAAGTCC
CGTTCTGCTCCCACCACTTCAACAAGCTGTACCTCAAGGATGGGAGATCCATTGTGGTCCCTTGC
CGCCACCAAGATGAACTGATTGGCCGAGCTCGCGTCTCACCAGGGGCAGGATGGAGCATCCGG
GAGACTGCCTGTCTTGCAAAATCATATGCGCAGATGTGGCAGCTCCTTTATTTCCACAGAAGAG
ACCTTCGACTGATGGCTAATGCCATTTGCTCGGCTGTGCCAGTTGACTGGGTACCAACTGGGAG
AACCACCTGGTCAATCCATGGAAAGGGAGAATGGATGACCACTGAGGACATGCTCATGGTGTG
GAATAGAGTGTGGATTGAGGAGAACGACCATATGGAGGACAAGACTCCTGTAACAAAATGGAC
AGACATTCCCTATCTAGGAAAAAGGGAGGACTTATGGTGTGGATCCCTTATAGGGCACAGACCC
CGCACCACTTGGGCTGAAAACATCAAAGACACAGTCAACATGGTGCGCAGGATCATAGGTGAT
GAAGAAAAGTACATGGACTATCTATCCACCCAAGTCCGCTACTTGGGTGAGGAAGGGTCCACA
CCCGGAGTGTTGTAAGCACCAATTTTAGTGTTGTCAGGCCTGCTAGTCAGCCACAGTTTGGGGA
AAGCTGTGCAGCCTGTAACCCCCCCAGGAGAAGCTGGGAAACCAAGCTCATAGTCAGGCCGAG
AACGCCATGGCACGGAAGAAGCCATGCTGCCTGTGAGCCCCTCAGAGGACACTGAGTCAAAAA
ACCCCACGCGCTTGGAAGCGCAGGATGGGAAAAGAAGGTGGCGACCTTCCCCACCCTTCAATC
TGGGGCCTGAACTGGAGACTAGCTGTGAATCTCCAGCAGAGGGACTAGTGGTTAGAGGAGACC
CCCCGGAAAACGCAAAACAGCATATTGACGTGGGAAAGACCAGAGACTCCATGAGTTTCCACC
ACGCTGGCCGCCAGGCACAGATCGCCGAACTTCGGCGGCCGGTGTGGGGAAATCCATGGTTTCT
SEQ ID NO 2: Zika virus envelope gene
ATCAGGTGCATTGGAGTCAGCAATAGAGACTTCGTGGAGGGCATGTCAGGTGGGACCTGGGTT
GATGTTGTCTTGGAACATGGAGGCTGCGTTACCGTGATGGCACAGGACAAGCCAACAGTCGAC
ATAGAGTTGGTCACGACGACGGTTAGTAACATGGCCGAGGTAAGATCCTATTGCTACGAGGCAT
CGATATCGGACATGGCTTCGGACAGTCGTTGCCCAACACAAGGTGAAGCCTACCTTGACAAGCA
ATCAGACACTCAATATGTCTGCAAAAGAACATTAGTGGACAGAGGTTGGGGAAACGGTTGTGG
ACTTTTTGGCAAAGGGAGCTTGGTGACATGTGCCAAGTTTACGTGTTCTAAGAAGATGACCGGG
AAGAGCATTCAACCGGAAAATCTGGAGTATCGGATAATGCTATCAGTGCATGGCTCCCAGCATA
GCGGGATGATTGGATATGAAACTGACGAAGATAGAGCGAAAGTCGAGGTTACGCCTAATTCAC
CAAGAGCGGAAGCAACCTTGGGAGGCTTTGGAAGCTTAGGACTTGACTGTGAACCAAGGACAG
GCCTTGACTTTTCAGATCTGTATTACCTGACCATGAACAATAAGCATTGGTTGGTGCACAAAGA
GTGGTTTCATGACATCCCATTGCCTTGGCATGCTGGGGCAGACACCGGAACTCCACACTGGAAC
AACAAAGAGGCATTGGTAGAATTCAAGGATGCCCACGCCAAGAGGCAAACCGTCGTCGTTCTG
GGGAGCCAGGAAGGAGCCGTTCACACGGCTCTCGCTGGAGCTCTAGAGGCTGAGATGGATGGT
GCAAAGGGAAGGCTGTTCTCTGGCCATTTGAAATGCCGCCTAAAAATGGACAAGCTTAGATTGA
AGGGCGTGTCATATTCCTTGTGCACTGCGGCATTCACATTCACCAAGGTCCCAGCTGAAACACT
GCATGGAACAGTCACAGTGGAGGTGCAGTATGCAGGGACAGATGGACCCTGCAAGATCCCAGT
CCAGATGGCGGTGGACATGCAGACCCTGACCCCAGTTGGAAGGCTGATAACCGCCAACCCCGT
GATTACTGAAAGCACTGAGAACTCAAAGATGATGTTGGAGCTTGACCCACCATTTGGGGATTCT
TACATTGTCATAGGAGTTGGGGACAAGAAAATCACCCACCACTGGCATAGGAGTGGTAGCACC
ATCGGAAAGGCATTTGAGGCCACTGTGAGAGGCGCCAAGAGAATGGCAGTCCTGGGGGATACA
GCCTGGGACTTCGGATCAGTCGGGGGTGTGTTCAACTCACTGGGTAAGGGCATTCACCAGATTT
TTGGAGCAGCCTTCAAATCACTGTTTGGAGGAATGTCCTGGTTCTCACAGATCCTCATAGGCAC
GCTGCTAGTGTGGTTAGGTTTGAACACAAAGAATGGATCTATCTCCCTCACATGCTTGGCCCTG
GGGGGAGTGATGATCTTCCTCTCCACGGCTGTTTCTGCT
SEQ ID NO 3: Zika virus non-structural genes 1-5
AGTTGTTGATCTGTGTGAGTCAGACTGCGACAGTTCGAGTCTGAAGCGAGAGCTAACAACAGTA
TCAACAGGTTTAATTTGGATTTGGAAACGAGAGTTTCTGGTCATGAAAAACCCCAAAGAAGAA
ATCCGGAGGATCCGGATTGTCAATATGCTAAAACGCGGAGTAGCCCGTGTAAACCCCTTGGGA
GGTTTGAAGAGGTTGCCAGCCGGACTTCTGCTGGGTCATGGACCCATCAGAATGGTTTTGGCGA
TACTAGCCTTTTTGAGATTTACAGCAATCAAGCCATCACTGGGCCTTATCAACAGATGGGGTTC
CGTGGGGAAAAAAGAGGCTATGGAAATAATAAAGAAGTTCAAGAAAGATCTTGCTGCCATGTT
GAGAATAATCAATGCTAGGAAAGAGAGGAAGAGACGTGGCGCAGACACCAGCATCGGAATCA
TTGGCCTCCTGCTGACTACAGCCATGGCAGCAGAGATCACTAGACGCGGGAGTGCATACTACAT
GTACTTGGATAGGAGCGATGCCGGGAAGGCCATTTCGTTTGCTACCACATTGGGAGTGAACAAG
TGCCACGTACAGATCATGGACCTCGGGCACATGTGTGACGCCACCATGAGTTATGAGTGCCCTA
TGCTGGATGAGGGAGTGGAACCAGATGATGTCGATTGCTGGTGCAACACGACATCAACTTGGG
TTGTGTACGGAACCTGTCATCACAAAAAAGGTGAGGCACGGCGATCTAGAAGAGCCGTGACGC
TCCCTTCTCACTCTACAAGGAAGTTGCAAACGCGGTCGCAGACCTGGTTAGAATCAAGAGAATA
CACGAAGCACTTGATCAAGGTTGAAAACTGGATATTCAGGAACCCCGGGTTTGCGCTAGTGGCC
GTTGCCATTGCCTGGCTTTTGGGAAGCTCGACGAGCCAAAAAGTCATATACTTGGTCATGATAC
TGCTGATTGCCCCGGCATACAGTATCAGGTGCATTGGAGTCAGCAATAGAGACTTCGTGGAGGG
CATGTCAGGTGGGACCTGGGTTGATGTTGTCTTGGAACATGGAGGCTGCGTTACCGTGATGGCA
CAGGACAAGCCAACAGTCGACATAGAGTTGGTCACGACGACGGTTAGTAACATGGCCGAGGTA
AGATCCTATTGCTACGAGGCATCGATATCGGACATGGCTTCGGACAGTCGTTGCCCAACACAAG
GTGAAGCCTACCTTGACAAGCAATCAGACACTCAATATGTCTGCAAAAGAACATTAGTGGACA
GAGGTTGGGGAAACGGTTGTGGACTTTTTGGCAAAGGGAGCTTGGTGACATGTGCCAAGTTTAC
GTGTTCTAAGAAGATGACCGGGAAGAGCATTCAACCGGAAAATCTGGAGTATCGGATAATGCT
ATCAGTGCATGGCTCCCAGCATAGCGGGATGATTGGATATGAAACTGACGAAGATAGAGCGAA
AGTCGAGGTTACGCCTAATTCACCAAGAGCGGAAGCAACCTTGGGAGGCTTTGGAAGCTTAGG
ACTTGACTGTGAACCAAGGACAGGCCTTGACTTTTCAGATCTGTATTACCTGACCATGAACAAT
AAGCATTGGTTGGTGCACAAAGAGTGGTTTCATGACATCCCATTGCCTTGGCATGCTGGGGCAG
ACACCGGAACTCCACACTGGAACAACAAAGAGGCATTGGTAGAATTCAAGGATGCCCACGCCA
AGAGGCAAACCGTCGTCGTTCTGGGGAGCCAGGAAGGAGCCGTTCACACGGCTCTCGCTGGAG
CTCTAGAGGCTGAGATGGATGGTGCAAAGGGAAGGCTGTTCTCTGGCCATTTGAAATGCCGCCT
AAAAATGGACAAGCTTAGATTGAAGGGCGTGTCATATTCCTTGTGCACTGCGGCATTCACATTC
ACCAAGGTCCCAGCTGAAACACTGCATGGAACAGTCACAGTGGAGGTGCAGTATGCAGGGACA
GATGGACCCTGCAAGATCCCAGTCCAGATGGCGGTGGACATGCAGACCCTGACCCCAGTTGGA
AGGCTGATAACCGCCAACCCCGTGATTACTGAAAGCACTGAGAACTCAAAGATGATGTTGGAG
CTTGACCCACCATTTGGGGATTCTTACATTGTCATAGGAGTTGGGGACAAGAAAATCACCCACC
ACTGGCATAGGAGTGGTAGCACCATCGGAAAGGCATTTGAGGCCACTGTGAGAGGCGCCAAGA
GAATGGCAGTCCTGGGGGATACAGCCTGGGACTTCGGATCAGTCGGGGGTGTGTTCAACTCACT
GGGTAAGGGCATTCACCAGATTTTTGGAGCAGCCTTCAAATCACTGTTTGGAGGAATGTCCTGG
TTCTCACAGATCCTCATAGGCACGCTGCTAGTGTGGTTAGGTTTGAACACAAAGAATGGATCTA
TCTCCCTCACATGCTTGGCCCTGGGGGGAGTGATGATCTTCCTCTCCACGGCTGTTTCTGCTGAC
GTGGGGTGCTCAGTGGACTTCTCAAAAAAGGAAACGAGATGTGGCACGGGGGTATTCATCTAT
AATGATGTTGAAGCCTGGAGGGACCGGTACAAGTACCATCCTGACTCCCCCCGCAGATTGGCAG
CAGCAGTCAAGCAGGCCTGGGAAGAGGGGATCTGTGGGATCTCATCCGTTTCAAGAATGGAAA
ACATCATGTGGAAATCAGTAGAAGGGGAGCTCAATGCTATCCTAGAGGAGAATGGAGTTCAAC
TGACAGTTGTTGTGGGATCTGTAAAAAACCCCATGTGGAGAGGTCCACAAAGATTGCCAGTGCC
TGTGAATGAGCTGCCCCATGGCTGGAAAGCCTGGGGGAAATCGTATTTTGTTAGGGCGGCAAA
GACCAACAACAGTTTTGTTGTCGACGGTGACACACTGAAGGAATGTCCGCTTGAGCACAGAGC
ATGGAATAGTTTTCTTGTGGAGGATCACGGGTTTGGAGTCTTCCACACCAGTGTCTGGCTTAAG
GTCAGAGAAGATTACTCATTAGAATGTGACCCAGCCGTCATAGGAACAGCTGTTAAGGGAAGG
GAGGCCGCGCACAGTGATCTGGGCTATTGGATTGAAAGTGAAAAGAATGACACATGGAGGCTG
AAGAGGGCCCACCTGATTGAGATGAAAACATGTGAATGGCCAAAGTCTCACACATTGTGGACA
GATGGAGTAGAAGAAAGTGATCTTATCATACCCAAGTCTTTAGCTGGTCCACTCAGCCACCACA
ACACCAGAGAGGGTTACAGAACCCAAGTGAAAGGGCCATGGCACAGTGAAGAGCTTGAAATCC
GGTTTGAGGAATGTCCAGGCACCAAGGTTTACGTGGAGGAGACATGCGGAACTAGAGGACCAT
CTCTGAGATCAACTACTGCAAGTGGAAGGGTCATTGAGGAATGGTGCTGTAGGGAATGCACAA
TGCCCCCACTATCGTTTCGAGCAAAAGACGGCTGCTGGTATGGAATGGAGATAAGGCCCAGGA
AAGAACCAGAGAGCAACTTAGTGAGGTCAATGGTGACAGCGGGGTCAACCGATCATATGGACC
ACTTCTCTCTTGGAGTGCTTGTGATTCTACTCATGGTGCAGGAGGGGTTGAAGAAGAGAATGAC
CACAAAGATCATCATGAGCACATCAATGGCAGTGCTGGTAGTCATGATCTTGGGAGGATTTTCA
ATGAGTGACCTGGCCAAGCTTGTGATCCTGATGGGTGCTACTTTCGCAGAAATGAACACTGGAG
GAGATGTAGCTCACTTGGCATTGGTAGCGGCATTTAAAGTCAGACCAGCCTTGCTGGTCTCCTT
CATTTTCAGAGCCAATTGGACACCCCGTGAGAGCATGCTGCTAGCCCTGGCTTCGTGTCTTCTGC
AAACTGCGATCTCTGCTCTTGAAGGTGACTTGATGGTCCTCATTAATGGATTTGCTTTGGCCTGG
TTGGCAATTCGAGCAATGGCCGTGCCACGCACTGACAACATCGCTCTACCAATCTTGGCTGCTC
TAACACCACTAGCTCGAGGCACACTGCTCGTGGCATGGAGAGCGGGCCTGGCTACTTGTGGAG
GGATCATGCTCCTCTCCCTGAAAGGGAAAGGTAGTGTGAAGAAGAACCTGCCATTTGTCATGGC
CCTGGGATTGACAGCTGTGAGGGTAGTAGACCCTATTAATGTGGTAGGACTACTGTTACTCACA
AGGAGTGGGAAGCGGAGCTGGCCCCCTAGTGAAGTTCTCACAGCCGTTGGCCTGATATGTGCAC
TGGCCGGAGGGTTTGCCAAGGCAGACATTGAGATGGCTGGACCCATGGCTGCAGTAGGCTTGCT
AATTGTCAGCTATGTGGTCTCGGGAAAGAGTGTGGACATGTACATTGAAAGAGCAGGTGACAT
CACATGGGAAAAGGACGCGGAAGTCACTGGAAACAGTCCTCGGCTTGACGTGGCACTGGATGA
GAGTGGTGACTTCTCCTTGGTAGAGGAAGATGGTCCACCCATGAGAGAGATCATACTCAAGGTG
GTCCTGATGGCCATCTGTGGCATGAACCCAATAGCTATACCTTTTGCTGCAGGAGCGTGGTATG
TGTATGTGAAGACTGGGAAAAGGAGTGGCGCCCTCTGGGACGTGCCTGCTCCCAAAGAAGTGA
AGAAAGGAGAGACCACAGATGGAGTGTACAGAGTGATGACTCGCAGACTGCTAGGTTCAACAC
AGGTTGGAGTGGGAGTCATGCAAGAGGGAGTCTTCCACACCATGTGGCACGTTACAAAAGGAG
CCGCACTGAGGAGCGGTGAGGGAAGACTTGATCCATACTGGGGGGATGTCAAGCAGGACTTGG
TGTCATACTGTGGGCCTTGGAAGTTGGATGCAGCTTGGGATGGACTCAGCGAGGTACAGCTTTT
GGCCGTACCTCCCGGAGAGAGGGCCAGAAACATTCAGACCCTGCCTGGAATATTCAAGACAAA
GGACGGGGACATCGGAGCAGTTGCTCTGGACTACCCTGCAGGGACCTCAGGATCTCCGATCCTA
GACAAATGTGGAAGAGTGATAGGACTCTATGGCAATGGGGTTGTGATCAAGAATGGAAGCTAT
GTTAGTGCTATAACCCAGGGAAAGAGGGAGGAGGAGACTCCGGTTGAATGTTTCGAACCCTCG
ATGCTGAAGAAGAAGCAGCTAACTGTCTTGGATCTGCATCCAGGAGCCGGAAAAACCAGGAGA
GTTCTTCCTGAAATAGTCCGTGAAGCCATAAAAAAGAGACTCCGGACAGTGATCTTGGCACCAA
CTAGGGTTGTCGCTGCTGAGATGGAGGAGGCCTTGAGAGGACTTCCGGTGCGTTACATGACAAC
AGCAGTCAACGTCACCCATTCTGGGACAGAAATCGTTGATTTGATGTGCCATGCCACTTTCACTT
CACGCTTACTACAACCCATCAGAGTCCCTAATTACAATCTCAACATCATGGATGAAGCCCACTT
CACAGACCCCTCAAGTATAGCTGCAAGAGGATACATATCAACAAGGGTTGAAATGGGCGAGGC
GGCTGCCATTTTTATGACTGCCACACCACCAGGAACCCGTGATGCGTTTCCTGACTCTAACTCAC
CAATCATGGACACAGAAGTGGAAGTCCCAGAGAGAGCCTGGAGCTCAGGCTTTGATTGGGTGA
CAGACCATTCTGGGAAAACAGTTTGGTTCGTTCCAAGCGTGAGAAACGGAAATGAAATCGCAG
CCTGTCTGACAAAGGCTGGAAAGCGGGTCATACAGCTCAGCAGGAAGACTTTTGAGACAGAAT
TTCAGAAAACAAAAAATCAAGAGTGGGACTTTGTCATAACAACTGACATCTCAGAGATGGGCG
CCAACTTCAAGGCTGACCGGGTCATAGACTCTAGGAGATGCCTAAAACCAGTCATACTTGATGG
TGAGAGAGTCATCTTGGCTGGGCCCATGCCTGTCACGCATGCTAGTGCTGCTCAGAGGAGAGGA
CGTATAGGCAGGAACCCTAACAAACCTGGAGATGAGTACATGTATGGAGGTGGGTGTGCAGAG
ACTGATGAAGGCCATGCACACTGGCTTGAAGCAAGAATGCTTCTTGACAACATCTACCTCCAGG
ATGGCCTCATAGCCTCGCTCTATCGGCCTGAGGCCGATAAGGTAGCCGCCATTGAGGGAGAGTT
TAAGCTGAGGACAGAGCAAAGGAAGACCTTCGTGGAACTCATGAAGAGAGGAGACCTTCCCGT
CTGGCTAGCCTATCAGGTTGCATCTGCCGGAATAACTTACACAGACAGAAGATGGTGCTTTGAT
GGCACAACCAACAACACCATAATGGAAGACAGTGTACCAGCAGAGGTTTGGACAAAGTATGGA
GAGAAGAGAGTGCTCAAACCGAGATGGATGGATGCTAGGGTCTGTTCAGACCATGCGGCCCTG
AAGTCGTTCAAAGAATTCGCCGCTGGAAAAAGAGGAGCGGCTTTGGGAGTAATGGAGGCCCTG
GGAACACTGCCAGGACACATGACAGAGAGGTTTCAGGAAGCCATTGACAACCTCGCCGTGCTC
ATGCGAGCAGAGACTGGAAGCAGGCCTTATAAGGCAGCGGCAGCCCAACTGCCGGAGACCCTA
GAGACCATTATGCTCTTAGGTTTGCTGGGAACAGTTTCACTGGGGATCTTCTTCGTCTTGATGCG
GAATAAGGGCATCGGGAAGATGGGCTTTGGAATGGTAACCCTTGGGGCCAGTGCATGGCTCAT
GTGGCTTTCGGAAATTGAACCAGCCAGAATTGCATGTGTCCTCATTGTTGTGTTTTTATTACTGG
TGGTGCTCATACCCGAGCCAGAGAAGCAAAGATCTCCCCAAGATAACCAGATGGCAATTATCA
TCATGGTGGCAGTGGGCCTTCTAGGTTTGATAACTGCAAACGAACTTGGATGGCTGGAAAGAAC
AAAAAATGACATAGCTCATCTAATGGGAAGGAGAGAAGAAGGAGCAACCATGGGATTCTCAAT
GGACATTGATCTGCGGCCAGCCTCCGCCTGGGCTATCTATGCCGCATTGACAACTCTCATCACC
CCAGCTGTCCAACATGCGGTAACCACTTCATACAACAACTACTCCTTAATGGCGATGGCCACAC
AAGCTGGAGTGCTGTTTGGCATGGGCAAAGGGATGCCATTTATGCATGGGGACCTTGGAGTCCC
GCTGCTAATGATGGGTTGCTATTCACAATTAACACCCCTGACTCTGATAGTAGCTATCATTCTGC
TTGTGGCGCACTACATGTACTTGATCCCAGGCCTACAAGCGGCAGCAGCGCGTGCTGCCCAGAA
AAGGACAGCAGCTGGCATCATGAAGAATCCCGTTGTGGATGGAATAGTGGTAACTGACATTGA
CACAATGACAATAGACCCCCAGGTGGAGAAGAAGATGGGACAAGTGTTACTCATAGCAGTAGC
CATCTCCAGTGCTGTGCTGCTGCGGACCGCCTGGGGATGGGGGGAGGCTGGAGCTCTGATCACA
GCAGCGACCTCCACCTTGTGGGAAGGCTCTCCAAACAAATACTGGAACTCCTCTACAGCCACCT
CACTGTGCAACATCTTCAGAGGAAGCTATCTGGCAGGAGCTTCCCTTATCTATACAGTGACGAG
AAACGCTGGCCTGGTTAAGAGACGTGGAGGTGGGACGGGAGAGACTCTGGGAGAGAAGTGGA
AAGCTCGTCTGAATCAGATGTCGGCCCTGGAGTTCTACTCTTATAAAAAGTCAGGTATCACTGA
AGTGTGTAGAGAGGAGGCTCGCCGTGCCCTCAAGGATGGAGTGGCCACAGGAGGACATGCCGT
ATCCCGGGGAAGTGCAAAGATCAGATGGTTGGAGGAGAGAGGATATCTGCAGCCCTATGGGAA
GGTTGTTGACCTCGGATGTGGCAGAGGGGGCTGGAGCTATTATGCCGCCACCATCCGCAAAGTG
CAGGAGGTGAGAGGATACACAAAGGGAGGTCCCGGTCATGAAGAACCCATGCTGGTGCAAAGC
TATGGGTGGAACATAGTTCGTCTCAAGAGTGGAGTGGACGTCTTCCACATGGCGGCTGAGCCGT
GTGACACTCTGCTGTGTGACATAGGTGAGTCATCATCTAGTCCTGAAGTGGAAGAGACACGAAC
ACTCAGAGTGCTCTCTATGGTGGGGGACTGGCTTGAAAAAAGACCAGGGGCCTTCTGTATAAAG
GTGCTGTGCCCATACACCAGCACTATGATGGAAACCATGGAGCGACTGCAACGTAGGCATGGG
GGAGGATTAGTCAGAGTGCCATTGTGTCGCAACTCCACACATGAGATGTACTGGGTCTCTGGGG
CAAAGAGCAACATCATAAAAAGTGTGTCCACCACAAGTCAGCTCCTCCTGGGACGCATGGATG
GCCCCAGGAGGCCAGTGAAATATGAGGAGGATGTGAACCTCGGCTCGGGTACACGAGCTGTGG
CAAGCTGTGCTGAGGCTCCTAACATGAAAATCATCGGCAGGCGCATTGAGAGAATCCGCAATG
AACATGCAGAAACATGGTTTCTTGATGAAAACCACCCATACAGGACATGGGCCTACCATGGGA
GCTACGAAGCCCCCACGCAAGGATCAGCGTCTTCCCTCGTGAACGGGGTTGTTAGACTCCTGTC
AAAGCCTTGGGACGTGGTGACTGGAGTTACAGGAATAGCCATGACTGACACCACACCATACGG
CCAACAAAGAGTCTTCAAAGAAAAAGTGGACACCAGGGTGCCAGATCCCCAAGAAGGCACTCG
CCAGGTAATGAACATAGTCTCTTCCTGGCTGTGGAAGGAGCTGGGGAAACGCAAGCGGCCACG
CGTCTGCACCAAAGAAGAGTTTATCAACAAGGTGCGCAGCAATGCAGCACTGGGAGCAATATT
TGAAGAGGAAAAAGAATGGAAGACGGCTGTGGAAGCTGTGAATGATCCAAGGTTTTGGGCCCT
AGTGGATAGGGAGAGAGAACACCACCTGAGAGGAGAGTGTCACAGCTGTGTGTACAACATGAT
GGGAAAAAGAGAAAAGAAGCAAGGAGAGTTCGGGAAAGCAAAAGGTAGCCGCGCCATCTGGT
ACATGTGGTTGGGAGCCAGATTCTTGGAGTTTGAAGCCCTTGGATTCTTGAACGAGGACCATTG
GATGGGAAGAGAAAACTCAGGAGGTGGAGTCGAAGGGTTAGGATTGCAAAGACTTGGATACAT
TCTAGAAGAAATGAATCGGGCACCAGGAGGAAAGATGTACGCAGATGACACTGCTGGCTGGGA
CACCCGCATTAGTAAGTTTGATCTGGAGAATGAAGCTCTGATTACCAACCAAATGGAGGAAGG
GCACAGAACTCTGGCGTTGGCCGTGATTAAATACACATACCAAAACAAAGTGGTGAAGGTTCTC
AGACCAGCTGAAGGAGGAAAAACAGTTATGGACATCATTTCAAGACAAGACCAGAGAGGGAG
TGGACAAGTTGTCACTTATGCTCTCAACACATTCACCAACTTGGTGGTGCAGCTTATCCGGAAC
ATGGAAGCTGAGGAAGTGTTAGAGATGCAAGACTTATGGTTGTTGAGGAAGCCAGAGAAAGTG
ACCAGATGGTTGCAGAGCAATGGATGGGATAGACTCAAACGAATGGCGGTCAGTGGAGATGAC
TGCGTTGTGAAGCCAATCGATGATAGGTTTGCACATGCCCTCAGGTTCTTGAATGACATGGGAA
AAGTTAGGAAAGACACACAGGAGTGGAAACCCTCGACTGGATGGAGCAATTGGGAAGAAGTCC
CGTTCTGCTCCCACCACTTCAACAAGCTGTACCTCAAGGATGGGAGATCCATTGTGGTCCCTTGC
CGCCACCAAGATGAACTGATTGGCCGAGCTCGCGTCTCACCAGGGGCAGGATGGAGCATCCGG
GAGACTGCCTGTCTTGCAAAATCATATGCGCAGATGTGGCAGCTCCTTTATTTCCACAGAAGAG
ACCTTCGACTGATGGCTAATGCCATTTGCTCGGCTGTGCCAGTTGACTGGGTACCAACTGGGAG
AACCACCTGGTCAATCCATGGAAAGGGAGAATGGATGACCACTGAGGACATGCTCATGGTGTG
GAATAGAGTGTGGATTGAGGAGAACGACCATATGGAGGACAAGACTCCTGTAACAAAATGGAC
AGACATTCCCTATCTAGGAAAAAGGGAGGACTTATGGTGTGGATCCCTTATAGGGCACAGACCC
CGCACCACTTGGGCTGAAAACATCAAAGACACAGTCAACATGGTGCGCAGGATCATAGGTGAT
GAAGAAAAGTACATGGACTATCTATCCACCCAAGTCCGCTACTTGGGTGAGGAAGGGTCCACA
CCCGGAGTGTTG
SEQ ID NO 4: Zika virus non-structural gene 3
AGTGGCGCCCTCTGGGACGTGCCTGCTCCCAAAGAAGTGAAGAAAGGAGAGACCACAGATGGA
GTGTACAGAGTGATGACTCGCAGACTGCTAGGTTCAACACAGGTTGGAGTGGGAGTCATGCAA
GAGGGAGTCTTCCACACCATGTGGCACGTTACAAAAGGAGCCGCACTGAGGAGCGGTGAGGGA
AGACTTGATCCATACTGGGGGGATGTCAAGCAGGACTTGGTGTCATACTGTGGGCCTTGGAAGT
TGGATGCAGCTTGGGATGGACTCAGCGAGGTACAGCTTTTGGCCGTACCTCCCGGAGAGAGGG
CCAGAAACATTCAGACCCTGCCTGGAATATTCAAGACAAAGGACGGGGACATCGGAGCAGTTG
CTCTGGACTACCCTGCAGGGACCTCAGGATCTCCGATCCTAGACAAATGTGGAAGAGTGATAGG
ACTCTATGGCAATGGGGTTGTGATCAAGAATGGAAGCTATGTTAGTGCTATAACCCAGGGAAA
GAGGGAGGAGGAGACTCCGGTTGAATGTTTCGAACCCTCGATGCTGAAGAAGAAGCAGCTAAC
TGTCTTGGATCTGCATCCAGGAGCCGGAAAAACCAGGAGAGTTCTTCCTGAAATAGTCCGTGAA
GCCATAAAAAAGAGACTCCGGACAGTGATCTTGGCACCAACTAGGGTTGTCGCTGCTGAGATG
GAGGAGGCCTTGAGAGGACTTCCGGTGCGTTACATGACAACAGCAGTCAACGTCACCCATTCTG
GGACAGAAATCGTTGATTTGATGTGCCATGCCACTTTCACTTCACGCTTACTACAACCCATCAG
AGTCCCTAATTACAATCTCAACATCATGGATGAAGCCCACTTCACAGACCCCTCAAGTATAGCT
GCAAGAGGATACATATCAACAAGGGTTGAAATGGGCGAGGCGGCTGCCATTTTTATGACTGCC
ACACCACCAGGAACCCGTGATGCGTTTCCTGACTCTAACTCACCAATCATGGACACAGAAGTGG
AAGTCCCAGAGAGAGCCTGGAGCTCAGGCTTTGATTGGGTGACAGACCATTCTGGGAAAACAG
TTTGGTTCGTTCCAAGCGTGAGAAACGGAAATGAAATCGCAGCCTGTCTGACAAAGGCTGGAA
AGCGGGTCATACAGCTCAGCAGGAAGACTTTTGAGACAGAATTTCAGAAAACAAAAAATCAAG
AGTGGGACTTTGTCATAACAACTGACATCTCAGAGATGGGCGCCAACTTCAAGGCTGACCGGGT
CATAGACTCTAGGAGATGCCTAAAACCAGTCATACTTGATGGTGAGAGAGTCATCTTGGCTGGG
CCCATGCCTGTCACGCATGCTAGTGCTGCTCAGAGGAGAGGACGTATAGGCAGGAACCCTAAC
AAACCTGGAGATGAGTACATGTATGGAGGTGGGTGTGCAGAGACTGATGAAGGCCATGCACAC
TGGCTTGAAGCAAGAATGCTTCTTGACAACATCTACCTCCAGGATGGCCTCATAGCCTCGCTCT
ATCGGCCTGAGGCCGATAAGGTAGCCGCCATTGAGGGAGAGTTTAAGCTGAGGACAGAGCAAA
GGAAGACCTTCGTGGAACTCATGAAGAGAGGAGACCTTCCCGTCTGGCTAGCCTATCAGGTTGC
ATCTGCCGGAATAACTTACACAGACAGAAGATGGTGCTTTGATGGCACAACCAACAACACCAT
AATGGAAGACAGTGTACCAGCAGAGGTTTGGACAAAGTATGGAGAGAAGAGAGTGCTCAAACC
GAGATGGATGGATGCTAGGGTCTGTTCAGACCATGCGGCCCTGAAGTCGTTCAAAGAATTCGCC
GCTGGAAAAAGA
SEQ ID NO 5: Zika virus non-structural gene 5
GGAGGTGGGACGGGAGAGACTCTGGGAGAGAAGTGGAAAGCTCGTCTGAATCAGATGTCGGCC
CTGGAGTTCTACTCTTATAAAAAGTCAGGTATCACTGAAGTGTGTAGAGAGGAGGCTCGCCGTG
CCCTCAAGGATGGAGTGGCCACAGGAGGACATGCCGTATCCCGGGGAAGTGCAAAGATCAGAT
GGTTGGAGGAGAGAGGATATCTGCAGCCCTATGGGAAGGTTGTTGACCTCGGATGTGGCAGAG
GGGGCTGGAGCTATTATGCCGCCACCATCCGCAAAGTGCAGGAGGTGAGAGGATACACAAAGG
GAGGTCCCGGTCATGAAGAACCCATGCTGGTGCAAAGCTATGGGTGGAACATAGTTCGTCTCAA
GAGTGGAGTGGACGTCTTCCACATGGCGGCTGAGCCGTGTGACACTCTGCTGTGTGACATAGGT
GAGTCATCATCTAGTCCTGAAGTGGAAGAGACACGAACACTCAGAGTGCTCTCTATGGTGGGG
GACTGGCTTGAAAAAAGACCAGGGGCCTTCTGTATAAAGGTGCTGTGCCCATACACCAGCACTA
TGATGGAAACCATGGAGCGACTGCAACGTAGGCATGGGGGAGGATTAGTCAGAGTGCCATTGT
GTCGCAACTCCACACATGAGATGTACTGGGTCTCTGGGGCAAAGAGCAACATCATAAAAAGTG
TGTCCACCACAAGTCAGCTCCTCCTGGGACGCATGGATGGCCCCAGGAGGCCAGTGAAATATG
AGGAGGATGTGAACCTCGGCTCGGGTACACGAGCTGTGGCAAGCTGTGCTGAGGCTCCTAACA
TGAAAATCATCGGCAGGCGCATTGAGAGAATCCGCAATGAACATGCAGAAACATGGTTTCTTG
ATGAAAACCACCCATACAGGACATGGGCCTACCATGGGAGCTACGAAGCCCCCACGCAAGGAT
CAGCGTCTTCCCTCGTGAACGGGGTTGTTAGACTCCTGTCAAAGCCTTGGGACGTGGTGACTGG
AGTTACAGGAATAGCCATGACTGACACCACACCATACGGCCAACAAAGAGTCTTCAAAGAAAA
AGTGGACACCAGGGTGCCAGATCCCCAAGAAGGCACTCGCCAGGTAATGAACATAGTCTCTTC
CTGGCTGTGGAAGGAGCTGGGGAAACGCAAGCGGCCACGCGTCTGCACCAAAGAAGAGTTTAT
CAACAAGGTGCGCAGCAATGCAGCACTGGGAGCAATATTTGAAGAGGAAAAAGAATGGAAGA
CGGCTGTGGAAGCTGTGAATGATCCAAGGTTTTGGGCCCTAGTGGATAGGGAGAGAGAACACC
ACCTGAGAGGAGAGTGTCACAGCTGTGTGTACAACATGATGGGAAAAAGAGAAAAGAAGCAA
GGAGAGTTCGGGAAAGCAAAAGGTAGCCGCGCCATCTGGTACATGTGGTTGGGAGCCAGATTC
TTGGAGTTTGAAGCCCTTGGATTCTTGAACGAGGACCATTGGATGGGAAGAGAAAACTCAGGA
GGTGGAGTCGAAGGGTTAGGATTGCAAAGACTTGGATACATTCTAGAAGAAATGAATCGGGCA
CCAGGAGGAAAGATGTACGCAGATGACACTGCTGGCTGGGACACCCGCATTAGTAAGTTTGAT
CTGGAGAATGAAGCTCTGATTACCAACCAAATGGAGGAAGGGCACAGAACTCTGGCGTTGGCC
GTGATTAAATACACATACCAAAACAAAGTGGTGAAGGTTCTCAGACCAGCTGAAGGAGGAAAA
ACAGTTATGGACATCATTTCAAGACAAGACCAGAGAGGGAGTGGACAAGTTGTCACTTATGCTC
TCAACACATTCACCAACTTGGTGGTGCAGCTTATCCGGAACATGGAAGCTGAGGAAGTGTTAGA
GATGCAAGACTTATGGTTGTTGAGGAAGCCAGAGAAAGTGACCAGATGGTTGCAGAGCAATGG
ATGGGATAGACTCAAACGAATGGCGGTCAGTGGAGATGACTGCGTTGTGAAGCCAATCGATGA
TAGGTTTGCACATGCCCTCAGGTTCTTGAATGACATGGGAAAAGTTAGGAAAGACACACAGGA
GTGGAAACCCTCGACTGGATGGAGCAATTGGGAAGAAGTCCCGTTCTGCTCCCACCACTTCAAC
AAGCTGTACCTCAAGGATGGGAGATCCATTGTGGTCCCTTGCCGCCACCAAGATGAACTGATTG
GCCGAGCTCGCGTCTCACCAGGGGCAGGATGGAGCATCCGGGAGACTGCCTGTCTTGCAAAAT
CATATGCGCAGATGTGGCAGCTCCTTTATTTCCACAGAAGAGACCTTCGACTGATGGCTAATGC
CATTTGCTCGGCTGTGCCAGTTGACTGGGTACCAACTGGGAGAACCACCTGGTCAATCCATGGA
AAGGGAGAATGGATGACCACTGAGGACATGCTCATGGTGTGGAATAGAGTGTGGATTGAGGAG
AACGACCATATGGAGGACAAGACTCCTGTAACAAAATGGACAGACATTCCCTATCTAGGAAAA
AGGGAGGACTTATGGTGTGGATCCCTTATAGGGCACAGACCCCGCACCACTTGGGCTGAAAAC
ATCAAAGACACAGTCAACATGGTGCGCAGGATCATAGGTGATGAAGAAAAGTACATGGACTAT
CTATCCACCCAAGTCCGCTACTTGGGTGAGGAAGGGTCCACACCCGGAGTGTTG
SEQ ID NO 6 (ProbeR1Zika):
GGATGCTCCATCCTGCCCCTGG
SEQ ID NO 7 (ProbeR2Zika):
GGAGCTGCCACATCTGCGCATATG
SEQ ID NO 8 (ProbeR3Zika)
GAACCTGAGGGCATGTGCAAACC
SEQ ID NO 9 (ProbeZikaUniversalPolyA):
AAAAAAGCAAACCTATCATC
SEQ ID NO 10 (ProbeZikaUniversal):
GCAAACCTATCATC
SEQ ID NO 11 (ProbeZikaDegene1):
CTTCAGCTGGTCTGAG
SEQ ID NO 12 (ProbeZikaDegene2):
TTTCAGCTGGTCTAAG
SEQ ID NO 13 (Binding sequence for ProbeR1Zika)
CCAGGGGCAGGATGGAGCATCC
SEQ ID NO 14 (ProbeR2Zika)
CATATGCGCAGATGTGGCAGCTCC
SEQ ID NO 15 (ProbeR3Zika)
GGTTTGCACATGCCCTCAGGTTC
SEQ ID NO 16 (ProbeZikaUniversalPolyA and ProbeZikaUniversal)
GATGATAGGTTTGC
SEQ ID NO 17 (ProbeZikaDegene1 and ProbeZikaDegene2
(mismatch at positions 3 and 16))
CTCAGACCAGCTGAAG

1. A probe comprising a metal particle and at least one polynucleotide comprising a sequence substantially complementary to a region within the non-structural 5 (NS5) gene of a virus of the Flaviviridae family.

2. The probe of claim 1,
(a) wherein the virus is of the genus Flavivirus; or
(b) wherein the virus is Zika virus, West Nile virus, dengue virus, tick-borne encephalitis virus, yellow fever virus, Japanese encephalitis virus, cell fusing agent virus (CFAV), Palm Creek virus (PCV), or Parramatta River virus (PaRV); or
(c) wherein the virus is Zika virus.

3. The probe of claim 1, wherein the polynucleotide comprises:
(a) single-stranded DNA or single-stranded RNA; or
(b) a sequence substantially complementary to a region within the gene sequence encoding the RNA-dependent RNA polymerase protein or the 3′-untranslated region of the viral genome; or
(c) a sequence substantially complementary to a region within the non-structural 5 (NS5) gene wherein the sequence of the NS5 gene has at least 80% sequence identity to SEQ ID NO. 5; or
(d) a sequence substantially complementary to a region within the non-structural 5 (NS5) gene wherein the sequence of the NS5 gene has a sequence of SEQ ID NO. 5; or
(e) a sequence substantially complementary to a region within the viral genome that has the sequence between nucleotides 9182 to 9961 of SEQ ID NO 1; or
(f) a sequence substantially complementary to a region within the viral genome that has a sequence having at least 80% sequence identity to the sequence selected from SEQ ID NO 13, SEQ ID NO 14, SEQ ID NO 15, SEQ ID NO 16 or SEQ ID NO 17; or
(g) a sequence substantially complementary to a region within the viral genome that has the sequence selected from SEQ ID NO 13, SEQ ID NO 14, SEQ ID NO 15, SEQ ID NO 16 or SEQ ID NO 17; or
(h) a sequence with at least 80% sequence identity to one of SEQ ID NO 6, SEQ ID NO 7, SEQ ID NO 8, SEQ ID NO 9, SEQ ID NO 10, SEQ ID NO 11 or SEQ ID NO 12; or
(i) a sequence with at least 80% sequence identity to SEQ ID NO 10; or
(j) a sequence of one of SEQ ID NO 6, SEQ ID NO 7 , SEQ ID NO 8, SEQ ID NO 9, SEQ ID NO 10, SEQ ID NO 11 or SEQ ID NO 12; or
(k) a sequence of SEQ ID NO 10.

4. The probe of claim 1, wherein the polynucleotide:
(a) has a length of between 10 to 30 nucleotides; or
(b) further comprises a thiol group at the 3′ or 5′ terminus.

5. (canceled)

6. The probe of claim 1, wherein the metal particle:
(a) comprises gold, silver, a gold/silver alloy or an alloy of gold with another metal; or a combination of one or more of these metals; or
(b) comprises or consists substantially of gold; or
(c) is substantially spherical; or
(d) has a diameter between 10 nm to 20 nm.

7. The probe of claim 1, wherein:
(a) the at least one polynucleotide is linked to the particle through a thiol group at the 3′ or 5′ terminus of the polynucleotide; or
(b) the particle is conjugated to a plurality of polynucleotides; or
(c) the probe comprises between 100-200 copies of the polynucleotide; or
(d) the density of polynucleotides on the probe is between 20-40 pmol/cm²,

8. (canceled)

9. (canceled)

10. The probe according to claim 1, formulated as a composition.

11. (canceled)

12. A polynucleotide comprising a sequence substantially complementary to a region within the non-structural 5 (NS5) gene of Zika virus wherein the polynucleotide comprises a sequence of one of SEQ ID NO 6, SEQ ID NO 7, SEQ ID NO 8, SEQ ID NO 9, SEQ ID NO 10, SEQ ID NO 11 or SEQ ID NO 12.

13. (canceled)

14. (canceled)

15. The polynucleotide of claim 12, wherein:
(a) the polynucleotide has a length of between 10 to 30 nucleotides;
(b) the polynucleotide comprises SEQ ID NO 10: or
(c) the polynucleotide further comprises a thiol group at the 3′ or 5′ terminus;
optionally wherein the polynucleotide further comprises a linker between the polynucleotide and the thiol group at the 3′ or 5′ terminus
optionally wherein the linker comprises 5 to 10 nucleotides, optionally wherein the 5 to 10 nucleotides of the linker are at least 80% A and T nucleotides.

16. (canceled)

17. (canceled)

18. A composition or kit comprising the polynucleotide of claim 12;
optionally wherein the polynucleotide comprises a sequence that is bound or hybridised hybridized to a virus nucleic acid sequence, optionally a Zika virus nucleic acid sequence.

19. (canceled)

20. A kit comprising the probe of claim 1.

21. (canceled)

22. (canceled)

23. (canceled)

24. (Canceled)

25. (canceled)

26. A method for preparing the probe of claim 1, the method comprising:
(a) contacting a particle with a polynucleotide comprising a sequence substantially complementary to a region within the non-structural 5 (NS5) gene of a virus of the Flaviviridae family to bind the particle with the polynucleotide; and
(b) optionally contacting the particle and the polynucleotide with a salt source, to increase the salt concentration;
wherein the salt concentration preferably increases stepwise.

27. The method of claim 26, wherein a ratio of particle:
polynucleotide (w/w) is between 150:1 and 250:1.

28. The method of claim 26, wherein:
(a) the salt source is a concentrated salt solution; or
(b) the salt is an ionic halide; or
(c) increasing the salt concentration stepwise comprises increasing the salt concentration by up to between 0.1-0.2 M every at least 15 minutes.

29. A method of detecting a Flaviviridae; family virus or nucleic acid sequence thereof in a sample, the method comprising:
(a) contacting a probe of claim 1 with the sample;
(b) contacting the composition resulting from step (a) with a salt source; and
(c) optionally detecting either:
(i) a color change if the viral nucleic acid sequence is not present in the sample; or
(ii) no color change if the viral nucleic acid sequence is present in the sample,

30. The method of claim 29, wherein:
(a) the virus is Zika ⁻virus;
(b) the sample is a biological sample or a sample from a patient;
(c) the sample is used directly in the method;
(d) the sample is a liquid.

31. The method of claim 29, wherein:
(a) the probe is in solution or suspension, optionally at a concentration between 0.5 and 50 nM; or
(b) the probe is adhered or dried onto a solid support or matrix; or
(c) the contacting occurs over a period of time between 1-30 minutes; or
(d) the salt source is a concentrated salt solution, optionally wherein the salt concentration is between 0.0011\4 and 20 M; or
(e) the salt is an ionic halide; optionally wherein the salt is sodium chloride or magnesium chloride; or
(f) the detecting is performed by eye, using a spectrophotometer or by computer analysis; or
(g) the detecting is performed between 1-15 minutes after step (b); or
(h) the color change is from red or pink to blue or purple or colorless, optionally from red to blue;
(i) a red color indicates the virus nucleic acid sequence is present in the sample and a blue color indicates the virus nucleic acid sequence is not present in the sample; or
(j) the method is performed at between 18° C. to 30° C.

32. The method of claim 29, for detecting or diagnosing Zika virus infection in a patient.

33. The method of claim 29, wherein a kit comprises a probe comprising a metal particle and at least one polynucleotide comprising a sequence substantially complementary to a region within the non-structural 5 (NS5) gene of a virus of the Flaviviridae family.

34. The method of claim 26, further comprising a computing system configured to provide the user with the volume of the concentrated salt solution required to:
(a) increase the salt concentration of the composition comprising the particle and the polynucleotide to between 0.05-0.1 M;
(b) subsequently increase the salt concentration of the composition comprising the particle and the polynucleotide by 0.1-0.2 M; and
(c) subsequently increase the salt concentration of the composition comprising the particle and the polynucleotide according to step (b) between 5-10 times, such that the final salt concentration is between 0.6-1.0 M.

35. A device for use in detecting a Flaviviridae family virus or nucleic acid sequence thereof in a sample, the device comprising:
(a) a first chamber comprising a probe specific for a Flavividae family virus or nucleic acid sequence therefrom; and
(b) a second chamber comprising a salt source, wherein:
the device is configured to allow the probe to be contacted by the sample and, at a subsequent time, to allow the probe contacted by the sample to be contacted by the salt source.

36. The device of claim 35, configured to allow the probe to be contacted in the first chamber by the sample.

37. The device of claim 36, wherein:
(a) the device is configured to allow a third chamber containing the sample to be detachably connected to the first chamber;
(b) the device comprises a third chamber, the third chamber being configured to receive a sample; or
(c) the device comprises a third chamber, the third chamber comprising the sample.

38. The device of claim 37, wherein the third chamber is configured to
allow driving of at least a portion of the sample from the third chamber into the first chamber by increasing a pressure in the third chamber;
optionally configured to allow the pressure to be increased in the third chamber by reducing a volume of the third chamber;
optionally wherein the third chamber comprises a flexible wall and the reduction in volume is achieved via deformation of the flexible wall.

39. The device of claim 35, wherein the second chamber is configured to allow driving of at least a portion of the salt source from the second chamber into the first chamber by increasing a pressure in the second chamber;
optionally configured to allow the pressure to be increased in the second chamber by reducing a volume of the second chamber;
optionally wherein the second chamber comprises a flexible wall and the reduction in volume is achieved via deformation of the flexible wall.

40. The device of claim 35, further comprising a one-way valve to allow venting of gas out of the first chamber to prevent reflux of material from the first chamber into the second chamber or, where provided, the third chamber.

41. The device of claim 35, wherein:
(a) the first chamber is configured to allow optical inspection of material inside the first chamber through a wall of the first chamber; or
(b) the first chamber comprises a probe; comprising a metal particle and at least one polynucleotide comprising a sequence substantially complementary to a region within the non-structural 5 (NS5) gene of a virus of the Flaviviridae family; or
(c) the salt source is a concentrated salt solution; optionally wherein the concentrated salt solution comprises a salt concentration of between 0.001 M and 20 M; or
(d) the salt is an ionic halide, optionally sodium chloride.

42. A device of claim 35 which is adapted for or capable of performing a method of detecting a Flaviviridae family virus or nucleic acid sequence thereof in a sample, the method comprising:
(a) contacting a probe or a polynucleotide with the sample; wherein the probe comprises a metal particle and at least one polynucleotide comprising a sequence substantially complementary to a region within the non-structural 5 (NS5) gene of a virus of the Flavivitidae family; wherein the polynucleotide comprises a sequence substantially complementary to a region within the non-structural 5 (NS5) gene of a virus of the Flaviviridae family;
(b) contacting the composition resulting from step (a) with a salt source; and
(c) optionally detecting either:
(i) a color change if the viral nucleic acid sequence is not present in the sample; or
(ii) no color change if the viral nucleic acid sequence is present in the sample.

43. (canceled)

44. (canceled)

45. (canceled)

46. (canceled)

47. The probe of claim 1, wherein the polynucleotide:
(a) has a length of between 10 to 30 nucleotides and
(b) further comprises a thiol group at the 3′ or 5′ terminus.

48. The probe of claim 1, wherein the metal particle:
(a) comprises gold;
(c) is substantially spherical; and
(d) has a diameter between 10 nm to 20 nm.

49. The probe of claim 1, wherein:
(a) the at least one polynucleotide is linked to the particle through a thiol group at the 3′ or 5′ terminus of the polynucleotide;
(b) the particle is conjugated to a plurality of polynucleotides;
(c) the probe comprises between 100-200 copies of the polynucleotide; and
(d) the density of polynucleotides on the probe is between 20-40 pmol/cm².

50. (canceled)

51. (canceled)

52. The method of claim 26, wherein:
(a) the salt source is a concentrated salt solution;
(b) the salt is an ionic halide; and
(c) increasing the salt concentration stepwise comprises increasing the salt concentration by up to between 0.1-0.2 M every at least 15 minutes.

53. The method of claim 29, wherein:
(a) the virus is Zika virus;
(b) the sample is a biological sample or a sample from a patient; optionally wherein:)
(i) the sample from a patient comprises urine, blood, saliva or cells; or
(ii) the biological sample comprises or is derived from mosquito cells or tick cells;
(c) the sample is used directly in the method;
(d) the sample is a liquid.

54. The method of claim 29, wherein:
(a) the probe is in solution or suspension, optionally at a concentration between 0.5 and 50 nM;
(b) the probe is adhered or dried onto a solid support or matrix;
(c) the contacting occurs over a period of time between 1-30 minutes;
(d) the salt source is a concentrated salt solution, optionally wherein the salt concentration is between 0.001 M and 20 M;
(e) the salt is an ionic halide; optionally wherein the salt is sodium chloride or magnesium chloride;
(f) the detecting is performed by eye, using a spectrophotometer or by computer analysis;
(g) the detecting is performed between 1-15 minutes after step (b);
(h) the color change is from red or pink to blue or purple or colorless, optionally from red to blue;
(i) a red color indicates the virus nucleic acid sequence is present in the sample and a blue color indicates the virus nucleic acid sequence is not present in the sample; and
(j) the method is performed at between 18° C. to 30° C.

55. The probe of claim 4, wherein the polynucleotide further comprises a linker between the polynucleotide and the thiol group at the 3′ or 5′ terminus;

56. The probe of claim 56, wherein the linker comprises 5 to 10 nucleotides, optionally wherein the 5 to 10 nucleotides of the linker are at least 80% A and T nucleotides,

57. The composition according to claim 10, wherein
(a) The composition comprises a sample; or
(b) The composition comprises a biological sample or a sample from a patient; or
(c) The composition comprises a sample from a patient; wherein the sample from a patient comprises urine, blood, saliva or cells; or
(d) The composition comprises a biological sample wherein the biological sample comprises or is derived from mosquito cells or tick cells.

58. The method of claim 28, wherein
(a) the concentrated salt solution comprises a salt concentration of between 2 M and 20M; or
(b) the ionic halide is sodium chloride.

59. The method of claim 30, wherein
(i) the sample from the patient comprises urine, blood, saliva or cells; or
(ii) the biological sample comprises or is derived from mosquito cells or tick cells;

60. The method of claim 30, further comprising
(a) treating the sample prior to use;
(b) purifying total DNA, RNA or both in the sample and
(c) amplifying the total DNA, RNA or both in the sample or amplifying specific viral nucleic acid sequences in the sample.

61. The method of claim60, wherein the amplification is performed by PCR, RT-PCR, rRT-PCR, NASBA, LAMP or WA.

62. The probe of claim 47, wherein the polynucleotide further comprises a linker between the polynucleotide and the thiol group at the 3′ or 5′ terminus.

63. The probe of claim 62, wherein
the linker comprises 5 to 10 nucleotides, or
the linker comprises 5 to 10 nucleotides, and the 5 to 10 nucleotides of the linker are at least 80% A and T nucleotides.

64. The method of claim 29, further comprising
(a) treating the sample prior to use;
(b) purifying total DNA, RNA or both in the sample and
(c) amplifying the total DNA, RNA or both in the sample or amplifying specific viral nucleic acid sequences in the sample.

65. The method of claim 64, wherein the amplification is performed by PCR, RT-PCR, rRT-PCR, NASBA, LAMP or HCR.


SEQUENCE LISTING (17 sequences)

SEQ ID NO: 1 - DNA, 10794 bp, Flavivirus Zika virus
agttgttgatctgtgtgagtcagactgcgacagttcgagtctgaagcgagagctaacaacagtatcaacaggtttaatttggatttggaaacgagagtttctggtcatgaaaaaccccaaagaagaaatccggaggatccggattgtcaatatgctaaaacgcggagtagcccgtgtaaaccccttgggaggtttgaagaggttgccagccggacttctgctgggtcatggacccatcagaatggttttggcgatactagcctttttgagatttacagcaatcaagccatcactgggccttatcaacagatggggttccgtggggaaaaaagaggctatggaaataataaagaagttcaagaaagatcttgctgccatgttgagaataatcaatgctaggaaagagaggaagagacgtggcgcagacaccagcatcggaatcattggcctcctgctgactacagccatggcagcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagtatcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggactttttggcaaagggagcttggtgacatgtgccaagtttacgtgttctaagaagatgaccgggaagagcattcaaccggaaaatctggagtatcggataatgctatcagtgcatggctcccagcatagcgggatgattggatatgaaactgacgaagatagagcgaaagtcgaggttacgcctaattcaccaagagcggaagcaaccttgggaggctttggaagcttaggacttgactgtgaaccaaggacaggccttgacttttcagatctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaggctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacatgcttggccctggggggagtgatgatcttcctctccacggctgtttctgctgacgtggggtgctcagtggacttctcaaaaaaggaaacgagatgtggcacgggggtattcatctataatgatgttgaagcctggagggaccggtacaagtaccatcctgactccccccgcagattggcagcagcagtcaagcaggcctgggaagaggggatctgtgggatctcatccgtttcaagaatggaaaacatcatgtggaaatcagtagaaggggagctcaatgctatcctagaggagaatggagttcaactgacagttgttgtgggatctgtaaaaaaccccatgtggagaggtccacaaagattgccagtgcctgtgaatgagctgccccatggctggaaagcctgggggaaatcgtattttgttagggcggcaaagaccaacaacagttttgttgtcgacggtgacacactgaaggaatgtccgcttgagcacagagcatggaatagttttcttgtggaggatcacgggtttggagtcttccacaccagtgtctggcttaaggtcagagaagattactcattagaatgtgacccagccgtcataggaacagctgttaagggaagggaggccgcgcacagtgatctgggctattggattgaaagtgaaaagaatgacacatggaggctgaagagggcccacctgattgagatgaaaacatgtgaatggccaaagtctcacacattgtggacagatggagtagaagaaagtgatcttatcatacccaagtctttagctggtccactcagccaccacaacaccagagagggttacagaacccaagtgaaagggccatggcacagtgaagagcttgaaatccggtttgaggaatgtccaggcaccaaggtttacgtggaggagacatgcggaactagaggaccatctctgagatcaactactgcaagtggaagggtcattgaggaatggtgctgtagggaatgcacaatgcccccactatcgtttcgagcaaaagacggctgctggtatggaatggagataaggcccaggaaagaaccagagagcaacttagtgaggtcaatggtgacagcggggtcaaccgatcatatggaccacttctctcttggagtgcttgtgattctactcatggtgcaggaggggttgaagaagagaatgaccacaaagatcatcatgagcacatcaatggcagtgctggtagtcatgatcttgggaggattttcaatgagtgacctggccaagcttgtgatcctgatgggtgctactttcgcagaaatgaacactggaggagatgtagctcacttggcattggtagcggcatttaaagtcagaccagccttgctggtctccttcattttcagagccaattggacaccccgtgagagcatgctgctagccctggcttcgtgtcttctgcaaactgcgatctctgctcttgaaggtgacttgatggtcctcattaatggatttgctttggcctggttggcaattcgagcaatggccgtgccacgcactgacaacatcgctctaccaatcttggctgctctaacaccactagctcgaggcacactgctcgtggcatggagagcgggcctggctacttgtggagggatcatgctcctctccctgaaagggaaaggtagtgtgaagaagaacctgccatttgtcatggccctgggattgacagctgtgagggtagtagaccctattaatgtggtaggactactgttactcacaaggagtgggaagcggagctggccccctagtgaagttctcacagccgttggcctgatatgtgcactggccggagggtttgccaaggcagacattgagatggctggacccatggctgcagtaggcttgctaattgtcagctatgtggtctcgggaaagagtgtggacatgtacattgaaagagcaggtgacatcacatgggaaaaggacgcggaagtcactggaaacagtcctcggcttgacgtggcactggatgagagtggtgacttctccttggtagaggaagatggtccacccatgagagagatcatactcaaggtggtcctgatggccatctgtggcatgaacccaatagctataccttttgctgcaggagcgtggtatgtgtatgtgaagactgggaaaaggagtggcgccctctgggacgtgcctgctcccaaagaagtgaagaaaggagagaccacagatggagtgtacagagtgatgactcgcagactgctaggttcaacacaggttggagtgggagtcatgcaagagggagtcttccacaccatgtggcacgttacaaaaggagccgcactgaggagcggtgagggaagacttgatccatactggggggatgtcaagcaggacttggtgtcatactgtgggccttggaagttggatgcagcttgggatggactcagcgaggtacagcttttggccgtacctcccggagagagggccagaaacattcagaccctgcctggaatattcaagacaaaggacggggacatcggagcagttgctctggactaccctgcagggacctcaggatctccgatcctagacaaatgtggaagagtgataggactctatggcaatggggttgtgatcaagaatggaagctatgttagtgctataacccagggaaagagggaggaggagactccggttgaatgtttcgaaccctcgatgctgaagaagaagcagctaactgtcttggatctgcatccaggagccggaaaaaccaggagagttcttcctgaaatagtccgtgaagccataaaaaagagactccggacagtgatcttggcaccaactagggttgtcgctgctgagatggaggaggccttgagaggacttccggtgcgttacatgacaacagcagtcaacgtcacccattctgggacagaaatcgttgatttgatgtgccatgccactttcacttcacgcttactacaacccatcagagtccctaattacaatctcaacatcatggatgaagcccacttcacagacccctcaagtatagctgcaagaggatacatatcaacaagggttgaaatgggcgaggcggctgccatttttatgactgccacaccaccaggaacccgtgatgcgtttcctgactctaactcaccaatcatggacacagaagtggaagtcccagagagagcctggagctcaggctttgattgggtgacagaccattctgggaaaacagtttggttcgttccaagcgtgagaaacggaaatgaaatcgcagcctgtctgacaaaggctggaaagcgggtcatacagctcagcaggaagacttttgagacagaatttcagaaaacaaaaaatcaagagtgggactttgtcataacaactgacatctcagagatgggcgccaacttcaaggctgaccgggtcatagactctaggagatgcctaaaaccagtcatacttgatggtgagagagtcatcttggctgggcccatgcctgtcacgcatgctagtgctgctcagaggagaggacgtataggcaggaaccctaacaaacctggagatgagtacatgtatggaggtgggtgtgcagagactgatgaaggccatgcacactggcttgaagcaagaatgcttcttgacaacatctacctccaggatggcctcatagcctcgctctatcggcctgaggccgataaggtagccgccattgagggagagtttaagctgaggacagagcaaaggaagaccttcgtggaactcatgaagagaggagaccttcccgtctggctagcctatcaggttgcatctgccggaataacttacacagacagaagatggtgctttgatggcacaaccaacaacaccataatggaagacagtgtaccagcagaggtttggacaaagtatggagagaagagagtgctcaaaccgagatggatggatgctagggtctgttcagaccatgcggccctgaagtcgttcaaagaattcgccgctggaaaaagaggagcggctttgggagtaatggaggccctgggaacactgccaggacacatgacagagaggtttcaggaagccattgacaacctcgccgtgctcatgcgagcagagactggaagcaggccttataaggcagcggcagcccaactgccggagaccctagagaccattatgctcttaggtttgctgggaacagtttcactggggatcttcttcgtcttgatgcggaataagggcatcgggaagatgggctttggaatggtaacccttggggccagtgcatggctcatgtggctttcggaaattgaaccagccagaattgcatgtgtcctcattgttgtgtttttattactggtggtgctcatacccgagccagagaagcaaagatctccccaagataaccagatggcaattatcatcatggtggcagtgggccttctaggtttgataactgcaaacgaacttggatggctggaaagaacaaaaaatgacatagctcatctaatgggaaggagagaagaaggagcaaccatgggattctcaatggacattgatctgcggccagcctccgcctgggctatctatgccgcattgacaactctcatcaccccagctgtccaacatgcggtaaccacttcatacaacaactactccttaatggcgatggccacacaagctggagtgctgtttggcatgggcaaagggatgccatttatgcatggggaccttggagtcccgctgctaatgatgggttgctattcacaattaacacccctgactctgatagtagctatcattctgcttgtggcgcactacatgtacttgatcccaggcctacaagcggcagcagcgcgtgctgcccagaaaaggacagcagctggcatcatgaagaatcccgttgtggatggaatagtggtaactgacattgacacaatgacaatagacccccaggtggagaagaagatgggacaagtgttactcatagcagtagccatctccagtgctgtgctgctgcggaccgcctggggatggggggaggctggagctctgatcacagcagcgacctccaccttgtgggaaggctctccaaacaaatactggaactcctctacagccacctcactgtgcaacatcttcagaggaagctatctggcaggagcttcccttatctatacagtgacgagaaacgctggcctggttaagagacgtggaggtgggacgggagagactctgggagagaagtggaaagctcgtctgaatcagatgtcggccctggagttctactcttataaaaagtcaggtatcactgaagtgtgtagagaggaggctcgccgtgccctcaaggatggagtggccacaggaggacatgccgtatcccggggaagtgcaaagatcagatggttggaggagagaggatatctgcagccctatgggaaggttgttgacctcggatgtggcagagggggctggagctattatgccgccaccatccgcaaagtgcaggaggtgagaggatacacaaagggaggtcccggtcatgaagaacccatgctggtgcaaagctatgggtggaacatagttcgtctcaagagtggagtggacgtcttccacatggcggctgagccgtgtgacactctgctgtgtgacataggtgagtcatcatctagtcctgaagtggaagagacacgaacactcagagtgctctctatggtgggggactggcttgaaaaaagaccaggggccttctgtataaaggtgctgtgcccatacaccagcactatgatggaaaccatggagcgactgcaacgtaggcatgggggaggattagtcagagtgccattgtgtcgcaactccacacatgagatgtactgggtctctggggcaaagagcaacatcataaaaagtgtgtccaccacaagtcagctcctcctgggacgcatggatggccccaggaggccagtgaaatatgaggaggatgtgaacctcggctcgggtacacgagctgtggcaagctgtgctgaggctcctaacatgaaaatcatcggcaggcgcattgagagaatccgcaatgaacatgcagaaacatggtttcttgatgaaaaccacccatacaggacatgggcctaccatgggagctacgaagcccccacgcaaggatcagcgtcttccctcgtgaacggggttgttagactcctgtcaaagccttgggacgtggtgactggagttacaggaatagccatgactgacaccacaccatacggccaacaaagagtcttcaaagaaaaagtggacaccagggtgccagatccccaagaaggcactcgccaggtaatgaacatagtctcttcctggctgtggaaggagctggggaaacgcaagcggccacgcgtctgcaccaaagaagagtttatcaacaaggtgcgcagcaatgcagcactgggagcaatatttgaagaggaaaaagaatggaagacggctgtggaagctgtgaatgatccaaggttttgggccctagtggatagggagagagaacaccacctgagaggagagtgtcacagctgtgtgtacaacatgatgggaaaaagagaaaagaagcaaggagagttcgggaaagcaaaaggtagccgcgccatctggtacatgtggttgggagccagattcttggagtttgaagcccttggattcttgaacgaggaccattggatgggaagagaaaactcaggaggtggagtcgaagggttaggattgcaaagacttggatacattctagaagaaatgaatcgggcaccaggaggaaagatgtacgcagatgacactgctggctgggacacccgcattagtaagtttgatctggagaatgaagctctgattaccaaccaaatggaggaagggcacagaactctggcgttggccgtgattaaatacacataccaaaacaaagtggtgaaggttctcagaccagctgaaggaggaaaaacagttatggacatcatttcaagacaagaccagagagggagtggacaagttgtcacttatgctctcaacacattcaccaacttggtggtgcagcttatccggaacatggaagctgaggaagtgttagagatgcaagacttatggttgttgaggaagccagagaaagtgaccagatggttgcagagcaatggatgggatagactcaaacgaatggcggtcagtggagatgactgcgttgtgaagccaatcgatgataggtttgcacatgccctcaggttcttgaatgacatgggaaaagttaggaaagacacacaggagtggaaaccctcgactggatggagcaattgggaagaagtcccgttctgctcccaccacttcaacaagctgtacctcaaggatgggagatccattgtggtcccttgccgccaccaagatgaactgattggccgagctcgcgtctcaccaggggcaggatggagcatccgggagactgcctgtcttgcaaaatcatatgcgcagatgtggcagctcctttatttccacagaagagaccttcgactgatggctaatgccatttgctcggctgtgccagttgactgggtaccaactgggagaaccacctggtcaatccatggaaagggagaatggatgaccactgaggacatgctcatggtgtggaatagagtgtggattgaggagaacgaccatatggaggacaagactcctgtaacaaaatggacagacattccctatctaggaaaaagggaggacttatggtgtggatcccttatagggcacagaccccgcaccacttgggctgaaaacatcaaagacacagtcaacatggtgcgcaggatcataggtgatgaagaaaagtacatggactatctatccacccaagtccgctacttgggtgaggaagggtccacacccggagtgttgtaagcaccaattttagtgttgtcaggcctgctagtcagccacagtttggggaaagctgtgcagcctgtaacccccccaggagaagctgggaaaccaagctcatagtcaggccgagaacgccatggcacggaagaagccatgctgcctgtgagcccctcagaggacactgagtcaaaaaaccccacgcgcttggaagcgcaggatgggaaaagaaggtggcgaccttccccacccttcaatctggggcctgaactggagactagctgtgaatctccagcagagggactagtggttagaggagaccccccggaaaacgcaaaacagcatattgacgtgggaaagaccagagactccatgagtttccaccacgctggccgccaggcacagatcgccgaacttcggcggccggtgtggggaaatccatggtttct

SEQ ID NO: 2 - DNA, 1500 bp, Flavivirus Zika virus
atcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggactttttggcaaagggagcttggtgacatgtgccaagtttacgtgttctaagaagatgaccgggaagagcattcaaccggaaaatctggagtatcggataatgctatcagtgcatggctcccagcatagcgggatgattggatatgaaactgacgaagatagagcgaaagtcgaggttacgcctaattcaccaagagcggaagcaaccttgggaggctttggaagcttaggacttgactgtgaaccaaggacaggccttgacttttcagatctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaggctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacatgcttggccctggggggagtgatgatcttcctctccacggctgtttctgct

SEQ ID NO: 3 - DNA, 10363 bp, Flavivirus Zika virus
agttgttgatctgtgtgagtcagactgcgacagttcgagtctgaagcgagagctaacaacagtatcaacaggtttaatttggatttggaaacgagagtttctggtcatgaaaaaccccaaagaagaaatccggaggatccggattgtcaatatgctaaaacgcggagtagcccgtgtaaaccccttgggaggtttgaagaggttgccagccggacttctgctgggtcatggacccatcagaatggttttggcgatactagcctttttgagatttacagcaatcaagccatcactgggccttatcaacagatggggttccgtggggaaaaaagaggctatggaaataataaagaagttcaagaaagatcttgctgccatgttgagaataatcaatgctaggaaagagaggaagagacgtggcgcagacaccagcatcggaatcattggcctcctgctgactacagccatggcagcagagatcactagacgcgggagtgcatactacatgtacttggataggagcgatgccgggaaggccatttcgtttgctaccacattgggagtgaacaagtgccacgtacagatcatggacctcgggcacatgtgtgacgccaccatgagttatgagtgccctatgctggatgagggagtggaaccagatgatgtcgattgctggtgcaacacgacatcaacttgggttgtgtacggaacctgtcatcacaaaaaaggtgaggcacggcgatctagaagagccgtgacgctcccttctcactctacaaggaagttgcaaacgcggtcgcagacctggttagaatcaagagaatacacgaagcacttgatcaaggttgaaaactggatattcaggaaccccgggtttgcgctagtggccgttgccattgcctggcttttgggaagctcgacgagccaaaaagtcatatacttggtcatgatactgctgattgccccggcatacagtatcaggtgcattggagtcagcaatagagacttcgtggagggcatgtcaggtgggacctgggttgatgttgtcttggaacatggaggctgcgttaccgtgatggcacaggacaagccaacagtcgacatagagttggtcacgacgacggttagtaacatggccgaggtaagatcctattgctacgaggcatcgatatcggacatggcttcggacagtcgttgcccaacacaaggtgaagcctaccttgacaagcaatcagacactcaatatgtctgcaaaagaacattagtggacagaggttggggaaacggttgtggactttttggcaaagggagcttggtgacatgtgccaagtttacgtgttctaagaagatgaccgggaagagcattcaaccggaaaatctggagtatcggataatgctatcagtgcatggctcccagcatagcgggatgattggatatgaaactgacgaagatagagcgaaagtcgaggttacgcctaattcaccaagagcggaagcaaccttgggaggctttggaagcttaggacttgactgtgaaccaaggacaggccttgacttttcagatctgtattacctgaccatgaacaataagcattggttggtgcacaaagagtggtttcatgacatcccattgccttggcatgctggggcagacaccggaactccacactggaacaacaaagaggcattggtagaattcaaggatgcccacgccaagaggcaaaccgtcgtcgttctggggagccaggaaggagccgttcacacggctctcgctggagctctagaggctgagatggatggtgcaaagggaaggctgttctctggccatttgaaatgccgcctaaaaatggacaagcttagattgaagggcgtgtcatattccttgtgcactgcggcattcacattcaccaaggtcccagctgaaacactgcatggaacagtcacagtggaggtgcagtatgcagggacagatggaccctgcaagatcccagtccagatggcggtggacatgcagaccctgaccccagttggaaggctgataaccgccaaccccgtgattactgaaagcactgagaactcaaagatgatgttggagcttgacccaccatttggggattcttacattgtcataggagttggggacaagaaaatcacccaccactggcataggagtggtagcaccatcggaaaggcatttgaggccactgtgagaggcgccaagagaatggcagtcctgggggatacagcctgggacttcggatcagtcgggggtgtgttcaactcactgggtaagggcattcaccagatttttggagcagccttcaaatcactgtttggaggaatgtcctggttctcacagatcctcataggcacgctgctagtgtggttaggtttgaacacaaagaatggatctatctccctcacatgcttggccctggggggagtgatgatcttcctctccacggctgtttctgctgacgtggggtgctcagtggacttctcaaaaaaggaaacgagatgtggcacgggggtattcatctataatgatgttgaagcctggagggaccggtacaagtaccatcctgactccccccgcagattggcagcagcagtcaagcaggcctgggaagaggggatctgtgggatctcatccgtttcaagaatggaaaacatcatgtggaaatcagtagaaggggagctcaatgctatcctagaggagaatggagttcaactgacagttgttgtgggatctgtaaaaaaccccatgtggagaggtccacaaagattgccagtgcctgtgaatgagctgccccatggctggaaagcctgggggaaatcgtattttgttagggcggcaaagaccaacaacagttttgttgtcgacggtgacacactgaaggaatgtccgcttgagcacagagcatggaatagttttcttgtggaggatcacgggtttggagtcttccacaccagtgtctggcttaaggtcagagaagattactcattagaatgtgacccagccgtcataggaacagctgttaagggaagggaggccgcgcacagtgatctgggctattggattgaaagtgaaaagaatgacacatggaggctgaagagggcccacctgattgagatgaaaacatgtgaatggccaaagtctcacacattgtggacagatggagtagaagaaagtgatcttatcatacccaagtctttagctggtccactcagccaccacaacaccagagagggttacagaacccaagtgaaagggccatggcacagtgaagagcttgaaatccggtttgaggaatgtccaggcaccaaggtttacgtggaggagacatgcggaactagaggaccatctctgagatcaactactgcaagtggaagggtcattgaggaatggtgctgtagggaatgcacaatgcccccactatcgtttcgagcaaaagacggctgctggtatggaatggagataaggcccaggaaagaaccagagagcaacttagtgaggtcaatggtgacagcggggtcaaccgatcatatggaccacttctctcttggagtgcttgtgattctactcatggtgcaggaggggttgaagaagagaatgaccacaaagatcatcatgagcacatcaatggcagtgctggtagtcatgatcttgggaggattttcaatgagtgacctggccaagcttgtgatcctgatgggtgctactttcgcagaaatgaacactggaggagatgtagctcacttggcattggtagcggcatttaaagtcagaccagccttgctggtctccttcattttcagagccaattggacaccccgtgagagcatgctgctagccctggcttcgtgtcttctgcaaactgcgatctctgctcttgaaggtgacttgatggtcctcattaatggatttgctttggcctggttggcaattcgagcaatggccgtgccacgcactgacaacatcgctctaccaatcttggctgctctaacaccactagctcgaggcacactgctcgtggcatggagagcgggcctggctacttgtggagggatcatgctcctctccctgaaagggaaaggtagtgtgaagaagaacctgccatttgtcatggccctgggattgacagctgtgagggtagtagaccctattaatgtggtaggactactgttactcacaaggagtgggaagcggagctggccccctagtgaagttctcacagccgttggcctgatatgtgcactggccggagggtttgccaaggcagacattgagatggctggacccatggctgcagtaggcttgctaattgtcagctatgtggtctcgggaaagagtgtggacatgtacattgaaagagcaggtgacatcacatgggaaaaggacgcggaagtcactggaaacagtcctcggcttgacgtggcactggatgagagtggtgacttctccttggtagaggaagatggtccacccatgagagagatcatactcaaggtggtcctgatggccatctgtggcatgaacccaatagctataccttttgctgcaggagcgtggtatgtgtatgtgaagactgggaaaaggagtggcgccctctgggacgtgcctgctcccaaagaagtgaagaaaggagagaccacagatggagtgtacagagtgatgactcgcagactgctaggttcaacacaggttggagtgggagtcatgcaagagggagtcttccacaccatgtggcacgttacaaaaggagccgcactgaggagcggtgagggaagacttgatccatactggggggatgtcaagcaggacttggtgtcatactgtgggccttggaagttggatgcagcttgggatggactcagcgaggtacagcttttggccgtacctcccggagagagggccagaaacattcagaccctgcctggaatattcaagacaaaggacggggacatcggagcagttgctctggactaccctgcagggacctcaggatctccgatcctagacaaatgtggaagagtgataggactctatggcaatggggttgtgatcaagaatggaagctatgttagtgctataacccagggaaagagggaggaggagactccggttgaatgtttcgaaccctcgatgctgaagaagaagcagctaactgtcttggatctgcatccaggagccggaaaaaccaggagagttcttcctgaaatagtccgtgaagccataaaaaagagactccggacagtgatcttggcaccaactagggttgtcgctgctgagatggaggaggccttgagaggacttccggtgcgttacatgacaacagcagtcaacgtcacccattctgggacagaaatcgttgatttgatgtgccatgccactttcacttcacgcttactacaacccatcagagtccctaattacaatctcaacatcatggatgaagcccacttcacagacccctcaagtatagctgcaagaggatacatatcaacaagggttgaaatgggcgaggcggctgccatttttatgactgccacaccaccaggaacccgtgatgcgtttcctgactctaactcaccaatcatggacacagaagtggaagtcccagagagagcctggagctcaggctttgattgggtgacagaccattctgggaaaacagtttggttcgttccaagcgtgagaaacggaaatgaaatcgcagcctgtctgacaaaggctggaaagcgggtcatacagctcagcaggaagacttttgagacagaatttcagaaaacaaaaaatcaagagtgggactttgtcataacaactgacatctcagagatgggcgccaacttcaaggctgaccgggtcatagactctaggagatgcctaaaaccagtcatacttgatggtgagagagtcatcttggctgggcccatgcctgtcacgcatgctagtgctgctcagaggagaggacgtataggcaggaaccctaacaaacctggagatgagtacatgtatggaggtgggtgtgcagagactgatgaaggccatgcacactggcttgaagcaagaatgcttcttgacaacatctacctccaggatggcctcatagcctcgctctatcggcctgaggccgataaggtagccgccattgagggagagtttaagctgaggacagagcaaaggaagaccttcgtggaactcatgaagagaggagaccttcccgtctggctagcctatcaggttgcatctgccggaataacttacacagacagaagatggtgctttgatggcacaaccaacaacaccataatggaagacagtgtaccagcagaggtttggacaaagtatggagagaagagagtgctcaaaccgagatggatggatgctagggtctgttcagaccatgcggccctgaagtcgttcaaagaattcgccgctggaaaaagaggagcggctttgggagtaatggaggccctgggaacactgccaggacacatgacagagaggtttcaggaagccattgacaacctcgccgtgctcatgcgagcagagactggaagcaggccttataaggcagcggcagcccaactgccggagaccctagagaccattatgctcttaggtttgctgggaacagtttcactggggatcttcttcgtcttgatgcggaataagggcatcgggaagatgggctttggaatggtaacccttggggccagtgcatggctcatgtggctttcggaaattgaaccagccagaattgcatgtgtcctcattgttgtgtttttattactggtggtgctcatacccgagccagagaagcaaagatctccccaagataaccagatggcaattatcatcatggtggcagtgggccttctaggtttgataactgcaaacgaacttggatggctggaaagaacaaaaaatgacatagctcatctaatgggaaggagagaagaaggagcaaccatgggattctcaatggacattgatctgcggccagcctccgcctgggctatctatgccgcattgacaactctcatcaccccagctgtccaacatgcggtaaccacttcatacaacaactactccttaatggcgatggccacacaagctggagtgctgtttggcatgggcaaagggatgccatttatgcatggggaccttggagtcccgctgctaatgatgggttgctattcacaattaacacccctgactctgatagtagctatcattctgcttgtggcgcactacatgtacttgatcccaggcctacaagcggcagcagcgcgtgctgcccagaaaaggacagcagctggcatcatgaagaatcccgttgtggatggaatagtggtaactgacattgacacaatgacaatagacccccaggtggagaagaagatgggacaagtgttactcatagcagtagccatctccagtgctgtgctgctgcggaccgcctggggatggggggaggctggagctctgatcacagcagcgacctccaccttgtgggaaggctctccaaacaaatactggaactcctctacagccacctcactgtgcaacatcttcagaggaagctatctggcaggagcttcccttatctatacagtgacgagaaacgctggcctggttaagagacgtggaggtgggacgggagagactctgggagagaagtggaaagctcgtctgaatcagatgtcggccctggagttctactcttataaaaagtcaggtatcactgaagtgtgtagagaggaggctcgccgtgccctcaaggatggagtggccacaggaggacatgccgtatcccggggaagtgcaaagatcagatggttggaggagagaggatatctgcagccctatgggaaggttgttgacctcggatgtggcagagggggctggagctattatgccgccaccatccgcaaagtgcaggaggtgagaggatacacaaagggaggtcccggtcatgaagaacccatgctggtgcaaagctatgggtggaacatagttcgtctcaagagtggagtggacgtcttccacatggcggctgagccgtgtgacactctgctgtgtgacataggtgagtcatcatctagtcctgaagtggaagagacacgaacactcagagtgctctctatggtgggggactggcttgaaaaaagaccaggggccttctgtataaaggtgctgtgcccatacaccagcactatgatggaaaccatggagcgactgcaacgtaggcatgggggaggattagtcagagtgccattgtgtcgcaactccacacatgagatgtactgggtctctggggcaaagagcaacatcataaaaagtgtgtccaccacaagtcagctcctcctgggacgcatggatggccccaggaggccagtgaaatatgaggaggatgtgaacctcggctcgggtacacgagctgtggcaagctgtgctgaggctcctaacatgaaaatcatcggcaggcgcattgagagaatccgcaatgaacatgcagaaacatggtttcttgatgaaaaccacccatacaggacatgggcctaccatgggagctacgaagcccccacgcaaggatcagcgtcttccctcgtgaacggggttgttagactcctgtcaaagccttgggacgtggtgactggagttacaggaatagccatgactgacaccacaccatacggccaacaaagagtcttcaaagaaaaagtggacaccagggtgccagatccccaagaaggcactcgccaggtaatgaacatagtctcttcctggctgtggaaggagctggggaaacgcaagcggccacgcgtctgcaccaaagaagagtttatcaacaaggtgcgcagcaatgcagcactgggagcaatatttgaagaggaaaaagaatggaagacggctgtggaagctgtgaatgatccaaggttttgggccctagtggatagggagagagaacaccacctgagaggagagtgtcacagctgtgtgtacaacatgatgggaaaaagagaaaagaagcaaggagagttcgggaaagcaaaaggtagccgcgccatctggtacatgtggttgggagccagattcttggagtttgaagcccttggattcttgaacgaggaccattggatgggaagagaaaactcaggaggtggagtcgaagggttaggattgcaaagacttggatacattctagaagaaatgaatcgggcaccaggaggaaagatgtacgcagatgacactgctggctgggacacccgcattagtaagtttgatctggagaatgaagctctgattaccaaccaaatggaggaagggcacagaactctggcgttggccgtgattaaatacacataccaaaacaaagtggtgaaggttctcagaccagctgaaggaggaaaaacagttatggacatcatttcaagacaagaccagagagggagtggacaagttgtcacttatgctctcaacacattcaccaacttggtggtgcagcttatccggaacatggaagctgaggaagtgttagagatgcaagacttatggttgttgaggaagccagagaaagtgaccagatggttgcagagcaatggatgggatagactcaaacgaatggcggtcagtggagatgactgcgttgtgaagccaatcgatgataggtttgcacatgccctcaggttcttgaatgacatgggaaaagttaggaaagacacacaggagtggaaaccctcgactggatggagcaattgggaagaagtcccgttctgctcccaccacttcaacaagctgtacctcaaggatgggagatccattgtggtcccttgccgccaccaagatgaactgattggccgagctcgcgtctcaccaggggcaggatggagcatccgggagactgcctgtcttgcaaaatcatatgcgcagatgtggcagctcctttatttccacagaagagaccttcgactgatggctaatgccatttgctcggctgtgccagttgactgggtaccaactgggagaaccacctggtcaatccatggaaagggagaatggatgaccactgaggacatgctcatggtgtggaatagagtgtggattgaggagaacgaccatatggaggacaagactcctgtaacaaaatggacagacattccctatctaggaaaaagggaggacttatggtgtggatcccttatagggcacagaccccgcaccacttgggctgaaaacatcaaagacacagtcaacatggtgcgcaggatcataggtgatgaagaaaagtacatggactatctatccacccaagtccgctacttgggtgaggaagggtccacacccggagtgttg

SEQ ID NO: 4 - DNA, 1851 bp, Flavivirus Zika virus
agtggcgccctctgggacgtgcctgctcccaaagaagtgaagaaaggagagaccacagatggagtgtacagagtgatgactcgcagactgctaggttcaacacaggttggagtgggagtcatgcaagagggagtcttccacaccatgtggcacgttacaaaaggagccgcactgaggagcggtgagggaagacttgatccatactggggggatgtcaagcaggacttggtgtcatactgtgggccttggaagttggatgcagcttgggatggactcagcgaggtacagcttttggccgtacctcccggagagagggccagaaacattcagaccctgcctggaatattcaagacaaaggacggggacatcggagcagttgctctggactaccctgcagggacctcaggatctccgatcctagacaaatgtggaagagtgataggactctatggcaatggggttgtgatcaagaatggaagctatgttagtgctataacccagggaaagagggaggaggagactccggttgaatgtttcgaaccctcgatgctgaagaagaagcagctaactgtcttggatctgcatccaggagccggaaaaaccaggagagttcttcctgaaatagtccgtgaagccataaaaaagagactccggacagtgatcttggcaccaactagggttgtcgctgctgagatggaggaggccttgagaggacttccggtgcgttacatgacaacagcagtcaacgtcacccattctgggacagaaatcgttgatttgatgtgccatgccactttcacttcacgcttactacaacccatcagagtccctaattacaatctcaacatcatggatgaagcccacttcacagacccctcaagtatagctgcaagaggatacatatcaacaagggttgaaatgggcgaggcggctgccatttttatgactgccacaccaccaggaacccgtgatgcgtttcctgactctaactcaccaatcatggacacagaagtggaagtcccagagagagcctggagctcaggctttgattgggtgacagaccattctgggaaaacagtttggttcgttccaagcgtgagaaacggaaatgaaatcgcagcctgtctgacaaaggctggaaagcgggtcatacagctcagcaggaagacttttgagacagaatttcagaaaacaaaaaatcaagagtgggactttgtcataacaactgacatctcagagatgggcgccaacttcaaggctgaccgggtcatagactctaggagatgcctaaaaccagtcatacttgatggtgagagagtcatcttggctgggcccatgcctgtcacgcatgctagtgctgctcagaggagaggacgtataggcaggaaccctaacaaacctggagatgagtacatgtatggaggtgggtgtgcagagactgatgaaggccatgcacactggcttgaagcaagaatgcttcttgacaacatctacctccaggatggcctcatagcctcgctctatcggcctgaggccgataaggtagccgccattgagggagagtttaagctgaggacagagcaaaggaagaccttcgtggaactcatgaagagaggagaccttcccgtctggctagcctatcaggttgcatctgccggaataacttacacagacagaagatggtgctttgatggcacaaccaacaacaccataatggaagacagtgtaccagcagaggtttggacaaagtatggagagaagagagtgctcaaaccgagatggatggatgctagggtctgttcagaccatgcggccctgaagtcgttcaaagaattcgccgctggaaaaaga

SEQ ID NO: 5 - DNA, 2709 bp, Flavivirus Zika virus
ggaggtgggacgggagagactctgggagagaagtggaaagctcgtctgaatcagatgtcggccctggagttctactcttataaaaagtcaggtatcactgaagtgtgtagagaggaggctcgccgtgccctcaaggatggagtggccacaggaggacatgccgtatcccggggaagtgcaaagatcagatggttggaggagagaggatatctgcagccctatgggaaggttgttgacctcggatgtggcagagggggctggagctattatgccgccaccatccgcaaagtgcaggaggtgagaggatacacaaagggaggtcccggtcatgaagaacccatgctggtgcaaagctatgggtggaacatagttcgtctcaagagtggagtggacgtcttccacatggcggctgagccgtgtgacactctgctgtgtgacataggtgagtcatcatctagtcctgaagtggaagagacacgaacactcagagtgctctctatggtgggggactggcttgaaaaaagaccaggggccttctgtataaaggtgctgtgcccatacaccagcactatgatggaaaccatggagcgactgcaacgtaggcatgggggaggattagtcagagtgccattgtgtcgcaactccacacatgagatgtactgggtctctggggcaaagagcaacatcataaaaagtgtgtccaccacaagtcagctcctcctgggacgcatggatggccccaggaggccagtgaaatatgaggaggatgtgaacctcggctcgggtacacgagctgtggcaagctgtgctgaggctcctaacatgaaaatcatcggcaggcgcattgagagaatccgcaatgaacatgcagaaacatggtttcttgatgaaaaccacccatacaggacatgggcctaccatgggagctacgaagcccccacgcaaggatcagcgtcttccctcgtgaacggggttgttagactcctgtcaaagccttgggacgtggtgactggagttacaggaatagccatgactgacaccacaccatacggccaacaaagagtcttcaaagaaaaagtggacaccagggtgccagatccccaagaaggcactcgccaggtaatgaacatagtctcttcctggctgtggaaggagctggggaaacgcaagcggccacgcgtctgcaccaaagaagagtttatcaacaaggtgcgcagcaatgcagcactgggagcaatatttgaagaggaaaaagaatggaagacggctgtggaagctgtgaatgatccaaggttttgggccctagtggatagggagagagaacaccacctgagaggagagtgtcacagctgtgtgtacaacatgatgggaaaaagagaaaagaagcaaggagagttcgggaaagcaaaaggtagccgcgccatctggtacatgtggttgggagccagattcttggagtttgaagcccttggattcttgaacgaggaccattggatgggaagagaaaactcaggaggtggagtcgaagggttaggattgcaaagacttggatacattctagaagaaatgaatcgggcaccaggaggaaagatgtacgcagatgacactgctggctgggacacccgcattagtaagtttgatctggagaatgaagctctgattaccaaccaaatggaggaagggcacagaactctggcgttggccgtgattaaatacacataccaaaacaaagtggtgaaggttctcagaccagctgaaggaggaaaaacagttatggacatcatttcaagacaagaccagagagggagtggacaagttgtcacttatgctctcaacacattcaccaacttggtggtgcagcttatccggaacatggaagctgaggaagtgttagagatgcaagacttatggttgttgaggaagccagagaaagtgaccagatggttgcagagcaatggatgggatagactcaaacgaatggcggtcagtggagatgactgcgttgtgaagccaatcgatgataggtttgcacatgccctcaggttcttgaatgacatgggaaaagttaggaaagacacacaggagtggaaaccctcgactggatggagcaattgggaagaagtcccgttctgctcccaccacttcaacaagctgtacctcaaggatgggagatccattgtggtcccttgccgccaccaagatgaactgattggccgagctcgcgtctcaccaggggcaggatggagcatccgggagactgcctgtcttgcaaaatcatatgcgcagatgtggcagctcctttatttccacagaagagaccttcgactgatggctaatgccatttgctcggctgtgccagttgactgggtaccaactgggagaaccacctggtcaatccatggaaagggagaatggatgaccactgaggacatgctcatggtgtggaatagagtgtggattgaggagaacgaccatatggaggacaagactcctgtaacaaaatggacagacattccctatctaggaaaaagggaggacttatggtgtggatcccttatagggcacagaccccgcaccacttgggctgaaaacatcaaagacacagtcaacatggtgcgcaggatcataggtgatgaagaaaagtacatggactatctatccacccaagtccgctacttgggtgaggaagggtccacacccggagtgttg

SEQ ID NO: 6 - DNA, 22 bp, Artificial Sequence
  ProbeR1Zika
ggatgctccatcctgcccctgg

SEQ ID NO: 7 - DNA, 24 bp, Artificial Sequence
  ProbeR2Zika
ggagctgccacatctgcgcatatg

SEQ ID NO: 8 - DNA, 23 bp, Artificial Sequence
  ProbeR3Zika
gaacctgagggcatgtgcaaacc

SEQ ID NO: 9 - DNA, 20 bp, Artificial Sequence
  ProbeZikaUniversalPolyA
aaaaaagcaaacctatcatc

SEQ ID NO: 10 - DNA, 14 bp, Artificial Sequence
  ProbeZikaUniversal
gcaaacctatcatc

SEQ ID NO: 11 - DNA, 16 bp, Artificial Sequence
  ProbeZikaDegene1
cttcagctggtctgag

SEQ ID NO: 12 - DNA, 16 bp, Artificial Sequence
  ProbeZikaDegene2
tttcagctggtctaag

SEQ ID NO: 13 - DNA, 22 bp, Artificial Sequence
  Binding sequence for ProbeR1Zika
ccaggggcaggatggagcatcc

SEQ ID NO: 14 - DNA, 24 bp, Artificial Sequence
  Binding sequence for ProbeR2Zika
catatgcgcagatgtggcagctcc

SEQ ID NO: 15 - DNA, 23 bp, Artificial Sequence
  Binding sequence for ProbeR3Zika
ggtttgcacatgccctcaggttc

SEQ ID NO: 16 - DNA, 14 bp, Artificial Sequence
  Binding sequence for ProbeZikaUniversalPolyA and ProbeZikaUniversal
gatgataggtttgc

SEQ ID NO: 17 - DNA, 16 bp, Artificial Sequence
  Binding sequence for ProbeZikaDegene1 and ProbeZikaDegene2 (mismatch at positions 3 and 16)
ctcagaccagctgaag